(19) United States
(12) Patent Application Publication
(10) Pub. No.: US 2010/0144589 A1
(43) Pub. Date: Jun. 10, 2010

(54) METHODS OF PREDICTING CANCER LETHALITY USING REPLIKIN COUNTS

(76) Inventors: Samuel BOGOCH, New York, NY (US); Elenore S. BOGOCH, New York, NY (US); Samuel Winston BOGOCH, Oakland, CA (US); Anne Elenore BORSANYI, Brookline, MA (US)

Correspondence Address: KENYON & KENYON LLP, 1500 K STREET N.W., SUITE 700, WASHINGTON, DC, 20005, US

(21) Appl. No.: 12/538,027

(22) Filed: Aug. 7, 2009

Related U.S. Application Data
(63) Continuation-in-part of application No. 12/429,044, filed on Apr. 23, 2009.
Application No. 12/429,044 is a continuation-in-part of application No. PCT/US2009/041565, filed on Apr. 23, 2008.
(60) Provisional application No. 61/185,160, filed on Jun. 8, 2009.
(60) Provisional application No. 61/179,686, filed on May 19, 2009.
(60) Provisional application No. 61/172,115, filed on Apr. 23, 2009.
(60) Provisional application No. 61/143,618, filed on Jan. 9, 2009.
(60) Provisional application No. 61/087,354, filed on Aug. 8, 2008.

(51) Int. Cl. A61K 38/02; A61K 31/7088; C07K 2/00; C07K 16/18; C07H 21/04; C07H 21/02; A61P 35/00; A61P 35/04; C12Q 1/02; C12Q 1/68

(52) U.S. Cl. 514/2; 514/44 R; 530/300; 530/387.5; 530/389.7; 536/23.1; 536/24.5; 435/29; 435/6

(57) ABSTRACT

The present invention provides methods of predicting the relative growth rate, replication rate, or lethality of a first malignancy as compared to another malignancy or as compared to a plurality of malignancies, including methods of predicting the relative growth rate, replication rate, or relative lethality of a primary malignancy as compared to a metastatic malignancy, including comparing the concentration of Replikin sequences in the first malignancy with the concentration of Replikin sequences in another malignancy or malignancies and further provides Replikin peptides and Replikin Peak Genes identified within a malignancy for diagnostic, therapeutic, preventive, and predictive purposes.

This application claims priority to U.S. Provisional Appln. Ser. No. 61/185,160, filed Jun. 8, 2009, U.S. Provisional Appln. Ser. No. 61/179,686, filed May 19, 2009, U.S. Provisional Appln. Ser. No. 61/172,115, filed Apr. 23, 2009, U.S. application Ser. No. 12/429,044, filed Apr. 23, 2009, PCT/US2009/41565, filed Apr. 23, 2009, U.S. Provisional Appln. Ser. No. 61/143,618, filed Jan. 9, 2009, and U.S. Provisional Appln. Ser. No. 61/087,354, filed Aug. 8, 2008, each of which is incorporated herein by reference in its entirety. This application additionally incorporates herein by reference, each in its entirety: U.S. Provisional Appln. Ser. No. 61/054,010, filed May 16, 2008, U.S. application Ser. No. 12/108,458, filed Apr. 23, 2008, U.S. application Ser. No. 12/010,027, filed Jan. 18, 2008, U.S. Provisional Appln. Ser. No. 60/991,676, filed Nov. 30, 2007, U.S. application Ser. No. 11/923,559, filed Oct. 24, 2007, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, U.S. Provisional Appln. Ser. No. 60/880,966, filed Jan. 18, 2007, U.S. Provisional Appln. Ser. No. 60/853,744, filed Oct. 24, 2006, U.S. application Ser. No. 11/355,120, filed Feb. 16, 2006, U.S. application Ser. No. 11/116,203, filed Apr. 28, 2005, U.S. application Ser. No. 10/860,050, filed Jun. 4, 2004, now U.S. Pat. No. 7,442,761, U.S. application Ser. No. 10/189,437, filed Jul. 8, 2002, now U.S. Pat. No. 7,452,963, U.S. application Ser. No. 10/105,232, filed Mar. 26, 2002, now U.S. Pat. No. 7,189,800, U.S. application Ser. No. 09/984,057, filed Oct. 26, 2001, now U.S. Pat. No. 7,420,028, and U.S. application Ser. No. 09/984,056, filed Oct. 26, 2001, now U.S. Pat. No. 7,176,275.

### TECHNICAL FIELD OF THE INVENTION

This invention relates generally to identifying and quantifying the lethality of different histological types of malignancies and variations of lethality within these types. The invention is further directed to diagnosis, prevention and treatment of cancer.

### BACKGROUND OF THE INVENTION

Cancer is a class of diseases in which cells divide absent limits that normally control growth of cells in tissue. Uncontrolled cancer cell growth often leads to invasion and destruction of tissues adjacent to the cancer cells since cancer cells are typically capable of living in environments different from the tissue from which the cells were transformed. As a result, cancer cells often spread to other locations in the body where they may rapidly replicate causing additional tumors, resulting trauma, and sometimes death. The rate at which a line of cancer cells replicates is often a determining factor in the aggressiveness and eventual lethality of the cancer. Rates of replication for particular types of cancer are also considered in developing strategies for cancer therapy.

Nearly all cancer cells are abnormal in their genetic material as compared to cells from which they were transformed. Some progress has been made in developing therapies that more directly target the molecular abnormalities in cancer cells. These therapies ideally inhibit or kill cancer cells while not extensively damaging normal cells. Nevertheless, the progress that has been made in developing targeted therapies remains severely insufficient since about one-quarter of deaths in the United States in 2009 are expected to result from cancer.

Cancer prognosis has traditionally been based on histological identification of the type of cancer, as well as the stage of cancer development and the extent of progression of the disease in the body. More recently, histologic grading and the presence of specific molecular markers have become useful in prognosis and the development of individual treatments.

While histologic grading is useful for providing qualitative prognoses for patients suffering from a malignancy, histopathology does not provide a quantitative measure of the degree of malignancy or the rate of growth, rate of replication, aggressiveness, or lethality of a malignancy. Furthermore, histopathological procedures provide inconsistent measures of degrees of malignancies. Quantitative methods of determining the rate of growth, rate of replication, aggressiveness, or lethality of a malignancy are therefore needed.

Replikin peptides are a family of small peptides that have been correlated with the phenomenon of rapid replication in malignancies, as well as viruses, and other infectious organisms. The association of Replikin peptides with rapid replication has been described in U.S. Pat. No. 7,189,800, U.S. Pat. No. 7,176,275, U.S. application Ser. No. 11/355,120, and U.S. Pat. No. 7,442,761, among others. Both Replikin concentration (number of Replikins per 100 amino acids) and Replikin composition have been correlated with the functional phenomenon of rapid replication.

There continues to be a need in the art for methods of determining the source, aggressiveness and lethality of malignancies in order to design optimally appropriate therapies. Analysis of Replikin sequences in the genome of malignancies provides such methods. Additionally, a need for methods of preventing and treating aggressive malignancies continues to exist in the art. Replikin concentration in the genome of the cell or organism and Replikin sequences identified in malignancies provide methods of preventing and treating aggressive malignancies.

### SUMMARY OF THE INVENTION

In one aspect, the present invention provides methods of predicting the relative rate of growth, rate of replication, and/or lethality of a first malignancy as compared to a second malignancy or as compared to a plurality of other malignancies comprising comparing a Replikin Count (Replikin concentration) in a cell from the first malignancy with a Replikin Count (Replikin concentration) in a cell from the second malignancy, or with the Replikin Counts (Replikin concentrations) in a plurality of cells from a plurality of malignancies. In another aspect, the present invention further provides Replikin peptides and Replikin Peak Genes identified within the genome or within the expressed proteins of a cell from a first malignancy wherein said malignancy is predicted to have a higher rate of growth or replication or greater lethality than a second malignancy, or a higher rate of growth or replication or greater lethality than a plurality of other malignancies or than a mean, median, or average of a plurality of other malignancies.

A first non-limiting aspect of the invention provides a method of predicting the relative rate of growth of at least one first malignancy of a known or unknown type of malignancy as compared to at least one second malignancy of a known or unknown type of malignancy comprising: comparing a Replikin Count of the at least one first malignancy with a Replikin Count of at least one second malignancy; and predicting the at least one first malignancy to have a relative rate of growth that is faster than the relative rate of growth of the at least one second malignancy if the Replikin Count of the at least one first malignancy is greater than the Replikin Count of the at least one second malignancy. In a non-limiting embodiment of the first aspect of the invention, the at least one second malignancy is of an unknown type or of a different type of malignancy than the first malignancy. In another non-limiting embodiment, the first malignancy is a metastasis or metastases of the second malignancy.

In another non-limiting embodiment, the method of prediction further comprises determining a Replikin Count of the at least one first malignancy and a Replikin Count of the at least one second malignancy before the comparing step. In another non-limiting embodiment, the at least one first malignancy is a metastasis of the at least one second malignancy, is a malignancy of unknown type or unknown origin in a subject suffering from the at least one second malignancy, or is a malignant cell of the same malignancy as the at least one second malignancy, or the at least one first malignancy and the at least one second malignancy are metastases of a third malignancy.

In another non-limiting embodiment, the Replikin Count of the at least one first malignancy is the Replikin Count of a Replikin Peak Gene of the at least one first malignancy and the Replikin Count of the at least one second malignancy is a Replikin Count of a Replikin Peak Gene of the at least one second malignancy.

In another non-limiting embodiment, the Replikin Count of the at least one second malignancy is a mean Replikin Count of a plurality of malignancies or a mean Replikin Count of a plurality of cells of a malignancy, the Replikin Count of the at least one first malignancy is a mean Replikin Count of a plurality of malignancies or a mean Replikin Count of a plurality of cells of a malignancy, or the Replikin Count of the at least one second malignancy is a mean Replikin Count of a plurality of malignancies or a mean Replikin Count of a plurality of cells of a malignancy and the Replikin Count of the at least one first malignancy is a mean Replikin Count of a plurality of malignancies or a mean Replikin Count of a plurality of cells of a malignancy.

In a further non-limiting embodiment, the Replikin Count of the at least one second malignancy is a mean, median, or other average Replikin Count of a plurality of malignancies where the plurality of malignancies represents a survey of malignancies. In another non-limiting embodiment, the Replikin Count of the at least one first malignancy or the mean Replikin Count of the at least one first malignancy is greater than the mean Replikin Count of the at least one second malignancy plus the standard deviation of the mean Replikin Count of said at least one second malignancy.

In another non-limiting embodiment, the relative rate of growth correlates with relative lethality.

In another non-limiting embodiment, the Replikin Count of the at least one first malignancy is a Replikin Count of an expressed protein, protein fragment or peptide isolated from a cell of the at least one first malignancy or a Replikin Count of the genome, a gene, a gene fragment, or any other nucleic acid sequence isolated from a cell of the at least one first malignancy and the Replikin Count of the at least one second malignancy is a Replikin Count of an expressed protein, protein fragment or peptide isolated from a cell of the at least one second malignancy or a Replikin Count of the genome, a gene, a gene fragment, or any other nucleic acid sequence isolated from a cell of the at least one second malignancy.

In a further non-limiting embodiment, the Replikin Count of the at least one first malignancy is the highest Replikin Count among a plurality of Replikin Counts of a first plurality of malignancies of a first type and the Replikin Count of the at least one second malignancy is the highest Replikin Count among a plurality of Replikin Counts of a second plurality of malignancies of a second type. In a further non-limiting embodiment, a Replikin Count of the Replikin Peak Gene of the malignancy having the highest Replikin Count among a plurality of Replikin Counts of the first type of malignancy is further compared to the Replikin Count of the Replikin Peak Gene of the malignancy having the highest Replikin Count among a plurality of Replikin Counts of the second type of malignancy, and if the first type of malignancy has a higher Replikin Count of the Replikin Peak Gene than the second type of malignancy, then the first type of malignancy is predicted to have a greater lethality than the second type of malignancy.

In another non-limiting embodiment, the first malignancy or the second malignancy is a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, or a glioblastoma malignancy.

Another non-limiting embodiment of the first aspect of the invention provides a method of predicting the relative rate of growth of at least one first malignancy of a known or unknown type of malignancy as compared to at least one second malignancy of a known or unknown type of malignancy performed by a processor comprising: comparing a Replikin Count of the at least one first malignancy with a Replikin Count of at least one second malignancy; and predicting the at least one first malignancy to have a relative rate of growth that is faster than the relative rate of growth of the at least one second malignancy if the Replikin Count of the at least one first malignancy is greater than the Replikin Count of the at least one second malignancy. In a further non-limiting embodiment, at least one Replikin Count is outputted to a user or to a display. In another non-limiting embodiment, at least one representation of a prediction of the relative rate of growth of at least one first malignancy is outputted to a user or to a display.

Another non-limiting embodiment of the first aspect of the invention provides a machine-readable storage medium having stored thereon executable instructions that, when executed by a processor, cause the processor to provide sufficient data to a user, a display, or a printout, such that said user or a user of said display or printout may predict the relative rate of growth of at least one first malignancy according to an aspect of the invention for predicting the relative rate of growth of the at least one first malignancy.

Another non-limiting embodiment of the first aspect of the invention provides a computer system, including a processor coupled to a network and a memory coupled to the processor, the memory containing a plurality of instructions to perform a method of predicting the relative rate of growth of at least one first malignancy as compared to at least one second malignancy.

A second non-limiting aspect of the present invention provides a method of making a composition comprising at least one component of at least one first malignancy predicted to have a relative rate of growth faster than the relative rate of growth of at least one second malignancy, where the method comprises including the at least one component in the composition. In a non-limiting embodiment of the second aspect of the invention, the at least one component of the at least one first malignancy is at least one Replikin peptide or at least one Replikin Peak Gene identified within or isolated from the at least one first malignancy. In another non-limiting embodiment, the composition is an immunogenic composition. In another non-limiting embodiment, the immunogenic composition is a vaccine.

In another non-limiting embodiment of the second aspect of the present invention, the method of making a composition further comprises combining a pharmaceutically acceptable carrier or adjuvant or both with at least one component of at least one first malignancy predicted to have a relative rate of growth faster than the relative rate of growth of at least one second malignancy.

In a further non-limiting embodiment of the second aspect of the present invention, the at least one Replikin peptide or at least one Replikin Peak Gene are isolated from the first malignancy.

A third non-limiting aspect of the present invention provides a use of at least one component of a cell of at least one first malignancy predicted to have a relative rate of growth that is faster than the relative rate of growth of at least one second malignancy for the treatment of at least one first or second malignancy or for the treatment of a metastatic malignancy related to at least one first or second malignancy. In a non-limiting embodiment of the third aspect of the present invention, the at least one component of a cell of a first malignancy is at least one Replikin peptide or at least one Replikin Peak Gene identified in a cell of the first malignancy.

A non-limiting embodiment of the third aspect of the present invention provides a use of at least one component of a cell of at least one first malignancy predicted to have a relative rate of growth that is faster than the relative rate of growth of at least one second malignancy in the manufacture of a medicament for the treatment of malignancy. In a non-limiting embodiment, the at least one component is at least one Replikin peptide or at least one Replikin Peak Gene. In another non-limiting embodiment, the treatment of malignancy is treatment of at least one first malignancy or at least one second malignancy or a metastatic malignancy of at least one first malignancy or at least one second malignancy.

In a non-limiting embodiment of the third aspect of the present invention, at least one component of a cell is at least one Replikin peptide or at least one Replikin Peak Gene isolated from or identified in the cell.

A fourth non-limiting aspect of the present invention provides a method of treating a malignancy comprising administering to a subject at least one component of a cell of at least one first malignancy predicted to have a relative rate of growth that is faster than the relative rate of growth of at least one second malignancy. In a non-limiting embodiment of the fourth aspect of the present invention, the at least one component is at least one Replikin peptide or at least one Replikin Peak Gene identified in the at least one first malignancy. In a non-limiting embodiment, at least one component of a cell is at least one Replikin peptide or at least one Replikin Peak Gene isolated from or identified in the cell.

A fifth non-limiting aspect of the present invention provides a method of stimulating the immune system of a subject comprising administering to said subject, at least one component of at least one first malignancy predicted to have a relative rate of growth that is faster than the relative rate of growth of at least one second malignancy. In a non-limiting embodiment of the fifth aspect of the present invention, the at least one component is at least one Replikin peptide or at least one Replikin Peak Gene identified in the at least one first malignancy. In a non-limiting embodiment, at least one component of a cell is at least one Replikin peptide or at least one Replikin Peak Gene isolated from or identified in the cell.

A sixth non-limiting aspect of the present invention provides a method of making an antibody or an antibody fragment that binds to at least one antigenic component of at least one first malignancy, wherein said at least one first malignancy is predicted to have a relative rate of growth that is faster than the relative rate of growth of at least one second malignancy comprising identifying the antigenic component and making an antibody or antibody fragment that binds to the antigenic component. In a non-limiting embodiment of the sixth aspect of the present invention, the at least one antigenic component is at least one Replikin peptide or at least one Replikin Peak Gene identified in the first malignancy. In a non-limiting embodiment, the at least one antigenic component of a cell is at least one Replikin peptide or at least one Replikin Peak Gene isolated from or identified in the cell.

A seventh non-limiting aspect of the present invention provides a method of making at least one siRNA comprising: identifying at least one Replikin peptide or at least one Replikin Peak Gene in at least one first malignancy predicted to have a relative rate of growth that is faster than the relative rate of growth of at least one second malignancy; and making said at least one siRNA to be complementary to at least a portion of a nucleic acid that encodes said at least one Replikin peptide or said at least one Replikin Peak Gene.

An eighth non-limiting aspect of the present invention provides a method of determining the relative rate of increase in the relative rate of growth of at least one first malignancy as compared to the relative rate of increase in the relative rate of growth of at least one second malignancy comprising: comparing the standard deviation of the mean Replikin Count of a plurality of cells of said at least one first malignancy to the standard deviation of the mean Replikin Count of a plurality of cells of said at least one second malignancy; and predicting that the relative increase in the relative rate of growth of said at least one first malignancy is greater than the relative rate of increase in the relative rate of growth of said at least one second malignancy if the standard deviation of the mean Replikin Count of the plurality of cells of the at least one first malignancy is greater than the standard deviation of the mean Replikin Count of a plurality of cells of the at least one second malignancy.

In a non-limiting embodiment of the eighth aspect of the present invention, the at least one first malignancy may be a metastasis of the at least one second malignancy or the at least one first malignancy, or may be a malignancy of unknown origin or unknown type in a subject suffering from the at least one second malignancy. In another non-limiting embodiment, the at least one first malignancy and the at least one second malignancy are both metastases of a third malignancy. In another non-limiting embodiment, the at least one first malignancy is a plurality of malignancies differing from the at least one second malignancy, or the at least one second malignancy is a plurality of malignancies differing from the at least one first malignancy, or the at least one first malignancy and the at least one second malignancy are both a plurality of malignancies differing one from the other. In another non-limiting embodiment, the at least one first malignancy is a plurality of malignancies of a first type of malignancy and the at least one second malignancy is a plurality of malignancies of a second type of malignancy.

A ninth non-limiting aspect of the present invention provides a method of predicting an expansion of a malignancy comprising: determining a mean Replikin Count and a standard deviation of said mean Replikin Count for a plurality of cells of a first malignancy for a first time period in a first anatomic region or for a first plurality of malignancies; determining a Replikin Count of at least one other cell, said at least one other cell being distinct from the plurality of cells and being at least one cell of the first malignancy, of a metastasis of the first malignancy, of a malignancy of unknown origin or unknown type in a subject suffering from the first malignancy at a second time period and/or in a second anatomic region, wherein said second time period is different from said first time period and/or said second anatomic region is different from said first anatomic region, or of a second malignancy in a different subject; and predicting an expansion of said first malignancy, said metastasis, said malignancy of unknown origin or unknown type, or said second malignancy in a different subject if the Replikin Count of the at least one other cell is greater than the mean Replikin Count of the plurality of cells plus one standard deviation of the mean Replikin Count of the plurality of cells, or if the Replikin Count of said at least one other cell is greater than the mean Replikin Count of the first plurality of malignancies plus one standard deviation of the mean.

In a non-limiting embodiment of the ninth aspect of the present invention, the at least one other cell is a second plurality of cells from the second time period and/or the second anatomic region or said second malignancy in a different subject, and the Replikin Count of each cell of the plurality of cells from the second time period and/or second anatomic region or said second malignancy in a different subject is compared separately to the mean Replikin Count plus one standard deviation of said mean Replikin Count. In a further non-limiting embodiment, the expansion of the first malignancy in the second time period and/or the second anatomic region or the expansion of said second malignancy in a different subject is predicted if the number of Replikin Counts of the second plurality of cells that is greater than the mean Replikin Count of the first plurality of cells plus one standard deviation of the mean of the Replikin Count is greater than the number of Replikin Counts of the second plurality of cells that is less than the mean Replikin Count of the first plurality of cells or the first plurality of malignancies minus one standard deviation of the mean.

A tenth non-limiting aspect of the present invention contemplates a kit for providing a prognosis of a patient suffering from a malignancy, said prognosis concerning the lethality of the malignancy, comprising: a formula for determining the lethality of a malignancy based on a Replikin Count in at least one cell of the malignancy. In a non-limiting embodiment of the tenth aspect of the invention, the formula in the kit is derived from a survey of a plurality of malignancies of a first type of malignancy. In another non-limiting embodiment, the malignancy is a glioblastoma malignancy, a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, or a non-small cell lung carcinoma malignancy. In a non-limiting embodiment, the kit comprises software containing the formula.

An eleventh non-limiting aspect of the present invention provides a method of predicting the lethality of a first malignancy comprising: determining a Replikin Count of a first malignancy performing a regression analysis with the Replikin Count of a plurality of second malignancies versus the lethality of the plurality of second malignancies; and predicting the lethality of the first malignancy based on the regression analysis of the plurality of second malignancies. In a non-limiting embodiment of the eleventh aspect of the invention, the regression analysis of the plurality of second malignancies is performed using the Replikin Count of each individual second malignancy of the plurality of second malignancies and a patient survival outcome for each individual second malignancy. In another non-limiting embodiment, the patient survival outcome is a length of survival of patient following diagnosis of the second malignancy. In another non-limiting embodiment, the method is performed by a processor wherein at least one representation of the prediction of lethality is outputted to a user or display.

Another non-limiting embodiment of the eleventh aspect of the invention provides a machine-readable storage medium having stored thereon executable instructions that, when executed by a processor, cause the processor to provide sufficient data to a user, a display, or a printout such that said user or a user of said display or said printout may predict the lethality of a malignancy based on the regression analysis. Another non-limiting embodiment provides a computer system, comprising: a processor coupled to a network; a memory coupled to the processor, the memory containing a plurality of instructions to perform the method of predicting the lethality of a malignancy based on the regression analysis.

### BRIEF DESCRIPTION OF THE DRAWINGS

FIG. 1 illustrates a quantitative relationship between the concentration of Replikin peptides in the Replikin Peak Gene of individual proteins associated with cancer cells of a plurality of common human malignancies and five-year mortality rates for each of the plurality of common human malignancies. Replikin Count was determined from the highest Replikin Count identified in a Replikin Peak Gene of sequences surveyed at www.pubmed.com. The five-year mortality rates are as reported in Brenner, H., “Long-term survival rates of cancer patients achieved by the end of the 20th century: a period analysis,” The Lancet, 360 (Oct. 12, 2002), 1131-1135. The lowest Replikin concentrations are seen in thyroid cancer (15 Replikin sequences per 100 amino acids) and in prostate cancer (20 Replikin sequences per 100 amino acids) and the lowest five-year mortality rates are seen in thyroid cancer (2%) and prostate cancer (3%). The highest Replikin concentrations are seen in non-small cell lung carcinoma (250 Replikin sequences per 100 amino acids) and in glioblastoma (324 Replikin sequences per 100 amino acids) and the highest five-year mortality rates are seen in non-small cell lung carcinoma (92%) and glioblastoma (99%). These data illustrate a relationship between Replikin concentration in a given type of cancer and lethality in that type of cancer as compared to the Replikin concentration and lethality in other types of cancer.

FIG. 2 illustrates polynomial regression analysis of the data presented in FIG. 1 and in Table 1. The regression formula provided by the regression analysis is y=0.0401x²=1.3041x+35.812 with an r² value of 0.9089.

FIG. 3 illustrates a direct sequential correlation between Replikin concentration of isolates of taura syndrome virus (TSV) collected from Belize, Thailand, Hawaii and Venezuela, respectively, and mean number of days to 50% mortality in Litopenaeus vannamei shrimp challenged with the respective TSV isolates. Statistical differences between the Replikin concentration for each isolate are significant at a level of p<0.001. The data illustrated in FIG. 3 are described in Example 7 below.

FIG. 4 illustrates a direct correlation between Replikin concentration in isolates of taura syndrome virus (TSV) collected from Belize, Thailand, Hawaii and Venezuela, respectively, and mean cumulative survival of Litopenaeus vannamei shrimp at 15 days after challenge with respective TSV isolates. Statistical differences between the Replikin concentrations for each isolate are significant at a level of p<0.001. The data illustrated in FIG. 4 are described in Example 7 below.

### DETAILED DESCRIPTION OF THE INVENTION

### Definitions

As used herein, a “Replikin Peak Gene (RPG)” means a segment of a genome, protein, segment of protein, or protein fragment in which an expressed gene or gene segment has a highest concentration of continuous, non-interrupted and/or overlapping Replikin sequences (number of Replikin sequences per 100 amino acids) when compared to other segments or named genes of the genome. Generally, a whole protein or gene or gene segment that contains the amino acid portion (or expressed amino acid portion) having the highest concentration of continuous Replikin sequences is also referred to as the Replikin Peak Gene. More than one RPG may be identified within a gene, gene segment, protein, or protein fragment. An RPG may have a terminal lysine or a terminal histidine, two terminal lysines, or a terminal lysine and a terminal histidine. For diagnostic, therapeutic and preventive purposes, an RPG may have a terminal lysine or a terminal histidine, two terminal lysines, or a terminal lysine and a terminal histidine, or may have neither a terminal lysine nor a terminal histidine so long as the terminal portion of the RPG contains a Replikin sequence or Replikin sequences defined by the definition of a Replikin sequence, namely, an amino acid sequence having about 7 to about 50 amino acids comprising:
(1) at least one lysine residue located six to ten amino acid residues from a second lysine residue;
(2) at least one histidine residue; and
(3) at least 6% lysine residues.
   Further, for diagnostic, therapeutic, preventive and predictive purposes, an RPG may include the protein or protein fragment that contains an identified RPG. For predictive purposes, a Replikin Count in the RPG may be used to identify relative rates of replication and/or lethality. Likewise, the RPG may be used as an immunogenic compound or as a vaccine. Whole proteins or protein fragments containing RPGs are likewise useful for diagnostic, therapeutic and preventive purposes, such as, for example, to be included in immunogenic compounds, vaccines and for production of therapeutic or diagnostic antibodies.

As used herein, a “Replikin sequence” is an amino acid sequence of 7 to about 50 amino acids comprising or consisting of a Replikin motif wherein the Replikin motif comprises:
(1) at least one lysine residue located at a first terminus of said isolated peptide and at least one lysine residue or at least one histidine residue located at a second terminus of said isolated peptide;
(2) a first lysine residue located six to ten residues from a second lysine residue;
(3) at least one histidine residue; and
(4) at least 6% lysine residues.
   For the purpose of determining Replikin concentration, a Replikin sequence must have a lysine residue at one terminus and a lysine or a histidine residue at the other terminus. For diagnostic, therapeutic, and preventive purposes, a Replikin sequence may or may not have defined termini.

The term “Replikin sequence” can also refer to a nucleic acid sequence encoding an amino acid sequence having about 7 to about 50 amino acids comprising:
(1) at least one lysine residue located six to ten amino acid residues from a second lysine residue;
(2) at least one histidine residue; and
(3) at least 6% lysine residues,
   wherein the amino acid sequence may comprise a terminal lysine and may further comprise a terminal lysine or a terminal histidine.

As used herein, the term “peptide” or “protein” refers to a compound of two or more amino acids in which the carboxyl group of one amino acid is attached to an amino group of another amino acid via a peptide bond. As used herein, “isolated” or “synthesized” peptide or protein or biologically active portion of a peptide or protein refers to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silico from nucleic acid or amino acid sequences that are available through public or private databases or sequence collections. An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well-known in the art that, due to redundancy in the genetic code, individual nucleotides can be readily exchanged in a codon and still result in an identical amino acid sequence. As will be understood by one of skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

As used herein, “Replikin Count” or “Replikin Concentration” refers to the number of Replikins per 100 amino acids in a protein, protein fragment, or genome of a cell or virus.

As used herein, the term “continuous Replikin sequences” means a series of two or more Replikin sequences that are overlapped or are directly covalently linked or are both overlapped and directly covalently linked.

As used herein, the term cancer “type” refers to malignancies that share histology or origin. One of ordinary skill in the art knows how to separate different malignancies by cancer “type.” Malignancies subject to aspects of the invention may be of the same cancer type or of different cancer types. The malignancies may also be of unknown type or may be metastatic and of known or unknown type. Many cancers histologically diagnosed in a primary malignancy are of unknown cancer type such as when a metastasis that is being examined has changed and has become difficult or impossible to type by histological methods. In such cases, the present methods of prediction are of use as an independent method of predicting the relative rate of replication and lethality of unknown and metastatic cancers. The methods of prediction of relative replication rate and/or lethality disclosed herein provide a tool for predicting the relative rate of growth, relative replication rate, and/or relative lethality of malignancies that are of unknown type or of unknown histological origin and/or are metastatic using the Replikin Count of any malignancy whether of known or unknown cancer type.

As used herein, “homologous” or “homology” or “sequence identity” are used to indicate that a nucleic acid sequence or amino acid sequence exhibits substantial structural or functional equivalence with another sequence. Any structural or functional differences between sequences having sequence identity or homology will be de minimus; that is, they will not affect the ability of the sequence to function as indicated in the desired application. Differences may be due to inherent variations in codon usage among different species, for example. Structural differences are considered de minimus if there is a significant amount of sequence overlap or similarity between two or more different sequences or if the different sequences exhibit similar physical characteristics even if the sequences differ in length or structure. Such characteristics include, for example, the ability to hybridize under defined conditions, or in the case of proteins, immunological crossreactivity, similar enzymatic activity, etc. The skilled practitioner can readily determine each of these characteristics by art known methods.

To determine the percent identity or percent homology of two sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

The nucleic acid and protein sequences of the present invention can further be used as a “query sequence” to perform a search against sequence databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program. BLAST protein searches can be performed with the XBLAST program to obtain amino acid sequences homologous to the proteins of the invention. To obtain gapped alignments for comparison purposes, Gapped BLAST can be utilized as described in Altschul et al. (1997) Nucleic Acids Res. 25(17):3389-3402. When utilizing BLAST and gapped BLAST programs, the default parameters of the respective programs (e.g., XBLAST and NBLAST) can be used.

As used herein a “vaccine” is any substance, compound, composition, mixture, or other therapeutic substance that, when administered to a human or animal via any method of administration known to the skilled artisan now or hereafter, produces an immune response, a humoral response, an antibody response, or a protective effect in the human or animal.

As used herein, the terms, “growth,” “replication,” “aggressiveness,” and “lethality” are interchangeable for the purposes of providing a prediction of the behavior of a malignancy. The rate of growth, rate of replication, aggressiveness, or lethality of a malignancy, for the purposes of providing predictions based on Replikin concentrations, may be considered to correlate.

### Relative Rate of Growth, Rate of Replication, and Lethality Among Cancer Cells

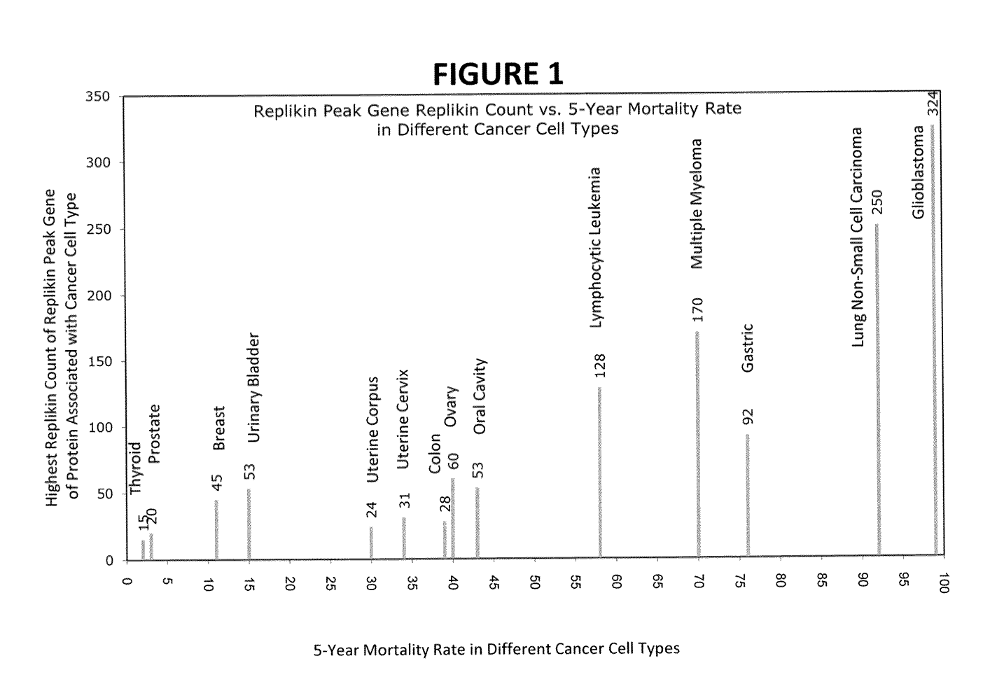

An embodiment of predictive aspects of the present invention provides methods of predicting the relative rate of growth, the relative rate of replication, and/or the relative lethality of a first malignancy as compared to a second malignancy or as compared to a plurality of malignancies comprising: comparing a Replikin Count in the first malignancy with a Replikin Count in the second malignancy or a mean, median, mode (or other average), range, or other aggregating or segregating measure of Replikin Count in a plurality of malignancies. The present invention further provides methods of predicting relative rate of growth, relative rate or replication, and/or relative lethality in a primary malignancy or metastatic malignancy, whether such primary malignancy or metastatic malignancy is of known or unknown type or origin. The Replikin Count is a measure of the Replikin concentration of a malignancy and is the number of Replikin sequences identified in the genome of the malignancy or in a segment of the genome of the malignancy or identified in a protein or protein fragment of the malignancy per 100 encoded or expressed amino acids. A malignancy having a higher Replikin concentration has been demonstrated to have a relatively higher rate of replication or growth and relatively greater lethality. See FIG. 1. Likewise, a malignancy having a lower Replikin concentration has been demonstrated to have a relatively lower rate of replication or growth and relatively lower lethality. See id.

An embodiment of the present invention also provides Replikin peptides or Replikin Peak Genes identified within the genome or within the expressed proteins of a cell from at least one first malignancy wherein said malignancy is predicted to have a higher rate of growth or replication or greater lethality than a second malignancy or a higher rate of growth or replication or greater lethality than a plurality of other malignancies.

The relative rate of growth, replication, or lethality of a malignancy may be predicted by comparing the Replikin Count of a first malignancy to the Replikin Count of a second malignancy. A cell from a first malignancy may have a higher or lower Replikin Count than a cell from a second malignancy, a higher or lower Replikin Count than a mean Replikin Count of a plurality of cells from a single malignancy, a plurality of malignancies, a plurality of cells from a single patient suffering from a malignancy (whether the malignant cells are of known or unknown histology or origin), each of the plurality of cells from a malignancy or a plurality of other malignancies, or a plurality of cells from the plurality of other malignancies. Higher Replikin Counts represent a higher rate of growth, replication, and/or greater lethality. Lower Replikin Counts represent a lower rate of growth, replication, and/or lower lethality.

A first malignancy may be predicted to have a higher rate of growth, replication, and/or a greater lethality as compared to a plurality of other malignancies if a cell from the first malignancy has a higher Replikin Count than cells from the plurality of the other malignancies. A first malignancy may likewise be predicted to have higher growth, replication, and/or lethality if a cell from the first malignancy has a higher Replikin Count than the mean of the Replikin Counts of cells from the plurality of the other malignancies or than a plurality of means of a plurality of the other malignancies.

A first malignancy may be predicted to have a lower rate of growth, replication, and/or a lower lethality as compared to a plurality of other malignancies if a cell from the first malignancy has a lower Replikin Count than cells from the plurality of the other malignancies. A first malignancy may likewise be predicted to have lower growth, replication, and/or lethality if a cell from the first malignancy has a lower Replikin Count than the mean of the Replikin Counts of cells from the plurality of the other malignancies or than a plurality of means of a plurality of the other malignancies.

In an embodiment of a predictive method of the invention, the relative rate of growth, relative rate of replication, or relative lethality of a malignancy may be determined by comparing a Replikin Count of at least one cell of a first malignancy to a Replikin Count of at least one other cell of the first malignancy. The method may apply to a metastatic cell of a first malignancy where the one other cell is a different metastatic cell of the first malignancy, is a non-metastatic cell of the first malignancy, or is a malignant cell of unknown origin within a patient suffering from the first malignancy.

The Replikin Count of a first malignancy may be compared to a plurality of malignancies or to a mean or average of the Replikin Count of a plurality of malignancies. The Replikin Count of a first malignancy may also be compared to a plurality of means and/or averages of the Replikin Count of a plurality of malignancies.

A mean, median, mode (or other average), range, or other aggregating measure of Replikin Count may be used when analyzing a plurality of Replikin Counts. Likewise, any other tool known to one of skill in the art now and hereafter may be used to aggregate or segregate the Replikin Counts of particular malignancies or particular cells within a malignancy.

The rate of growth, rate of replication, and/or lethality of a malignancy or of a type of malignancy may be determined by any method known to one of skill in the art now or hereafter. A rate of growth may be determined by, for example, the rate of increase in volume of a tumor (or any other measure of size including diameter, circumference, etc.), the rate of increase in mass of a tumor, the rate of spread of a tumor, the rate of metastasis of tumor, etc. A rate of replication may be determined by, for example, any measure of the rate of growth as well as any measure of the rate of increase in number of cells from a cell, or tumor, or mass of a malignancy. A rate of lethality may be determined, for example, by any measure of growth, or replication, or any measure of metastasis of a malignancy, life expectancy with a malignancy, invasiveness of a malignancy, or rate of death of a subject suffering from a malignancy. One measure of lethality, among many other possible measures, is the five year mortality rate.

### Computer Methods for Determining Relative Rate of Growth, Rate of Replication, and Lethality Among Cancer Cells

A prediction of the relative rate of growth, replication, and/or lethality of a malignancy may be performed by a processor. A prediction may be output to a user or display. Likewise, a particular Replikin peptide or Replikin Peak Gene within a malignancy predicted to have a higher rate of growth, replication or lethality may be output to a user or display. A machine-readable storage medium may contain executable instructions that, when executed by a processor, cause the processor to provide sufficient data to a user, a printout, or a display such that the user or a user of the printout or display may predict the relative rate of growth, replication, or lethality of a malignancy. A process for predicting a relative rate of growth may comprise: comparing a Replikin Count of at least one first malignancy with a Replikin Count of at least one second malignancy; and predicting the first malignancy to have a relative rate of growth that is faster than the relative rate of growth of the second malignancy if the Replikin Count of the first malignancy is greater than the Replikin Count of the second malignancy.

A computer system may include a processor coupled to a network, and a memory coupled to a processor, wherein the memory contains a plurality of instruction to perform the methods of prediction discussed herein.

A user of outputted data from a processor, storage medium, machine-readable medium, or computer system may include any person or any machine that records or analyzes the outputted data. A display or printout may include any mechanism by which data is outputted so that any person or any machine may record or analyze the outputted data, including a printed document, a visual impulse, an aural impulse, or any other perceivable impulse, a computer monitor, a set of numbers, or any other display or printout of data including a digital recording medium.

Outputs of data may include an output of a prediction of the relative growth rate, replication rate or lethality of a malignancy, a plurality of malignancies, or a cell or plurality of cells of a malignancy. Outputs of data may also include a Replikin Count or a number or numbers sufficient for a user to determine a Replikin Count, any portion or component of a cell of a malignancy or of a malignancy identified by a processor as predicted to have a higher or lower rate of growth or replication or a higher or lower lethality, including a Replikin peptide or a Replikin Peak Gene of a malignancy, or any information that will assist a user in providing a prediction concerning the relative rate of growth, replication, and/or lethality of a malignancy or that will assist a user in providing any portion or component of a cell or a malignancy identified by a processor as predicted to have a higher or lower rate of growth or replication or a higher or lower lethality.

A representation of a prediction of the relative rate of growth of a malignancy is outputted to a user or to a display when any information is outputted to a user or display that provides sufficient information for the user or a machine receiving the output or display to determine or provide a prediction concerning the relative rate of growth, replication, or lethality of a malignancy, a plurality of malignancies, a cell of a malignancy, or a plurality of cells of a malignancy. Sufficient information to provide a prediction includes but is not limited to a Replikin Count, a prediction, a number or symbol or image that signifies a prediction, a listing of a cell, malignancy or symbol of a cell or malignancy that is predicted to have a lower or higher rate of growth, replication or lethality, or any other piece of information or collection of information to provide a user or display sufficient information to determine or provide a prediction concerning the relative rate of growth, replication, or lethality of a malignancy, a plurality of malignancies, a cell of a malignancy, or a plurality of cells of a malignancy.

Predictions May be Made by Comparing Cells from Cancer Types that are the Same or Different in Histology or Origin, Including Unknown Cancer Types

Malignancies subject to aspects of the invention may be of the same cancer type or of different cancer types where cancer types may be determined by histology, tissue origin, genetic makeup or anomaly, biochemical makeup or structure, metastatic origin from a primary, secondary, tertiary (or other) malignancy, by time or progression of a malignancy, by anatomic position of a malignancy, or by any other method known to one of skill in the art now or hereafter whereby the skilled artisan may differentiate cancer type. Malignancies may also be of unknown type or origin or may be metastatic and of known or unknown type or origin.

The methods of prediction of relative growth, replication, and/or lethality disclosed herein provide a tool for predicting the relative rate of growth, relative rate of replication, and/or relative lethality of malignancies that are of unknown type or of unknown histological origin and/or are metastatic using the Replikin Count of any malignancy whether of known or unknown cancer type. Many cancers histologically diagnosed in a primary malignancy are of unknown cancer type such as when a metastasis that is being examined has changed and has become difficult or impossible to type by histological methods. In such cases, the present methods of prediction are of use as an independent method of predicting the relative rate of replication and lethality of unknown and metastatic cancers.

An embodiment of the present invention provides a method of predicting the relative rate of growth of at least one first malignancy of an unknown or known type of malignancy comprising: comparing a Replikin Count of the at least one first malignancy with a Replikin Count of at least one second malignancy of an unknown type or of a different type of malignancy than the at least one first malignancy; and predicting the at least one first malignancy has a relative rate of growth that is faster than the relative rate of growth of the at least one second malignancy if the Replikin Count of the at least one first malignancy is greater than the Replikin Count of the at least one second malignancy. A further embodiment of the present invention provides for a determination of the Replikin Count of the at least one first malignancy and a Replikin Count of the at least one second malignancy. The Replikin Count of a malignancy may be determined in any portion of the genome or in any expressed protein or protein fragment of the malignancy. Comparison of the Replikin Count of two malignancies may be made between whole genomes of the malignancies, genome fragments of the malignancies, or expressed proteins or protein fragments of the malignancies. When Replikin Counts of individual cells or mean Replikin Counts of pluralities of cells are compared, comparable fragments of the genome or comparable expressed proteins or expressed protein fragments of the cells may be compared. For example, the Replikin Count of a particular expressed protein or of a Replikin Peak Gene of a particular expressed protein or protein fragment in the individual cells may be compared.

The relative growth or replication rate or relative lethality of a malignancy may be determined in any malignant cell, malignant tissue or tumor, or any malignancy in a patient (human or animal) suffering from a malignancy including a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, a glioblastoma malignancy, or any other kind of malignancy.

Any Replikin Count in individual cells may be compared. Over a mean Replikin Count, Replikin Counts of different sections of the genome or different expressed proteins or protein fragments of individual cells may be compared. With respect to mean Replikin Counts determined from a plurality of cells, any available information providing sequences of the genome or sequences of proteins or expressed protein fragments of a malignancy or malignancies may be compared. Such information may include sequencing of the genome of a malignancy, cDNA sequencing of a malignancy, sequencing of mRNAs of a malignancy or any other nucleic acid sequence of a malignancy. Such information may also include sequencing of expressed proteins, protein fragments, or peptides of a malignancy. Such information may further include in silico information providing nucleic acid or amino acid sequences of a malignancy or malignancies including research data, published journals, databases that contain sequence information such as those found at www.pubmed.com, or any other source of sequence information.

In comparing Replikin Counts between individual cells of a malignancy or cells from different malignancies or cells from a primary malignancy and metastatic cells from the same malignancy or unknown cancer cells in a patient suffering from a primary malignancy, a Replikin Count may be a mean Replikin Count of a plurality of cells of a malignancy, a mean Replikin Count of a plurality of cells of different malignancies, a mean Replikin Count of a plurality of cells from a primary malignancy and metastases of the malignancy, or unknown cancer cells from a patient suffering from a malignancy. A Replikin Count may also represent only a Replikin Count from a single cell in any one of the malignancies discussed above and herein. One of ordinary skill in the art would understand how to compare the Replikin Count of individual cells to the mean Replikin Count of a plurality of cells or mean Replikin Counts between different groupings of pluralities of cells. In an embodiment of a predictive method of the invention, a mean Replikin Count of a plurality of malignancies or a plurality of cells of a malignancy may be compared to a Replikin Count of single cell of a malignancy or a mean Replikin Count of a plurality of cells of various malignancies (including, for example, metastases of a primary malignancy) or the same malignancy may be compared to a mean Replikin Count of a plurality of cells of various malignancies (including, for example, metastases of a primary malignancy) or the same malignancy.

A plurality of malignancies may represent a survey of malignancies of a particular type of cancer. A plurality may also represent a survey of malignancies of unknown types of cancer. A plurality may also represent a survey of malignancies of all types of cancer. Such surveys provide baseline data for Replikin Counts and standard deviations from Replikin Counts among malignancies of known, unknown, and all types of cancer. The Replikin Counts with standard deviations may then be compared to a Replikin Count from a specific malignancy. If the Replikin Count of a specific malignancy is greater or less than that of a survey of cancers of the same type, the specific malignancy may be predicted to have a higher or lower rate of growth, rate of replication, or lethality, respectively, than the mean, median, or other average rates of growth, replication or lethality of the type of cancer. Likewise if the Replikin Count of a specific malignancy is greater or less than that of a survey of cancers of unknown type, and the specific malignancy is of known or unknown type, the specific malignancy may be predicted to have a higher or lower rate of growth, rate of replication, or lethality, respectively, than the mean, average, or median rates of growth, replication or lethality of the unknown types of cancer. Further, if the Replikin Count of a specific malignancy is greater or less than that of a survey of cancers of all types, the specific malignancy may be predicted to have a higher or lower rate of growth, rate of replication, or lethality, respectively, than the mean, median, or other average rates of growth, replication or lethality of all types of cancer included in the survey.

A survey may represent data collected from any number of malignancies from one to as many malignancies as may be included in the data set of survey. A survey may collect data on Replikin Count as determined in any portion of the genome of cells of a malignancy or any portion of the expressed proteins and protein fragments of the cells of a malignancy. A survey may collect data on rate of growth or replication of a malignancy, rate of expansion of volume or other measure of size of a tumor of a malignancy, rate of increase in mass of a tumor of a malignancy, lethality of a malignancy including time to death or percent mortality at a specific time of a malignancy. A survey may likewise collect any data understood now or hereafter by one of skill in the art to reflect on Replikin sequences present in a malignancy and/or rates of growth, replication, and/or lethality of a malignancy.

One aspect of a survey provides baseline data against which a Replikin Count of an individual malignancy or a plurality of malignancies may be compared to predict the rate of growth, replication, or lethality of a malignancy or group of malignancies.

The relative rate of growth of a cell or a tissue in which a Replikin Count (or a plurality of Replikin Counts) has been determined may be compared to the relative rate of replication of the cell or tissue. As is understood by one of ordinary skill in the art, the relative rate of growth and the relative rate of replication of a malignancy may also be correlated with the relative lethality of a malignancy. As such, an embodiment of a method of prediction of the present invention provides methods of predicting or determining the relative rate of growth of a malignancy, the relative rate of replication of a malignancy, or the relative lethality of a malignancy.

In a further embodiment, the first malignancy may be a primary malignancy and the relative rate of growth, the relative rate of replication, and/or the relative lethality of the primary malignancy is compared to any said second malignancy. In a further embodiment, the first malignancy may be a primary malignancy and the second malignancy may be a metastatic malignancy. In a further embodiment, the primary malignancy and the metastatic malignancy (or two metastatic malignancies) may be compared to yet another malignancy or plurality of malignancies.

A method of predicting the relative replication rate or relative lethality of a malignancy may predict the relative replication rate or relative lethality of any malignancy or group of malignancies of a certain cancer type or any malignancy or group of malignancies of an unknown type so long as the individuals of the group of malignancies share similar, or otherwise aggregatable or segregatable Replikin concentrations in their genome, in a segment of their genome, in a protein or proteins, or in a fragment or fragments of a protein or a plurality of their proteins. A method of predicting may be applied to a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, a glioblastoma malignancy, or any human or animal malignancy wherein the Replikin Count of the malignancy is known or may be known or may be established by methods herein described.

### Replikin Counts in Replikin Peak Genes Predict Relative Rates of Growth and Relative Lethality

Replikin concentration in a malignancy may be determined in the Replikin Peak Gene of a malignancy. The Replikin Peak Gene of a malignancy is the segment of a genome, protein, or protein fragment of a malignancy that has a highest concentration of continuous, non-interrupted and/or overlapping Replikin sequences as compared to other segments of the genome of the malignancy or as compared to expressed proteins or protein fragments of the malignancy.

When comparing the Replikin Count of a first malignancy to the Replikin Count of a second malignancy, the Replikin Count of a Replikin Peak Gene of the first malignancy may be compared to the Replikin Count of a Replikin Peak Gene of the second malignancy. The Replikin Peak Gene may be a genome segment, a protein, or a protein fragment. The Replikin Count of a first malignancy may also be compared to a plurality of malignancies or to a mean or average of the Replikin Count of a plurality of malignancies. The Replikin Count of a first malignancy may also be compared to a plurality of means or other averages or methods of aggregation or segregation of the Replikin Count of a plurality of malignancies.

A prediction of the relative rate of growth, replication, or lethality of a malignancy may be performed by a processor. A prediction may be output to a user or display. Likewise, a particular Replikin peptide or Replikin Peak Gene within a malignancy that is predicted to have a higher rate of growth, replication or lethality may be output to a user or display.

An embodiment of a predictive method of the present invention provides a method of predicting the relative rate of growth, relative rate of replication, or the relative lethality of a first malignancy as compared to a second malignancy or as compared to a plurality of other malignancies comprising (1) identifying some or all Replikin sequences in the genome, in a protein, or in a protein fragment of a first malignancy, (2) identifying some or all Replikin sequences in the genome, in a protein, or in a protein fragment of the second malignancy or in the genome, in a protein, or in a protein fragment of each of a plurality of other malignancies, (3) determining a Replikin Count of the segment of the genome, of the protein, or of the protein fragment of the first malignancy that has the highest concentration of continuous non-interrupted and/or overlapping Replikin sequences when compared to other segments of the genome, proteins, or protein fragments of the first malignancy, (4) determining a Replikin Count of the segment of the genome, of the protein, or of the protein fragment of the second malignancy or of each of the plurality of other malignancies that has or have the highest concentration of continuous non-interrupted and/or overlapping Replikin sequences when compared to other segments of the genome, proteins, or protein fragments of the second malignancy or of each of the plurality of other malignancies, (5) identifying the first malignancy as having a higher Replikin Count than the second malignancy or than each of the plurality of other malignancies, and (6) predicting the first malignancy to have a relatively higher rate of replication or a relatively greater lethality than the second malignancy or than the plurality of other malignancies.

### Methods of Comparing Relative Lethality of Cancer Cells, Tissues, or Types

An embodiment of predictive methods of the present invention provides a method of predicting the relative replication and/or relative lethality of a first type of malignancy comprising (1) analyzing publicly available nucleotide or amino acid sequences of at least a first type and a second type of malignancy published at www.pubmed.com, (2) determining the Replikin Count for each accession number with a nucleotide or amino acid sequence isolated from the first and at least the second type of malignancy, (3) determining the mean Replikin Count for all data for the first type and at least the second type of malignancy in a given year for which accession numbers are available, (4) selecting at least one year in which the mean Replikin Count for the first malignancy is notably higher, preferably statistically higher, than other years, (5) selecting at least one year in which the mean Replikin Count for at least the second malignancy is notably higher, preferably statistically higher, than other years, (6) identifying the Replikin Peak Gene of each published sequence in the selected year or years for the first malignancy and identifying the Replikin Peak Gene of each published sequence in the selected year or years for the second malignancy, (7) determining the concentration of Replikin sequences for each identified Replikin Peak Gene, (8) selecting the highest Replikin Count as the representative highest Replikin Count for the first malignancy and for at least the second malignancy, and (9) predicting the first malignancy to have a higher growth rate, replication rate and/or greater lethality than at least the second malignancy if the selected highest Replikin Count of the first malignancy is greater than the highest Replikin Count of at least the second malignancy or predicting the first malignancy to have a lower growth rate, replication rate and/or less lethality than at least the second malignancy if the selected highest Replikin Count of the first malignancy is lower than the highest Replikin Count of at least the second malignancy. In a further embodiment, the predicted lethality of the first and at least second malignancies is a five-year mortality rate.

FIG. 1 illustrates a quantitative relationship between the concentration of Replikin peptides in the Replikin Peak Gene of individual proteins associated with cancer cells of different common human malignancies and five-year mortality rates of the different common human malignancies. Replikin Count was determined from the highest Replikin Count identified in a Replikin Peak Gene of sequences surveyed at www.pubmed.com after initially selecting for further analysis (1) those years in which the mean Replikin Counts were the highest, and from those years, (2) those sequences having the highest mean Replikin Count among all available sequences. The five-year mortality rates are as reported in Brenner, H., “Long-term survival rates of cancer patients achieved by the end of the 20th century: a period analysis,” The Lancet, 360 (Oct. 12, 2002), 1131-1135. The lowest Replikin concentrations are seen in thyroid cancer (15 Replikin sequences per 100 amino acids) and in prostate cancer (20 Replikin sequences per 100 amino acids) and the lowest five-year mortality rates are seen in thyroid cancer (2%) and prostate cancer (3%). The highest Replikin concentrations are seen in non-small cell lung carcinoma (250 Replikin sequences per 100 amino acids) and in glioblastoma (324 Replikin sequences per 100 amino acids) and the highest five-year mortality rates are seen in non-small cell lung carcinoma (92%) and glioblastoma (99%). A quantitative progression is seen in relative lethality of each cancer type as compared to the analyzed Replikin Count of each cancer type.

The data for gastric cancer in FIG. 1 demonstrate a higher mortality rate than would be expected from Replikin Count alone as compared to other malignancies. This higher-than-expected mortality rate may be due to a world-wide recognized problem of late detection in gastric cancer. Likewise, the data for urinary bladder cancer and breast cancer in FIG. 1 demonstrate a lower mortality rate than would be expected from Replikin Count alone as compared to other malignancies. The lower-than-expected mortality rate for urinary bladder may be due to art-recognized early detection in bladder cancer. The lower-than-expected mortality rate for breast cancer may similarly be due to increased screening for breast cancer, which results in relatively earlier detection. Breast cancer mortality rate has been decreasing as screening has increased over the past decades.

The data in Table 1 below reflect the data in FIG. 1 and demonstrate a quantitative relationship between Replikin Count in the Replikin Peak Gene of the genome of common types of human cancer and five-year mortality in that cancer as reported in Brenner, H., “Long-term survival rates of cancer patients achieved by the end of the 20th century: a period analysis,” The Lancet, 360 (Oct. 12, 2002), 1131-1135. The discovery of the relation of Replikin sequences to rapid replication as reflected in Table 1 offers a new approach and provides means to inhibit rapid replication and resulting lethality in cancers in animals and humans.

TABLE 1

| | 5-Year Mortality | Highest Replikin |
|---|---|---|
| Human Cancer | of Human Cancer | Count of Replikin |
| Type | Type | Peak Gene |
| Thyroid | 2 | 15 |
| Prostate | 3 | 20 |
| Breast | 11 | 45 |
| Urinary Bladder | 15 | 53 |
| Uterine Corpus | 30 | 24 |
| Uterine Cervix | 34 | 31 |
| Colon | 39 | 28 |
| Ovary | 40 | 60 |
| Oral Cavity | 43 | 53 |
| Lymphocytic | 58 | 128 |
| Leukemia | | |
| Multiple | 70 | 170 |
| Myeloma | | |
| Gastric | 76 | 92 |
| Non-Small Cell | 92 | 250 |
| Lung Carcinoma | | |
| Glioblastoma | 99 | 324 |

Overall, the data in Table 1 and FIG. 1 provide an illustration of a quantitative relationship between Replikin concentration in a given type of cancer and lethality in that type of cancer as compared to the Replikin concentration and lethality in other types of cancer. The data in FIG. 1 also provide further support for a general association between Replikin concentration and lethality within a particular type of cancer (such as, for example, lung cancer) as described in U.S. patent application Ser. No. 12/010,027, filed Jan. 18, 2008.

The association seen in FIG. 1 is surprising to one of ordinary skill in the art because the Replikin Count in these disparate human malignancies is quantitatively related to mean five-year mortality of sufferers of the malignancies even though the malignancies would be expected to have notable differences in disease progression and even though mortality outcomes are significantly dependent upon multiple variables including time of detection and efficacy of disparate treatments. Despite the expected significant differences in time of detection and efficacy of treatment across the population surveyed by Brenner (Lancet 2002), the Replikin concentration in these different human malignancies emerges as a significant variable that quantitatively relates to the mean mortalities reported therein.

### Standard Deviation Measures Change in Rate of Growth

When a mean Replikin Count is determined within a plurality of cells of a malignancy, a larger standard deviation in the mean demonstrates that Replikin Count is changing within the malignancy. These changes within the malignancy point to an increase in the relative rate of growth by demonstrating that certain cells within the malignancy have a relatively higher rate of replication and will be expected to increase the overall rate of growth of the malignancy.

An embodiment of a predictive method of the present invention provides a method of predicting the relative rate of growth of a malignancy comprising: determining the standard deviation of the mean Replikin Count of a plurality of cells of a first malignancy, of a plurality of cells of a metastatic malignancy or a plurality of metastatic malignancies from a patient suffering from a first malignancy, or of a combination of cells of a primary first malignancy and a metastatic malignancy or a plurality of metastatic malignancies of the first malignancy; and determining that the standard deviation of the mean of the first malignancy is relatively larger than the standard deviation of the mean Replikin Count of at least one other malignancy or plurality of malignancies. A standard deviation may be compared between malignancies of the same type or of different types or of metastases within a single patient suffering from a malignancy or between different patients suffering from malignancy. A standard deviation may also be compared between a malignancy, a plurality of malignancies, or a plurality of cells of a malignancy to the standard deviation of a survey of malignancies of the same type, a different type, unknown type, or all types of malignancy.

The standard deviation may be considered relatively large if the standard deviation from the mean Replikin Count is 50% or more of the mean, 40% or more of the mean, 30% or more of the mean, 20% or more of the mean, 10% or more of the mean, 5% or more of the mean, 2% or more of the mean, 1% or more of the mean, 0.5% or more of the mean, or any difference from the mean understood by one of ordinary skill in the art to demonstrate that the population from which the cancer cells in which Replikin Counts have been determined has a changing concentration of Replikin peptides that points to an increase in Replikin Counts within the populations and, as a result, an increase in the rate of growth, rate of replication, or lethality of the cancer cell population.

In an embodiment, a mean Replikin Count with standard deviation may represent a plurality of metastatic cells of the first malignancy. A Replikin Count with standard deviation may also represent a plurality of different metastatic cells of the first malignancy, a plurality of non-metastatic cells of the first malignancy, or a plurality of malignant cells of unknown type from a patient suffering from the first malignancy (or any combination thereof). The Replikin Count of a single cell or a plurality of cells may be compared with a Replikin Count of a single cell or a plurality of cells. A cell or plurality of cells may be predicted to have a greater rate of growth, a greater rate of replication, or a greater lethality than another cell or plurality of cells if the Replikin Count of said cell or malignancy is greater than the mean Replikin Count of the other cell or plurality of cells plus the standard deviation of the mean.

A plurality of cells may also be predicted to have a greater increase in rate of growth, a greater increase in rate of replication, or a greater increase in lethality than another plurality of cells if the standard deviation of the mean Replikin Count of the plurality of cells is greater than the standard deviation of the mean Replikin Count of the other plurality of cells.

Mean Replikin Counts with standard deviation may be determined with a relatively small or large number of cells isolated from a malignancy, may be determined from a relatively small or large number of cells isolated from a plurality of malignancies of the same or similar type, or may be determined from a relatively small or large number of cells isolated from a plurality of cancer types. Replikin Counts with standard deviation may be established from cells isolated from malignancies of the same or similar histological types in a plurality of patients or from malignancies of different histological types in a plurality of patients. A baseline control for Replikin Count and for standard deviation may represent Replikin Counts from a broad collection of malignancies from a broad collection of patients in a particular type of cancer or in all types of cancer. Such controls provide the ordinary skilled artisan with baseline numbers for Replikin Counts and standard deviations in Replikin Counts. Surveys of malignancies are one method of providing such controls.

### Predicting Expansions of Malignancies

One aspect of the present invention provides methods of predicting expansions of malignancies using a Replikin Count Expansion Index. In one embodiment of this aspect of the invention, an expansion of a malignancy (of known or unknown type) or of a metastasis of a malignancy (of known or unknown type) is predicted by (1) determining a mean Replikin Count and a standard deviation from the mean Replikin Count for a plurality of cells of a first malignancy for a first time period in a first anatomic region, (2) determining a Replikin Count of at least one malignant cell (whether primary, metastatic, or unknown) in the body of the patient suffering from said malignancy at a second time period and/or in a second anatomic region or determining a Replikin Count in at least one malignant cell (whether primary, metastatic, or unknown) in the body of a different patient suffering from a second malignancy, and (3) predicting an expansion of the malignancy at the second time period and/or in the second anatomic region or the second malignancy in body of the different patient if the Replikin Count of the at least one malignant cell from the second time period and/or a second anatomic region or the at least one malignant cell from the body of a different patient is greater than one standard deviation of the mean of the Replikin Count of the plurality of cells from the first time period in the first anatomic region.

In another embodiment of the aspect of the invention, an expansion of a malignancy (of known or unknown type) or of a metastasis of a malignancy (of known or unknown type) is predicted by (1) determining a mean Replikin Count and a standard deviation from the mean Replikin Count for a plurality of cells of a first malignancy or a plurality of first malignancies, (2) determining a Replikin Count of at least one malignant cell (whether primary, metastatic, or unknown) in the body of a patient suffering from a second malignancy, and (3) predicting an expansion of the second malignancy if the Replikin Count of the at least one malignant cell from the second malignancy is greater than one standard deviation of the mean of the Replikin Count of the plurality of cells of the first malignancy or the plurality of first malignancies.

In the above-described methods, at least one cell of the malignancy from a second time period and/or second anatomic region or from a second malignancy may be a plurality of cells from the second time period and/or second anatomic region or from the second malignancy. In this case, the Replikin Count of each cell of the plurality of cells from the second time period and/or second anatomic region or from the second malignancy is compared separately to one standard deviation of the mean of the Replikin Count of the plurality of cells from the first time period in the first anatomic region or from the first malignancy.

An expansion of the malignancy isolated in the second time period and/or second anatomic region or in the second malignancy may also be predicted if the number of Replikin Counts of a plurality of cells from the second period and/or second anatomic region or second malignancy that is greater than the mean Replikin Count of the first malignancy plus one standard deviation of the mean is greater than the number of Replikin Counts of said plurality of cells from the second period and/or second anatomic region or second malignancy that is less than the mean Replikin Count of the first malignancy minus one standard deviation of the mean.

An anatomic region of a malignancy or cell of a malignancy is any region of a body of a subject that suffers from a malignancy from which a malignancy, cell of a malignancy, or portion or component of a cell of a malignancy may be identified or isolated. A region may include the entire body of a subject. A region may also be limited to a general region of a body of a subject, such as the thorax, the neck, the knee, the toe, or the leg, may be limited to an organ of a subject, such as the eye, the liver, the skeleton, the blood vessels, or the pancreas of a subject, may be limited to a part of an organ of a subject, may be limited to a particular tissue of a subject, or may be limited to any region of the subject understood by one of skill in the art to provide helpful information in categorizing a malignancy or a cell of a malignancy.

A time period in which a Replikin Count is determined for a malignancy, a plurality of malignancies, a cell of a malignancy, or a plurality of cells of a malignancy may be any time period available to one of skill in the art that is helpful for categorizing a malignancy or the cell of a malignancy. For example, a time period may be prior to surgical removal and another time period may be after surgical removal, a time period may be before, during, or after a therapy such as chemotherapy, radiation, or biological therapy, a time period may be a specific day, a specific week, a specific month, or a specific year of a malignancy in a subject. A time period is any time period that is understood by one of skill in the art to provide helpful information in categorizing, following, diagnosing, or providing prognosis for a malignancy.

The method may also employ a ratio of the number of Replikin Counts that are greater than one standard deviation of the mean divided by the number of Replikin Counts that are less than one standard deviation of the mean. The ratio is called a Replikin Count Expansion Index (RCE Index). Another way to determine the RCE Index is to divide the percent of Replikin Counts in a plurality of cells grouped by time and/or anatomic region that are higher than one standard deviation of the mean by the percent of Replikin Counts that are lower than one standard deviation of the mean. An RCE Index may be used to quantify the future risk of an expansion of a malignancy by tracking Replikin Counts in malignant cells in a patient suffering from malignancy over time.

In determining a RCE Index, the mean Replikin Count of the plurality of cells from the first time period and first anatomic region is considered a control. A control population preferably has a relatively large number of cells with relatively small variability in the Replikin Count of the cells, but any population may be deemed a control when a comparison between the control and a related cell or plurality of cells is desired. A control may be related to the population of cells that is being studied.

For example, if glioma is being studied, the mean Replikin Count with standard deviation of a plurality of cells from the initial biopsy may be compared to Replikin Counts in a plurality of cells from the removed tumor or to Replikin Counts in cells from a metastatic tumor.

A control may also be established from cells isolated from malignancies of the same or similar histological types in a plurality of patients or from malignancies of different histological types in a plurality of patients. A control may represent Replikin Counts from a broad collection of malignancies from a broad collection of patients in a particular type of cancer, in all types of cancer, or in unknown types of cancer. Such controls provide the ordinary skilled artisan with baseline numbers for Replikin Counts and standard deviations in Replikin Counts.

In determining an RCE index, any measure of Replikin concentration may be used. Replikin Count may reflect the concentration of Replikin peptides identified encoded in the genome of a cell. Replikin Count may also reflect the concentration of Replikin peptides identified in the expressed proteins of a cell or in at least one protein or protein fragment of a cell. Replikin Count may also reflect the concentration of Replikin peptides identified in a Replikin Peak Gene of a cell.

Any Replikin peptide, Replikin Peak Gene, protein, protein fragment, or nucleic acid sequence encoding any Replikin peptide, Replikin Peak Gene, protein, or protein fragment in a cell predicted by the methods of the invention to be expanding may be used for diagnostic, therapeutic, and/or preventive purposes. Further, a vaccine may be manufactured by identifying a portion of the structure or genome of a cell predicted to expand in population and using that portion in a vaccine composition.

Methods of the invention also provide methods of predicting a decrease in aggressiveness and/or lethality of a malignancy and/or predicting a contraction of a malignancy wherein a Replikin Count of at least one cell of a malignancy from a second time period and/or second anatomic region is less than one standard deviation of the mean of the Replikin Count of a plurality of cells from a first time period and first anatomic region. A decrease in aggressiveness may also be predicted where the number of Replikin Counts of a plurality of cells from a second time period and/or a second anatomic region that are greater than one standard deviation of the mean is less than the number of Replikin Counts less than one standard deviation of the mean. A decrease or contraction is predicted if the ratio of the Replikin Count Expansion Index is less than one.

When a population contains cells with Replikin Counts above one standard deviation of the mean of a control and does not contain cells with Replikin Counts below one standard deviation of the mean of the control, the ratio of the RCE Index is considered to have a denominator of one to avoid an index of infinity.

In determining a Replikin Count Expansion Index, Replikin Counts from Replikin Peak Genes may be analyzed from anatomic regions in a given time period (such as at initial biopsies or removal of tumors). Within a patient at a particular time, there may be a range of values. The ordinary skilled artisan may select a mean Replikin Count as a control from the range of values.

When comparing the Replikin Count of an individual cell or related group of cells to a control, all Replikin Count values within the group of related cells that fall within one standard deviation of the mean may be treated as a group. Additionally, all values that fall outside the range of one standard deviation from the mean may be treated as two outlying groups. A first group is the group of Replikin Counts that are greater than the mean plus one standard deviation. A second group is the group of Replikin Counts that are less than the mean minus one standard deviation. Because higher Replikin Counts are associated with an expansion of the malignancy and lower Replikin Counts are associated with a decrease in the rate of growth of the malignancy, the ratio of the percent of isolates having Replikin Counts above mean plus one standard deviation to the percent of isolates having Replikin Counts below the mean minus one standard deviation provides a quantitative index of the viability and expansion of the malignancy. The index provides a snapshot of current status of the cancer cell population and the propensity for change in that population. If the ratio is greater than one, the RCE Index predicts an expanding population. If the ratio is less than one, the RCE Index predicts a reduction in growth of the population.

### Conserved Replikin Peptides in Cancer Provide Diagnostic and Therapeutic Targets

Replikin sequences have been observed to be conserved in human cancers generally (and in many pathogenic organisms and viruses) and an increase in concentration of expressed proteins containing Replikin sequences has been observed in cancer as replication increases. See, e.g., discussion of malignin production in a range of cancer types in U.S. application Ser. No. 12/010,027, filed Jan. 18, 2008. In viruses and trypanosomes, cycles of a comparable excess in the Replikin Count have also been related quantitatively to lethality in Taura Syndrome virus in shrimp as well as influenza H5N1 virus in birds and humans, Pl. falciparum malaria in humans, and other human viruses. See, e.g., U.S. application Ser. No. 12/108,458, filed Apr. 23, 2008, U.S. application Ser. No. 12/010,027, filed Jan. 18, 2008, and U.S. Provisional Appln. Ser. No. 61/054,010, filed May 16, 2008. This quantitative relationship has successfully been used to predict differences in lethality. See, e.g., U.S. application Ser. No. 12/108,458, filed Apr. 23, 2008, U.S. application Ser. No. 12/010,027, filed Jan. 18, 2008, and U.S. Provisional Appln. Ser. No. 61/054,010, filed May 16, 2008. Likewise, concentration of Replikin sequences in viral genomes has been shown to increase prior to strain-specific outbreaks of the viruses and increased mortality has been associated with increases in Replikin Count in SARS coronavirus, in influenza, in H5N1 bird flu, and in many other viral and non-viral pathogens. See id. As such, the findings in both infectious diseases and cancer suggest that regardless of the vector and the host, Replikin sequences in the genome are quantitative markers of the degree of lethality produced in the host. Replikin sequences, however, have not been associated in growth, replication, and lethality in malignancies of different types or of unknown types or across malignancies that do not share specific histology or type.

Additionally, Replikin peptide sequences by themselves have been demonstrated to be non-toxic when administered to animals for the purpose of stimulating the immune system. See, e.g., Examples 6 and 7 of U.S. application Ser. No. 11/355,120, filed Feb. 16, 2006. Further, a specific Replikin peptide vaccine was found to be 91% protective against lethal Taura Syndrome virus when administered to shrimp, see U.S. application Ser. No. 12/108,458, filed Apr. 23, 2008, and a vaccine containing a combination of twelve specific Replikin peptides from a low-pathogenic strain of H5N1 influenza virus was found to provide a protective effect and to block both infection and excretion of virus in vaccinated chickens subjected to challenge with the same strain of low-pathogenic H5N1. See U.S. application Ser. No. 12/429,044, filed Apr. 23, 2009.

In cancer, vaccines containing Replikin sequences are likewise effective. For example, peptide sequences from brain cancer, breast cancer, and lymphomas that contain a Replikin sequence each induced production of antimalignin antibodies when administered to animals. The antimalignin antibodies that are produced 1) are demonstrated to be cytotoxic to cancer cells in low doses (picomoles per cell), 2) are demonstrated to increase in concentration in the serum of healthy humans as age and risk of cancer increase, and 3) are quantifiable in serum as an approved test for the early detection of first occurrence and recurrence of malignancies (AMAS® test available from Oncolab, Inc., Boston, Mass.). See, e.g., U.S. Pat. No. 7,381,411, issued Jun. 3, 2008, incorporated by reference herein in its entirety.

Replikin peptide vaccines may be produced by sequencing cancer cell proteins or expressed cancer cell genes, or identifying specific Replikin peptide sequences in the expressed cell proteins, and using the identified Replikin peptide sequences as a basis for production of synthetic cancer vaccines comprising the identified Replikin peptide sequences (service available through Replikins LLC, Boston, Mass.). Concerning cancer vaccines, Replikin peptide sequences are preferred as vaccines over DNA-based vaccines because of the recent demonstration in C. elegans that DNA can be incorporated into the genome of a vaccinated subject and may be reproduced in later generations with unknown consequences.

An embodiment of one aspect of the present invention provides an isolated or synthesized Replikin peptide or Replikin peptides or Replikin Peak Gene or Peak Genes for diagnostic, therapeutic, and/or preventive purposes identified in a malignancy that is predicted to have a higher rate of growth or rate of replication or greater lethality than a second malignancy or than a plurality of other malignancies. In a non-limiting embodiment of an aspect of the present invention, a Replikin peptide is identified in the segment of the genome of the malignancy, of the protein of the malignancy, or of the protein fragment of the malignancy that has the highest concentration of continuous, non-interrupted and/or overlapping Replikin sequences when compared to other segments of the genome of the malignancy, other proteins of the malignancy, or other protein fragments of the malignancy. Another embodiment of one aspect of the invention provides an isolated or synthesized Replikin Peak Gene identified in a malignancy that is predicted to have a higher replication rate or greater lethality than a second malignancy or than a plurality of malignancies. The Replikin Peak Gene of the invention may be used for diagnostic, therapeutic, preventive, and predictive purposes.

Another embodiment of an aspect of the invention provides Replikin peptides or Replikin Peak Genes for diagnostic, therapeutic, and/or preventive purposes identified in a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, or a glioblastoma malignancy.

### Replikins in Diagnostics and Therapies

Identified Replikin peptides and Replikin Peak Genes are also useful where a malignancy is predicted via analysis of Replikin concentration to have a relatively higher or lower rate of replication and/or a greater or lower lethality than another malignancy or wherein a primary malignancy is predicted via analysis of Replikin concentration to have a relatively higher or lower rate of replication or relatively greater or lower lethality than its metastases.

One embodiment of an aspect of the invention provides a method of making a composition comprising at least one component of at least one first malignancy where the first malignancy is predicted according to methods described herein to have a relative rate of growth, replication, or lethality that is greater or less than a second malignancy. The at least one component may comprise any antigenic portion of a cell of the malignancy. The component may likewise comprise at least one Replikin peptide identified in the malignancy or at least one Replikin Peak Gene identified in the malignancy or a combination of Replikin peptides and/or Replikin Peak Genes. A Replikin peptide or Replikin Peak Gene may be isolated from a malignancy predicted to have a greater or lower rate of growth, replication, or lethality. A Replikin peptide or Replikin Peak Gene may likewise be synthesized.

A composition comprising a component of at least one first malignancy may be an immunogenic composition. The composition may be a vaccine or may be comprised in a vaccine. A vaccine may produce an immune response, an antibody response, and/or a protective effect when administered to a subject. The composition may further be combined with a pharmaceutically acceptable carrier or adjuvant or both.

Replikin peptides identified in a malignancy that is relatively more lethal are useful for diagnostic, therapeutic, and preventive purposes with respect to relatively more lethal malignancies. See, e.g., U.S. patent application Ser. No. 12/010,027, filed Jan. 18, 2008, ¶¶ 211-218. For example, a Replikin peptide or a Replikin Peak Gene identified in a relatively more lethal malignancy is useful as a peptide to stimulate the immune system of a human or animal to produce an immune response against malignancies of the type of cancer in which the Replikin peptide was isolated or against other lethal cancers or to produce antibodies against cancers predicted to have higher lethality. One of ordinary skill in the art will recognize that antibodies against these malignancies are useful for diagnosing malignancies of higher lethality in a subject or are useful as therapies against the malignancy or against malignancies of the same or different type or against recurrence of the malignancy in a patient that has suffered from the malignancy. Antibodies against Replikin peptides or Replikin Peak Genes identified in a relatively more lethal malignancy provide a tool for diagnosing or attacking structures in malignancies that are particularly related to rapid replication and/or lethality in a particular malignancy, in a group of malignancies, or in malignancies generally.

Likewise, Replikin peptides identified in relatively lethal malignancies that are conserved in other malignancies or conserved in relatively lethal malignancies are useful as compounds for diagnostic, therapeutic, and preventive purposes. As such, these conserved Replikin peptides are of use as compounds or in compositions for stimulating the immune system of a subject to produce an immune response, an antibody response, and/or a protective effect in the subject.

Because Replikin sequences and Replikin Peak Gene sequences are chemically defined, the sequences may be synthesized by organic chemistry or biological techniques. Replikin sequences synthesized by organic chemistry may be particularly specific, highly reproducible, and highly reliable as compared to other vaccines and therapies. Chemically defined Replikin sequences are likewise potentially freer from adverse reactions characteristic of biologically derived vaccines and antibodies.

An embodiment of an aspect of the invention further contemplates use of Replikin peptides and/or Replikin Peak Genes as immunogenic compositions and contemplates construction of immunogenic compositions as vaccines, including vaccines that provide an immune response, vaccines that provide a humoral immune response, vaccines that provide an antigenic immune response, and vaccines that provide a protective effect.

An immunogenic composition may comprise a Replikin peptide identified in a malignancy predicted to have a higher replication rate or greater lethality than another malignancy or than a plurality of other malignancies. A Replikin peptide may be identified in the segment of the genome, of the protein, or of the protein fragment of the malignancy that has the highest concentration of continuous non-interrupted and/or overlapping Replikin sequences when compared to other segments of the genome, or proteins, or protein fragments of the malignancy. An immunogenic composition may comprise a Replikin Peak Gene identified in a malignancy predicted to have a higher rate of replication or greater lethality or a Replikin Peak Gene identified separately in a primary malignancy and in a metastasis from a primary malignancy or in a metastasis from an unknown primary source.

An immunogenic composition may be comprised in a vaccine for treatment and/or prevention of a malignancy. The malignancy may be predicted to have a higher rate of replication or greater lethality than another malignancy or than a plurality of other malignancies or may be a primary malignancy predicted to have a lower or higher rate of replication or lower or greater lethality than a metastasis of the same malignancy. A vaccine comprising the immunogenic composition may be for the treatment and/or prevention of a malignancy that is of the same type as a malignancy predicted to have a higher rate of replication or greater lethality than another malignancy or than a plurality of other malignancies.

A Replikin peptide or Replikin Peak Gene identified in a first malignancy may be used for the treatment of the first malignancy or may be used for the treatment of another malignancy wherein the other malignancy is related to first malignancy in origin and/or histology, including a metastasis of the first malignancy. A Replikin peptide or Replikin Peak Gene identified in a first malignancy may also be used for the treatment of another malignancy that is unrelated in origin or histology to the first malignancy, including a malignancy of unknown histology or origin.

A Replikin peptide or Replikin Peak Gene identified in a first malignancy may be used for the manufacture of a medicament for the treatment of a malignancy including a malignancy that is related to the first malignancy or unrelated to the first malignancy by histology or origin or any other relationship known to one of skill in the art now or hereafter.

One embodiment of an aspect of the invention provides a method of treating a malignancy comprising administering to a subject at least one Replikin peptide or at least one Replikin Peak Gene or a combination of both identified in a first malignancy predicted, according to methods of the invention, to have a relative rate of growth that is faster than the relative rate of growth of at least one second malignancy or predicted to have greater lethality.

### Antibodies Against Replikins in Diagnostics and Therapies

An embodiment of one aspect of the invention contemplates and produces antibodies to Replikin peptides and to Replikin Peak Genes of the invention. One embodiment provides an antibody to a Replikin peptide identified in a malignancy that is predicted to have a higher growth or replication rate or greater lethality than another malignancy or than a plurality of other malignancies. The Replikin peptide may be identified in any portion of the genome of a cell of a malignancy or in any expressed protein or protein fragment of a cell. The Replikin peptide may also be identified in a segment of the genome, of the protein, or of the protein fragment of the cell from the malignancy that has the highest concentration of continuous non-interrupted and/or overlapping Replikin sequences when compared to other segments of the genome, or proteins, or protein fragments of the cell from the malignancy. An antibody may also be directed to a Replikin Peak Gene identified in a malignancy predicted to have a higher rate of replication or greater lethality.

An antibody or antibody fragment directed against a Replikin peptide or against a Replikin Peak Gene may be used for diagnostic, therapeutic, and/or preventive purposes in cancer, including any cancer known to one of ordinary skill in the art now and hereafter, which may include a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, a glioblastoma malignancy, or any other malignancy of an animal or a human.

One embodiment of an aspect of the invention provides a method of stimulating the immune system of any animal or human capable of an immune response by administering at least one Replikin peptide or at least one Replikin Peak Gene identified in at least one first malignancy predicted, according to methods of the invention, to have a relative rate of growth that is faster than the relative rate of growth of at least one second malignancy. Another embodiment provides a method of making an antibody or an antibody fragment that binds to at least one Replikin peptide or at least one Replikin Peak Gene identified in at least one first malignancy that has a relative rate of growth faster than the relative rate of growth of at least one second malignancy comprising identifying the at least one Replikin peptide or the at least one Replikin Peak Gene and making an antibody or antibody fragment that binds to the at least one Replikin peptide or the at least one Replikin Peak Gene. One of ordinary skill in the art would know myriad ways of making antibodies or antibody fragments that bind to a Replikin peptide or Replikin Peak Gene that is isolated from a cell of a malignancy or for which an amino acid sequence is known.

### Vaccines, Treatments and Therapeutics

A peptide vaccine of the invention may include a single Replikin peptide sequence or Replikin Peak Gene or may include a plurality of Replikin sequences and/or Replikin Peak Genes observed in particular malignancies. A vaccine may include a conserved Replikin peptide or peptides in combination with a Replikin peptide or Replikin peptides in a particular malignancy or may be based on other Replikin peptide sequences such as UTOPES. See U.S. Pat. No. 7,442,761. Replikin peptides can be synthesized by any method, including chemical synthesis or recombinant gene technology, and may include non-Replikin sequences. Vaccine compositions of the invention may also contain a pharmaceutically acceptable carrier and/or adjuvant.

The vaccines of the present invention can be administered alone or in combination with chemotherapies, hormone therapies or other anti-cancer therapies and/or treatments. The vaccine of the present invention may be administered to any animal capable of producing antibodies in an immune response or to any animal capable of producing a humoral response, a protective effect, or any immune or immune-like response. For example, the vaccine of the present invention may be administered to a rabbit, a chicken, a pig, a human, or any other animal capable of producing an immune response and/or antibodies in response to an antigen. Because of the universal nature of Replikin sequences, a vaccine of the invention may be directed at a range of malignancies.

The Replikin peptides of the invention, alone or in various combinations are administered to a subject by any manner known to one of ordinary skill in the art including by intravenous or intramuscular injection, ocular swab or spray, nasal spray and/or inhalation spray, or any other method of administration in order to stimulate the immune system of the subject to produce an immune response. Generally the dosage of peptides is in the range of from about 0.1 μg to about 10 mg, about 10 μg to about 1 mg, and about 50 μg to about 500 μg. The skilled practitioner can readily determine the dosage and number of doses needed to produce an effective immune response.

In another aspect of the invention, isolated Replikin peptides may be used to generate antibodies, which may be used, for example to provide passive immunity in an individual. See, e.g., U.S. application Ser. No. 11/355,120, filed Feb. 16, 2006 and U.S. application Ser. No. 12/010,027, filed Jan. 18, 2008 (each incorporated herein by reference in their entirety).

Anti-Sense Nucleic Acids and siRNA

An embodiment of one aspect of the invention further contemplates a nucleic acid sequence that is antisense to a nucleic acid that encodes for a Replikin peptide or a Replikin Peak Gene identified in a first malignancy predicted to have a higher rate of growth or replication or a greater lethality. Nucleic acid sequences include, for example, one or more small interfering nucleic acid sequences that interfere with a nucleic acid sequence that is 50%, 60%, 70%, 80%, or 90% or more homologous with a nucleic acid that encodes for a Replikin peptide or a Replikin Peak Gene identified in a first malignancy predicted to have a higher rate of growth or replication or a greater lethality, or is 50%, 60%, 70%, 80%, or 90% or more homologous with a nucleic acid that is antisense to a nucleic acid that encodes for a Replikin peptide or a Replikin Peak Gene identified in a first malignancy predicted to have a higher rate of growth or replication or a greater lethality.

Such nucleotide sequences may be used in hybridization assays of biopsied tissue or blood, e.g., Southern or Northern analysis, including in situ hybridization assays, to diagnose the presence of a particular organism in a tissue sample or an environmental sample, for example. The present invention also contemplates kits containing antibodies specific for particular Replikins that are present in a particular pathogen of interest, or containing nucleic acid molecules (sense or antisense) that hybridize specifically to a particular Replikin, and optionally, various buffers and/or reagents needed for diagnosis.

Also within the scope of the invention are oligoribonucleotide sequences that include antisense RNA and DNA molecules and ribozymes that function to inhibit the translation of Replikin-containing mRNA. Both antisense RNA and DNA molecules and ribozymes may be prepared by any method known in the art. The antisense molecules can be incorporated into a wide variety of vectors for delivery to a subject. The skilled practitioner can readily determine the best route of delivery. Intravenous or intramuscular delivery is one possible method of delivery and is one, among many, routine delivery methods in the art of small molecule delivery. The dosage amount is also readily ascertainable. Dosage may range from 0.01 mg to 10 mg, from 0.1 mg to 5 mg, from 0.5 mg to 2 mg, and from 0.75 mg to 1.25 mg, but is not limited to such ranges.

One embodiment of an aspect of the invention further contemplates antisense nucleic acid molecules that are complementary to a nucleic acid encoding a portion of a cell of a malignancy predicted to have a relative greater rate of growth, replication or lethality. An antisense nucleic acid molecule may be complementary to a nucleotide sequence encoding a Replikin peptide or a Replikin Peak Gene as described herein. A nucleic acid sequence may be anti-sense to a nucleic acid sequence that has been demonstrated to be conserved in a malignancy or generally conserved in a range of malignancies of a particular cancer type or of different cancer types.

The invention also contemplates compositions comprising RNAi-inducing entities used to inhibit replication of a malignancy including small interfering RNA, which is a class of about 10 to about 50, and often about 20 to about 25, nucleotide-long double-stranded RNA molecules. siRNA is involved in the RNA interference pathway, where it interferes with the expression of one or more specific genes such as replication genes of a malignancy including replication genes that comprise at least one Replikin peptide or at least one Replikin Peak Gene as described herein. siRNAs also act in RNAi-related pathways, e.g., as an antireplication mechanism.

An effective amount of an RNAi-inducing entity is delivered to a cell or organism prior to, simultaneously with, or after diagnosis of a malignancy or a metastasis. A dosage may be sufficient to reduce or delay replication of the malignancy or metastasis. Compositions of the invention may comprise a single siRNA species targeted to a target transcript or may comprise a plurality of different siRNA species targeting one or more target transcripts.

The invention contemplates a small interfering nucleic acid sequence that is about 10 to about 50 nucleic acids in length and is 50%, 60%, 70%, 80%, or 90% or more homologous with a nucleic acid that encodes for any portion of at least one Replikin peptide or at least one Replikin Peak Gene, or is 50%, 60%, 70%, 80%, or 90% or more homologous with a nucleic acid that is antisense to a nucleic acid that encodes for any portion of at least one Replikin peptide or at least one Replikin Peak Gene. In a further non-limiting embodiment, the nucleic acid sequence is about 15 to about 30 nucleic acids. In a further non-limiting embodiment, the nucleic acid sequence is about 20 to about 25 nucleic acids. In a further non-limiting embodiment, the nucleic acids sequence is about 21 nucleic acids.

### Prognosis for Individual Malignancies Based on Replikin Count of Malignancy and Survey of Plurality of Malignancies

An individual prognosis for a malignancy may be provided for a patient suffering from the malignancy based on Replikin Count and lethality data from a survey of patients suffering from malignancies of the same type, malignancies of related types, or malignancies in general. For example, a survey may be undertaken in which Replikin Counts of tissues from tumors removed from patients are analyzed and compared with patient outcome.

For example, in one non-limiting embodiment, a Replikin Count Expansion Index may be created from a survey of malignancies. To create a Replikin Count Expansion Index, a mean Replikin Count plus one standard deviation of the mean is determined from a plurality of malignancies and the measured lethality of each malignancy is determined by any method.

A Replikin Count of a malignancy having an unknown lethality may then be compared to the mean Replikin Count plus one standard deviation of the mean. If the Replikin Count of the malignancy having an unknown lethality is greater than the mean Replikin Count plus one standard deviation of the mean, the malignancy of unknown lethality is predicted to have a greater lethality than the mean measured lethality of plurality of malignancies. If the Replikin Count of the malignancy having an unknown lethality is less than the mean Replikin Count minus one standard deviation of the mean, the malignancy of unknown lethality is predicted to have a lower lethality than the mean measured lethality of the plurality of malignancies.

To provide other kinds of precision in the prediction of lethality, a plurality of malignancies may be grouped into a plurality of groups of malignancies having related lethalities or related types. A mean Replikin Count plus one standard deviation of the mean may be determined for some or all of these groups. A Replikin Count of a malignancy having an unknown lethality may then be compared to the mean Replikin Count plus or minus one standard deviation of the mean of a particular group having a related lethality.

In another non-limiting embodiment, regression analysis may be undertaken based on the Replikin Count data and the data of patient outcome. The resulting regression formula may be used to provide a prognosis for a patient suffering from a malignancy wherein a Replikin Count has been determined from the malignancy.

For example, a Replikin Count determined in a lung tumor (or any other malignancy) of a patient suffering from lung cancer (or any other malignancy) may be entered into a formula generated using survey data of Replikin Counts of individual lung tumors (or any other malignancy) and patient outcome from the individual lung tumors (or other malignancies). The formula may provide a prognosis concerning the rate of growth, aggressiveness, or lethality of the lung tumor (or other malignancy) based on the Replikin Count of the lung tumor.

Patient outcome may include but is not limited to five year mortality rate, life expectancy, aggressiveness of growth, rate of growth, rate of replication, rate of decline of lung capacity, etc. Patient outcome may include any measure of patient well-being that may be compared to Replikin Count of a malignancy.

Replikin Count may be any measure of Replikin Count in a malignancy including Replikin Count of the entire genome or of a portion or fragment of the genome, Replikin Count of an expressed protein, Replikin Count of a Replikin Peak Gene, Replikin Count of a protein fragment, Replikin Count of a combination of proteins and/or protein fragments, or mean Replikin Count of a plurality of cells in a malignancy, etc.

In a survey, the Replikin Count measurement having the best correlation with the patient outcome measurement may be used for regression analysis. The greater the size of the survey, the more precision that is expected from the regression analysis. As such, a survey of a plurality of malignancies would be appropriate as a survey. Likewise, a survey of 10 malignancies, 50 malignancies, 100 malignancies, 500 malignancies, 1,000 malignancies, or 10,000 or more malignancies would be appropriate for generating a Replikin Count Expansion Index or a formula from regression analysis.

Within a survey of malignancies, more than one regression analysis may be performed. For example, two regression curves may be generated within one data set from a survey. Two regression curves may be appropriate where a data set contains, for example, both benign and malignant tumors. A regression curve for benign tumors may be quite different from a regression curve for malignant tumors. Likewise, regression curves may be different for different types of malignancies. The closer a regression curve fits to the particular data to which it is applied, the more precision that would be expected in predicting patient outcome from a malignancy of unknown lethality.

For example, benign tumors may be expected to have a growth rate that allows many years of growth of the tumor while a malignant tumor may have a growth rate that allows less years of growth, including tumors that are lethal in days, months, or one or two or more years. Different regression curves that apply to different data sets provide one of ordinary skill in the art with many ways to separate different types of malignancies and many types of curves for predicting lethality. Regression curves may be linear, polynomial, exponential, or any other type of regression curve known to one of skill in the art now or hereafter. One, two, three, or more regression curves may be applied to a single data set based on the understanding of one of ordinary skill in the art.

Health practitioners may employ a formula generated from a Replikin Count Expansion Index or regression analysis, or any other kind of analysis to provide a prognosis for a patient suffering from a malignancy. For example, if a patient is suffering from a lung malignancy, a health practitioner may take a biopsy of the malignancy and have the Replikin Count of a Replikin Peak Gene of a particular protein of the malignancy determined. The health practitioner may then enter the Replikin Count of the Replikin Peak Gene of the particular protein of the lung malignancy into an index or formula generated from a survey based on Replikin Counts of the Replikin Peak Gene of the lung malignancy from which the patient is suffering. The index or formula may provide a prognosis concerning the rate of growth of the malignancy, the aggressiveness of the malignancy, or the life expectancy of the patient suffering from the malignancy. The resulting prognosis may then be provided to the patient suffering from the lung malignancy.

A survey and resulting Replikin Count Expansion Index or regression analysis may be undertaken for any malignancy or any type of malignancy known to one of skill in the art now or hereafter. A survey and resulting Replikin Count Expansion Index or regression analysis may also be done for malignancies generally wherein the Replikin Count of any malignancy may be entered into an index or formula to provide a prognosis concerning the growth or lethality of the malignancy.

A survey of a specific type of malignancy will provide a more defined prognosis for a patient suffering from that specific malignancy. Further, a survey of a specific type of malignancy at a specific stage of growth may provide an even more defined prognosis for a patient suffering from that specific malignancy at that specific stage of growth.

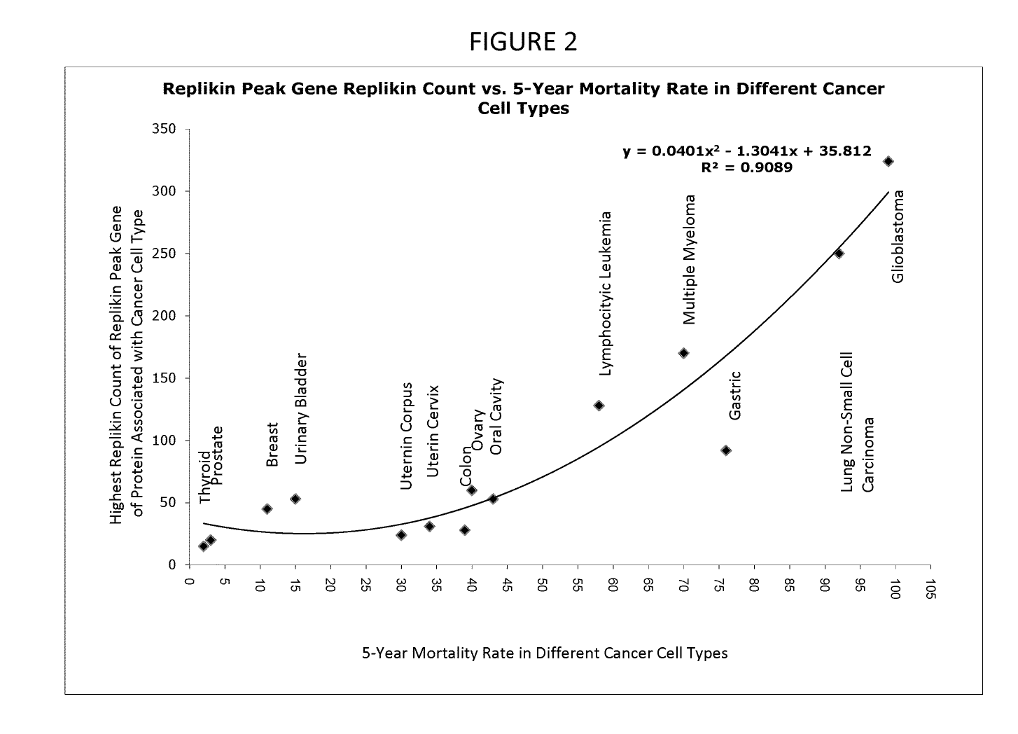

FIG. 2 provides a regression analysis of the data in FIG. 1, which compares the highest Replikin Count of a Replikin Peak Gene of various common human cancers with the five-year mortality rate of the cancer as provided in Brenner, H., The Lancet, 360 (Oct. 12, 2002), 1131-1135. The regression formula provided in FIG. 2 is y=0.0401x²±1.3041x+35.812 with an r² value of 0.9089. This regression formula may be used to provide a prediction of the relative five year mortality of a malignancy based on the highest Replikin Count of a Replikin Peak Gene in a representative malignancy of a known type of malignancy. By introducing the Replikin Count of the Replikin Peak Gene of the representative malignancy into the y value of the regression formula, the resulting x value will provide a predicted five year mortality rate. Any other kind of regression analysis may be applied to the data in FIG. 2.

The process of creating a regression formula for a given type of malignancy or for a range of malignancies may be undertaken from a survey of any plurality of malignancies. Once baseline data is provided by the survey, the formula may be used to provide a patient suffering from a malignancy a prognosis based on a prediction of the rate of growth, rate of replication, aggressiveness, or lethality of a malignancy.

A prognosis based on a Replikin Count Expansion Index or based on a regression analysis of Replikin Count and measured lethality may be provided for any malignancy including but not limited to, a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, or a glioblastoma malignancy.

The efficacy of histopathologically-determined degrees of malignancy may also be quantified by comparing the histopathological degree of malignancy given to a tumor with a quantitative prognosis determined from a Replikin Count of a malignancy entered into a Replikin Count Expansion Index or a formula developed from regression analysis of Replikin Counts of malignancies of the same or related types or from malignancies of all types.

A kit for providing a prognosis to a patient suffering from a malignancy is contemplated comprising a Replikin Count Expansion Index or a formula for determining prognosis based on Replikin Count of the malignancy. Such a kit may be provided to laboratories and practitioners so that prognosis based on Replikin sequence analysis of individual malignancies may be provided directly from the practitioner or laboratory to the patient.

### Example 1

### Analysis of Replikin Count in Replikin Peak Gene of Common Human Cancer Types

Applicants analyzed publicly available sequences of the common human malignancies listed in Table 1 as those sequences had been published at www.pubmed.com. Using REPLIKINSFORECAST® software service available through Replikins LLC (Boston, Mass.), Applicants determined the Replikin Count for each accession number with a sequence isolated from the listed malignancies (i.e., all accession numbers containing sequences published as isolated from prostate cancers, thyroid cancers, breast cancers, urinary bladder cancers, etc.) Applicants then determined mean Replikin Count for all data for each listed common human malignancy in a given year for which accession numbers were available.

For those years in which mean Replikin Count was notably higher than other years, Applicants identified the Replikin Peak Gene of each published sequence. The Replikin Peak Gene is defined as the segment of the reported genome, reported protein, or reported protein fragment containing the highest number of continuous and/or overlapping Replikin sequences per 100 amino acids (Replikin Count) for each identified Replikin Peak Gene. For the purpose of the Replikin Count, each Replikin sequence is defined as a peptide sequence consisting of 7 to 50 amino acids (or a nucleic acid sequence encoding a peptide sequence consisting of 7 to 50 amino acids) and having (or encoding a sequence having) (1) at least one lysine residue located at a first terminus of the sequence and at least one lysine residue or at least one histidine residue located at a second terminus of the sequence, (2) a first lysine residue located six to ten residues from a second lysine residue, (3) at least one histidine residue, and (4) at least 6% lysine residues.

For each identified Replikin Peak Gene, Applicants determined the concentration of Replikin sequences by determining the number of Replikin sequences per 100 amino acids (Replikin Count) in the identified Replikin Peak Gene. From the Replikin Count data for each identified Replikin Peak Gene, Applicants selected the highest Replikin Count as the representative highest Replikin Count for a given common human malignancy.

Applicants then compared the representative highest Replikin Count for each common human malignancy to the percent five-year mortality rate reported at Brenner, H., “Long-term survival rates of cancer patients achieved by the end of the 20th century: a period analysis,” The Lancet, 360 (Oct. 12, 2002), 1131-1135. The comparative data are shown in Table 1 and the comparison is graphically presented in FIG. 1. FIG. 1 graphically demonstrates a quantitative relationship between highest Replikin Count in a malignancy and reported five-year mortality rate.

### Example 2

### Prediction of Relative Mortality in Human Malignancies

The Replikin Count of an identified Replikin Peak Gene is analyzed following sequencing of some or all of the genome of at least a first and a second malignancy or following the sequencing of at least one protein or protein fragment of at least a first and a second malignancy. The Replikin Count of the Replikin Peak Gene of the first malignancy is compared to the Replikin Count of the Replikin Peak Gene of at least the second malignancy. If the Replikin Count is determined to be higher than at least the second malignancy, the first malignancy is predicted to have a relatively higher rate of replication and/or greater lethality than at least the second malignancy. If the Replikin Count is determined to be lower than at least the second malignancy, the first malignancy is predicted to have a relatively lower rate of replication and/or greater lethality than at least the second malignancy.

### Example 3

### Prediction of Relative Rate of Increase of Growth Rate of Malignancy

The relative rate of change in the rate of growth of a metastasis of a malignancy is predicted by comparing the standard deviation of the mean Replikin Count of the genome, a genome fragment, all expressed proteins, an expressed protein, or a protein fragment of a plurality of cells of the primary malignancy to the standard deviation of the mean Replikin Count of the genome, a genome fragment, all expressed proteins, an expressed protein, or a protein fragment of a plurality of cells of a metastasis of the malignancy. The mean Replikin Count of a metastasis may likewise be compared to a mean Replikin Count of a plurality of metastases from patients having a similar malignancy or from a broad range of malignancies generally. If the standard deviation of the mean Replikin Count in a plurality of cells of the primary malignancy is greater than the standard deviation of the mean Replikin Count in a plurality of cells of the metastasis of the malignancy (or of a mean Replikin Count of a plurality of metastases from patients having a similar malignancy or from a broad range of malignancies generally), the metastasis of the malignancy is predicted to experience a decrease in the relative rate of change in the rate of growth as compared to the primary malignancy. On the other hand, if the standard deviation of the mean Replikin Count in a plurality of cells of the primary malignancy is less than the standard deviation of the mean Replikin Count in a plurality of cells of the metastasis of the malignancy (or of a mean Replikin Count of a plurality of metastases from patients having a similar malignancy or from a broad range of malignancies generally), the metastasis of the malignancy is predicted to experience an increase in the relative rate of change in the rate of growth as compared to the primary malignancy.

### Example 4

### Replikin Count Expansion Index in Primary and Metastatic Malignancy Over Time

Replikin Count of a genome or genome fragment or expressed proteins, an expressed protein, or protein fragment of a plurality of cells from a malignancy are determined. Replikin Counts from cells from a specific time and/or specific anatomical region of the malignancy are grouped and a mean Replikin Count with standard deviation is determined for each group. The cells may be from the primary malignancy or a metastasis of the malignancy, or may be of unknown origin within the body of the patient suffering from the malignancy.

Replikin Count of a genome or genome fragment or expressed proteins, an expressed protein, or protein fragment of at least one cell of a metastasis of the malignancy (or from a malignant cell of unknown origin or type within the body of the patient suffering from the malignancy) from a second time and/or second anatomical region in the patient suffering from the malignancy is determined. If Replikin Count is determined for more than one cell, a mean Replikin Count with standard deviation is determined.

The time period and/or anatomical region having the largest number of cells or the least variability among Replikin Count in cells (or both) is chosen as a control against which other Replikin Counts are analyzed. The Replikin Count for each individual cell in a given anatomical region at a particular time is compared to one standard deviation from the mean Replikin Count for all cells from the control region. Within each region, the number of Replikin Counts greater than one standard deviation of the mean and the number of Replikin Counts less than one standard deviation of the mean is determined. For each region at each time period, the percent of Replikin Counts greater than one standard deviation of the mean is then divided by the percent of Replikin Counts less than one standard deviation of the mean to provide a ratio, or Replikin Count Expansion (RCE) Index. In anatomical regions having an RCE Index of greater than one, an expansion of the rate of growth of the malignancy is predicted. In anatomical regions having an RCE Index of less than one, a contraction of the rate of growth of the malignancy is predicted.

In anatomical regions wherein the rate of growth of a malignancy is predicted to expand, a Replikin Peak Gene is identified in a cell having a Replikin Count that is higher than the mean Replikin Count for the anatomical region. The Replikin Peak Gene and/or a Replikin peptide (or plurality of Replikin peptides) within the Replikin Peak Gene is selected as an immunogenic compound for diagnostic and/or therapeutic purposes. A vaccine against the expanding malignancy is manufactured comprising the immunogenic compound. The vaccine is administered to mitigate the expanding malignancy.

### Example 5

### Determination of Replikin Count or Replikin Peak Gene in 1999 Isolate of Glioblastoma Topoisomerase in Rat and Development of Glioblastoma Vaccine in Rat Model

Accession numbers of amino acid sequences of glioblastoma malignancies in rats were queried at www.pubmed.com. Replikin Count data from the queried accession numbers provided predictions of the relative lethality of each malignancy. The highest Replikin Count in a Replikin Peak Gene in an amino acid sequence from rat glioblastoma was identified in a 1999 topoisomerase isolate of glioblastoma in Accession Number Q9WUL0 taken from a study of the C6 glioblastoma cell line in Norway rats. Among the queried accession numbers, the topoisomerase in Accession Number Q9WUL0 had the highest Replikin Count in a Replikin Peak Gene with a Replikin Count of 316 Replikin sequences per 100 amino acids within the Replikin Peak Gene.

Accession Number Q9WUL0 contains the following amino acid sequence:

(SEQ ID NO: 1)

m¹ s² g³ d⁴ h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸
k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴
e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³ i¹⁰⁴
k¹⁰⁵ k¹⁰⁶ e¹⁰⁷ k¹⁰⁸ e¹⁰⁹ n¹¹⁰ g¹¹¹ f¹¹² s¹¹³ s¹¹⁴ p¹¹⁵ p¹¹⁶ r¹¹⁷ i¹¹⁸ k¹¹⁹ d¹²⁰ e¹²¹ p¹²² e¹²³ d¹²⁴
d¹²⁵ g¹²⁶ y¹²⁷ f¹²⁸ a¹²⁹ p¹³⁰ p¹³¹ k¹³² e¹³³ d¹³⁴ i¹³⁵ k¹³⁶ p¹³⁷ l¹³⁸ k¹³⁹ r¹⁴⁰ p¹⁴¹ r¹⁴² d¹⁴³ e¹⁴⁴
d¹⁴⁵ d¹⁴⁶ a¹⁴⁷ d¹⁴⁸ y¹⁴⁹ k¹⁵⁰ p¹⁵¹ k¹⁵² k¹⁵³ i¹⁵⁴ k¹⁵⁵ t¹⁵⁶ e¹⁵⁷ d¹⁵⁸ i¹⁵⁹ k¹⁶⁰ k¹⁶¹ e¹⁶² k¹⁶³ k¹⁶⁴
r¹⁶⁵ k¹⁶⁶ l¹⁶⁷ e¹⁶⁸ e¹⁶⁹ e¹⁷⁰ e¹⁷¹ d¹⁷² g¹⁷³ k¹⁷⁴ l¹⁷⁵ k¹⁷⁶ k¹⁷⁷ p¹⁷⁸ k¹⁷⁹ n¹⁸⁰ k¹⁸¹ d¹⁸² k¹⁸³ d¹⁸⁴
k¹⁸⁵ k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵ k¹⁹⁶ a¹⁹⁷ k¹⁹⁸ k¹⁹⁹ e²⁰⁰ e²⁰¹ e²⁰² q²⁰³ k²⁰⁴
w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷ k²¹⁸ w²¹⁹ k²²⁰ f²²¹ l²²² e²²³ h²²⁴
k²²⁵ g²²⁶ p²²⁷ v²²⁸ f²²⁹ a²³⁰ p²³¹ p²³² y²³³ e²³⁴ p²³⁵ l²³⁶ p²³⁷ e²³⁸ g²³⁹ v²⁴⁰ k²⁴¹ f²⁴² y²⁴³ y²⁴⁴
d²⁴⁵ g²⁴⁶ k²⁴⁷ v²⁴⁸ m²⁴⁹ k²⁵⁰ l²⁵¹ s²⁵² p²⁵³ k²⁵⁴ a²⁵⁵ e²⁵⁶ e²⁵⁷ v²⁵⁸ a²⁵⁹ t²⁶⁰ f²⁶¹ f²⁶² a²⁶³ k²⁶⁴
m²⁶⁵ l²⁶⁶ d²⁶⁷ h²⁶⁸ e²⁶⁹ y²⁷⁰ t²⁷¹ t²⁷² k²⁷³ e²⁷⁴ i²⁷⁵ f²⁷⁶ r²⁷⁷ k²⁷⁸ n²⁷⁹ f²⁸⁰ f²⁸¹ k²⁸² d²⁸³ w²⁸⁴
r²⁸⁵ k²⁸⁶ e²⁸⁷ m²⁸⁸ t²⁸⁹ n²⁹⁰ d²⁹¹ e²⁹² k²⁹³ n²⁹⁴ t²⁹⁵ i²⁹⁶ t²⁹⁷ n²⁹⁸ l²⁹⁹ s³⁰⁰ k³⁰¹ c³⁰² d³⁰³ f³⁰⁴
t³⁰⁵ q³⁰⁶ m³⁰⁷ s³⁰⁸ q³⁰⁹ y³¹⁰ f³¹¹ k³¹² a³¹³ q³¹⁴ s³¹⁵ e³¹⁶ a³¹⁷ r³¹⁸ k³¹⁹ q³²⁰ m³²¹ s³²² k³²³ e³²⁴
e³²⁵ k³²⁶ l³²⁷ k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴
m³⁴⁵ d³⁴⁶ n³⁴⁷ h³⁴⁸ r³⁴⁹ e³⁵⁰ r³⁵¹ i³⁵² a³⁵³ n³⁵⁴ f³⁵⁵ k³⁵⁶ i³⁵⁷ e³⁵⁸ p³⁵⁹ p³⁶⁰ g³⁶¹ l³⁶² f³⁶³ r³⁶⁴
g³⁶⁵ r³⁶⁶ g³⁶⁷ n³⁶⁸ h³⁶⁹ p³⁷⁰ k³⁷¹ m³⁷² g³⁷³ m³⁷⁴ l³⁷⁵ k³⁷⁶ r³⁷⁷ r³⁷⁸ i³⁷⁹ m³⁸⁰ p³⁸¹ e³⁸² d³⁸³ i³⁸⁴
i³⁸⁵ i³⁸⁶ n³⁸⁷ c³⁸⁸ s³⁸⁹ k³⁹⁰ d³⁹¹ a³⁹² k³⁹³ v³⁹⁴ p³⁹⁵ s³⁹⁶ p³⁹⁷ p³⁹⁸ p³⁹⁹ g⁴⁰⁰ h⁴⁰¹ k⁴⁰² w⁴⁰³ k⁴⁰⁴
e⁴⁰⁵ v⁴⁰⁶ r⁴⁰⁷ h⁴⁰⁸ d⁴⁰⁹ n⁴¹⁰ k⁴¹¹ v⁴¹² t⁴¹³ w⁴¹⁴ l⁴¹⁵ v⁴¹⁶ s⁴¹⁷ w⁴¹⁸ t⁴¹⁹ e⁴²⁰ n⁴²¹ i⁴²² q⁴²³ g⁴²⁴
s⁴²⁵ i⁴²⁶ k⁴²⁷ y⁴²⁸ i⁴²⁹ m⁴³⁰ l⁴³¹ n⁴³² p⁴³³ s⁴³⁴ s⁴³⁵ r⁴³⁶ i⁴³⁷ k⁴³⁸ g⁴³⁹ e⁴⁴⁰ k⁴⁴¹ d⁴⁴² w⁴⁴³ q⁴⁴⁴
k⁴⁴⁵ y⁴⁴⁶ e⁴⁴⁷ t⁴⁴⁸ a⁴⁴⁹ r⁴⁵⁰ r⁴⁵¹ l⁴⁵² k⁴⁵³ k⁴⁵⁴ c⁴⁵⁵ v⁴⁵⁶ d⁴⁵⁷ k⁴⁵⁸ i⁴⁵⁹ r⁴⁶⁰ n⁴⁶¹ q⁴⁶² y⁴⁶³ r⁴⁶⁴
e⁴⁶⁵ d⁴⁶⁶ w⁴⁶⁷ k⁴⁶⁸ s⁴⁶⁹ k⁴⁷⁰ e⁴⁷¹ m⁴⁷² k⁴⁷³ v⁴⁷⁴ r⁴⁷⁵ q⁴⁷⁶ r⁴⁷⁷ a⁴⁷⁸ v⁴⁷⁹ a⁴⁸⁰ l⁴⁸¹ y⁴⁸² f⁴⁸³ i⁴⁸⁴
d⁴⁸⁵ k⁴⁸⁶ l⁴⁸⁷ a⁴⁸⁸ l⁴⁸⁹ r⁴⁹⁰ a⁴⁹¹ g⁴⁹² n⁴⁹³ e⁴⁹⁴ k⁴⁹⁵ e⁴⁹⁶ e⁴⁹⁷ g⁴⁹⁸ e⁴⁹⁹ t⁵⁰⁰ a⁵⁰¹ d⁵⁰² t⁵⁰³ v⁵⁰⁴
g⁵⁰⁵ c⁵⁰⁶ c⁵⁰⁷ s⁵⁰⁸ l⁵⁰⁹ r⁵¹⁰ v⁵¹¹ e⁵¹² h⁵¹³ i⁵¹⁴ n⁵¹⁵ l⁵¹⁶ h⁵¹⁷ p⁵¹⁸ e⁵¹⁹ l⁵²⁰ d⁵²¹ g⁵²² q⁵²³ e⁵²⁴
y⁵²⁵ v⁵²⁶ v⁵²⁷ e⁵²⁸ f⁵²⁹ d⁵³⁰ f⁵³¹ p⁵³² g⁵³³ k⁵³⁴ d⁵³⁵ s⁵³⁶ i⁵³⁷ r⁵³⁸ y⁵³⁹ y⁵⁴⁰ n⁵⁴¹ k⁵⁴² v⁵⁴³ p⁵⁴⁴
v⁵⁴⁵ e⁵⁴⁶ k⁵⁴⁷ r⁵⁴⁸ v⁵⁴⁹ f⁵⁵⁰ k⁵⁵¹ n⁵⁵² l⁵⁵³ q⁵⁵⁴ l⁵⁵⁵ f⁵⁵⁶ m⁵⁵⁷ e⁵⁵⁸ n⁵⁵⁹ k⁵⁶⁰ q⁵⁶¹ p⁵⁶² e⁵⁶³ d⁵⁶⁴
d⁵⁶⁵ l⁵⁶⁶ f⁵⁶⁷ d⁵⁶⁸ r⁵⁶⁹ l⁵⁷⁰ n⁵⁷¹ t⁵⁷² g⁵⁷³ i⁵⁷⁴ l⁵⁷⁵ n⁵⁷⁶ k⁵⁷⁷ h⁵⁷⁸ l⁵⁷⁹ q⁵⁸⁰ d⁵⁸¹ l⁵⁸² m⁵⁸³ e⁵⁸⁴
g⁵⁸⁵ l⁵⁸⁶ t⁵⁸⁷ a⁵⁸⁸ k⁵⁸⁹ v⁵⁹⁰ f⁵⁹¹ r⁵⁹² t⁵⁹³ y⁵⁹⁴ n⁵⁹⁵ a⁵⁹⁶ s⁵⁹⁷ i⁵⁹⁸ t⁵⁹⁹ l⁶⁰⁰ q⁶⁰¹ q⁶⁰² q⁶⁰³ l⁶⁰⁴
k⁶⁰⁵ e⁶⁰⁶ l⁶⁰⁷ t⁶⁰⁸ a⁶⁰⁹ p⁶¹⁰ d⁶¹¹ e⁶¹² n⁶¹³ v⁶¹⁴ p⁶¹⁵ a⁶¹⁶ k⁶¹⁷ i⁶¹⁸ l⁶¹⁹ s⁶²⁰ y⁶²¹ n⁶²² r⁶²³ a⁶²⁴
n⁶²⁵ r⁶²⁶ a⁶²⁷ v⁶²⁸ a⁶²⁹ i⁶³⁰ l⁶³¹ c⁶³² n⁶³³ h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴
s⁶⁴⁵ m⁶⁴⁶ m⁶⁴⁷ n⁶⁴⁸ l⁶⁴⁹ q⁶⁵⁰ s⁶⁵¹ k⁶⁵² i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴
k⁶⁶⁵ d⁶⁶⁶ l⁶⁶⁷ k⁶⁶⁸ s⁶⁶⁹ a⁶⁷⁰ k⁶⁷¹ a⁶⁷² d⁶⁷³ a⁶⁷⁴ k⁶⁷⁵ v⁶⁷⁶ m⁶⁷⁷ k⁶⁷⁸ d⁶⁷⁹ a⁶⁸⁰ k⁶⁸¹ t⁶⁸² k⁶⁸³ k⁶⁸⁴
v⁶⁸⁵ v⁶⁸⁶ e⁶⁸⁷ s⁶⁸⁸ k⁶⁸⁹ k⁶⁹⁰ k⁶⁹¹ a⁶⁹² v⁶⁹³ q⁶⁹⁴ r⁶⁹⁵ l⁶⁹⁶ e⁶⁹⁷ e⁶⁹⁸ q⁶⁹⁹ l⁷⁰⁰ m⁷⁰¹ k⁷⁰² l⁷⁰³ e⁷⁰⁴
v⁷⁰⁵ q⁷⁰⁶ a⁷⁰⁷ t⁷⁰⁸ d⁷⁰⁹ r⁷¹⁰ e⁷¹¹ e⁷¹² n⁷¹³ k⁷¹⁴ q⁷¹⁵ i⁷¹⁶ a⁷¹⁷ l⁷¹⁸ g⁷¹⁹ t⁷²⁰ s⁷²¹ k⁷²² l⁷²³ n⁷²⁴
y⁷²⁵ l⁷²⁶ d⁷²⁷ p⁷²⁸ r⁷²⁹ i⁷³⁰ t⁷³¹ v⁷³² a⁷³³ w⁷³⁴ c⁷³⁵ k⁷³⁶ k⁷³⁷ w⁷³⁸ g⁷³⁹ v⁷⁴⁰ p⁷⁴¹ i⁷⁴² e⁷⁴³ k⁷⁴⁴
i⁷⁴⁵ y⁷⁴⁶ n⁷⁴⁷ k⁷⁴⁸ t⁷⁴⁹ q⁷⁵⁰ r⁷⁵¹ e⁷⁵² k⁷⁵³ f⁷⁵⁴ a⁷⁵⁵ w⁷⁵⁶ a⁷⁵⁷ i⁷⁵⁸ d⁷⁵⁹ m⁷⁶⁰ t⁷⁶¹ d⁷⁶² e⁷⁶³ d⁷⁶⁴
y^{765 e}⁷⁶⁶ f⁷⁶⁷.

The following Replikin sequences were identified in the amino terminal of Accession Number Q9WUL0:

(SEQ ID NO: 2)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹

(SEQ ID NO: 3)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹
d³⁰ r³¹ e³² h³³ r³⁴ h³⁵ k³⁶

(SEQ ID NO: 4)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹
d³⁰ r³¹ e³² h³³ r³⁴ h³⁵ k³⁶ e³⁷ h³⁸ k³⁹

(SEQ ID NO: 5)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹
d³⁰ r³¹ e³² h³³ r³⁴ h³⁵ k³⁶ e³⁷ h³⁸ k³⁹ k⁴⁰ d⁴¹ k⁴²

(SEQ ID NO: 6)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹
d³⁰ r³¹ e³² h³³ r³⁴ h³⁵ k³⁶ e³⁷ h³⁸ k³⁹ k⁴⁰ d⁴¹ k⁴² d⁴³ k⁴⁴

(SEQ ID NO: 7)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹
d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸

(SEQ ID NO: 8)

h⁵ l⁶ h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹
d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰

(SEQ ID NO: 9)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹

(SEQ ID NO: 10)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶

(SEQ ID NO: 11)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶ e³⁷ h³⁸ k³⁹

(SEQ ID NO: 12)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶ e³⁷ h³⁸ k³⁹ k⁴⁰ d⁴¹ k⁴²

(SEQ ID NO: 13)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶ e³⁷ h³⁸ k³⁹ k⁴⁰ d⁴¹ k⁴² d⁴³ k⁴⁴

(SEQ ID NO: 14)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸

(SEQ ID NO: 15)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰

(SEQ ID NO: 16)

h⁷ n⁸ d⁹ s¹⁰ q¹¹ i¹² e¹³ a¹⁴ d¹⁵ f¹⁶ r¹⁷ l¹⁸ n¹⁹ d²⁰ s²¹ h²² k²³ h²⁴ k²⁵ d²⁶ k²⁷ h²⁸ k²⁹ d³⁰ r³¹
e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷

(SEQ ID NO: 17)

h²² k²³ h²⁴ k²⁵ d²⁶ k²⁷ h²⁸ k²⁹

(SEQ ID NO: 18)

h²² k²³ h²⁴ k²⁵ d²⁶ k²⁷ h²⁸ k²⁹ d³⁰ r³¹ e³² h³³ r³⁴ h³⁵ k³⁶

(SEQ ID NO: 19)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹

(SEQ ID NO: 20)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴²

(SEQ ID NO: 21)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴

(SEQ ID NO: 22)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸

(SEQ ID NO: 23)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰

(SEQ ID NO: 24)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰ h⁵¹ s⁵² n⁵³ s⁵⁴ e⁵⁵ h⁵⁶ k⁵⁷

(SEQ ID NO: 25)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴

(SEQ ID NO: 26)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶

(SEQ ID NO: 27)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 28)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 29)

h²² k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷
k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 30)

k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹

(SEQ ID NO: 31)

k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶

(SEQ ID NO: 32)

k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 33)

k²³ h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 34)

k²³ h²⁴ k²⁵ d²⁶ k²⁷ h²⁸ k²⁹

(SEQ ID NO: 35)

k²³ h²⁴ k²⁵ d²⁶ k²⁷ h²⁸ k²⁹ d³⁰ r³¹ e³² h³³

(SEQ ID NO: 36)

k²³ h²⁴ k²⁵ d²⁶ k²⁷ h²⁸ k²⁹ d³⁰ r³¹ e³² h³³ r³⁴ h³⁵

(SEQ ID NO: 37)

k²³ h²⁴ k²⁵ d²⁶ k²⁷ h²⁸ k²⁹ d³⁰ r³¹ e³² h³³ r³⁴ h³⁵ k³⁶ e³⁷ h³⁸

(SEQ ID NO: 38)

h²⁴ k²⁵ d²⁶ k²⁷ h²⁸ k²⁹ d³⁰ r³¹ e³² h³³ r³⁴ h³⁵ k³⁶

(SEQ ID NO: 39)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹

(SEQ ID NO: 40)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴²

(SEQ ID NO: 41)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴

(SEQ ID NO: 42)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸

(SEQ ID NO: 43)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰

(SEQ ID NO: 44)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷

(SEQ ID NO: 45)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴

(SEQ ID NO: 46)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶

(SEQ ID NO: 47)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 48)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 49)

h²⁴ k²⁵ d²⁶ k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸
s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 50)

k²⁷ h²⁸ k²⁹ d³⁰ r³¹ e³² h³³ r³⁴ h³⁵ k³⁶

(SEQ ID NO: 51)

k²⁷ h²⁸ k²⁹ d³⁰ r³¹ e³² h³³ r³⁴ h³⁵ k³⁶ e³⁷ h³⁸

(SEQ ID NO: 52)

k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹

(SEQ ID NO: 53)

k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶

(SEQ ID NO: 54)

k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 55)

k²⁷ h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 56)

h²⁸ k²⁹ d³⁰ r³¹ e³² h³³ r³⁴ h³⁵ k³⁶

(SEQ ID NO: 57)

h²⁸ k²⁹ d³⁰ r³¹ e³² h³³ r³⁴ h³⁵ k³⁶ e³⁷ h³⁸ k³⁹

(SEQ ID NO: 58)

h²⁸ k²⁹ d³⁰ r³¹ e³² h³³ r³⁴ h³⁵ k³⁶ e³⁷ h³⁸ k³⁹ k⁴⁰ d⁴¹ k⁴²

(SEQ ID NO: 59)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴

(SEQ ID NO: 60)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸

(SEQ ID NO: 61)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰

(SEQ ID NO: 62)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷

(SEQ ID NO: 63)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴

(SEQ ID NO: 64)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶

(SEQ ID NO: 65)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 66)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 67)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 68)

h²⁸ k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰
h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³
g⁷⁴ s⁷⁵ s⁷⁶ d⁷⁷ k⁷⁸

(SEQ ID NO: 69)

k²⁹ d³⁰ r³¹ e³² h³³ r³⁴ h³⁵ k³⁶

(SEQ ID NO: 70)

k²⁹ d³⁰ r³¹ e³² h³³ r³⁴ h³⁵ k³⁶ e³⁷ h³⁸

(SEQ ID NO: 71)

k²⁹ d³⁰ r³¹ e³² h³³ r³⁴ h³⁵ k³⁶ e³⁷ h³⁸ k³⁹

(SEQ ID NO: 72)

k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹

(SEQ ID NO: 73)

k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶

(SEQ ID NO: 74)

k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 75)

k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 76)

k²⁹ d³⁰ r³¹ e³² h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴
s⁷⁵ s⁷⁶ d⁷⁷ k⁷⁸ h⁷⁹

(SEQ ID NO: 77)

h³³ r³⁴ h³⁵ k³⁶ e³⁷ h³⁸ k³⁹ k⁴⁰ d⁴¹ k⁴²

(SEQ ID NO: 78)

h³³ r³⁴ h³⁵ k³⁶ e³⁷ h³⁸ k³⁹ k⁴⁰ d⁴¹ k⁴² d⁴³ k⁴⁴

(SEQ ID NO: 79)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸

(SEQ ID NO: 80)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰

(SEQ ID NO: 81)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷

(SEQ ID NO: 82)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴

(SEQ ID NO: 83)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶

(SEQ ID NO: 84)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 85)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 86)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 87)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸

(SEQ ID NO: 88)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰

(SEQ ID NO: 89)

h³³ r³⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸²

(SEQ ID NO: 90)

h³⁵ k³⁶ e³⁷ h³⁸ k³⁹ k⁴⁰ d⁴¹ k⁴²

(SEQ ID NO: 91)

h³⁵ k³⁶ e³⁷ h³⁸ k³⁹ k⁴⁰ d⁴¹ k⁴² d⁴³ k⁴⁴

(SEQ ID NO: 92)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸

(SEQ ID NO: 93)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰

(SEQ ID NO: 94)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷

(SEQ ID NO: 95)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴
e⁵⁵ h⁵⁶ k⁵⁷ d⁵⁸ s⁵⁹ e⁶⁰ k⁶¹ k⁶² h⁶³ k⁶⁴ e⁶⁵

(SEQ ID NO: 96)

k⁶⁶

(SEQ ID NO: 97)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸

(SEQ ID NO: 98)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 99)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 100)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸

(SEQ ID NO: 101)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰

(SEQ ID NO: 102)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸²

(SEQ ID NO: 103)

h³⁵ k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹
e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴

(SEQ ID NO: 104)

k³⁶ e³⁷ h³⁸ k³⁹ k⁴⁰ d⁴¹ k⁴²

(SEQ ID NO: 105)

k³⁶ e³⁷ h³⁸ k³⁹ k⁴⁰ d⁴¹ k⁴² d⁴³ k⁴⁴

(SEQ ID NO: 106)

k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹

(SEQ ID NO: 107)

k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶

(SEQ ID NO: 108)

k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰
k⁶¹ k⁶² h⁶³

(SEQ ID NO: 109)

k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰
k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 110)

k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰
k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 111)

k³⁶ e³⁷ h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰
k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 112)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶²
h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷
k⁸⁸

(SEQ ID NO: 113)

h³⁸ k³⁹ k⁴⁰ d⁴¹ k⁴² d⁴³ k⁴⁴ d⁴⁵ r⁴⁶ e⁴⁷ k⁴⁸

(SEQ ID NO: 114)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰

(SEQ ID NO: 115)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷

(SEQ ID NO: 116)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴

(SEQ ID NO: 117)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶

(SEQ ID NO: 118)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 119)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 120)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 121)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷
k⁷⁸

(SEQ ID NO: 122)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷
k⁷⁸ h⁷⁹ k⁸⁰

(SEQ ID NO: 123)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷
k⁷⁸ h⁷⁹ k⁸⁰ d⁸¹ k⁸²

(SEQ ID NO: 124)

h³⁸ k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷
d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷
k⁷⁸ h⁷⁹ k⁸⁰ d⁸¹ k⁸² h⁸³ k⁸⁴

(SEQ ID NO: 125)

k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹

(SEQ ID NO: 126)

k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶

(SEQ ID NO: 127)

k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 128)

k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³
k⁶⁴ e⁶⁵ k⁶⁶ e⁶⁷ k⁶⁸ t⁶⁹ k⁷⁰ h⁷¹

(SEQ ID NO: 129)

k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³
k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 130)

k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³
k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 131)

k⁴⁰ d⁴¹ k⁴² d⁴³ k⁴⁴ d⁴⁵ r⁴⁶ e⁴⁷ k⁴⁸ s⁴⁹ k⁵⁰ h⁵¹

(SEQ ID NO: 132)

k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶

(SEQ ID NO: 133)

k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 134)

k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴
e⁶⁵ k⁶⁶ e⁶⁷ k⁶⁸ t⁶⁹ k⁷⁰ h⁷¹

(SEQ ID NO: 135)

k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴
e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 136)

k⁴⁰ d⁴¹ k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴
e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 137)

k⁴² d⁴³ k⁴⁴ d⁴⁵ r⁴⁶ e⁴⁷ k⁴⁸ s⁴⁹ k⁵⁰ h⁵¹

(SEQ ID NO: 138)

k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶

(SEQ ID NO: 139)

k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 140)

k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶
e⁶⁷ k⁶⁸ t⁶⁹ k⁷⁰ h⁷¹

(SEQ ID NO: 141)

k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶
e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 142)

k⁴² d⁴³ k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶
e⁶⁷ k⁶⁸ t⁶⁹ k⁷⁰ h⁷¹ k^{72 d}⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 143)

k⁴⁴ d⁴⁵ r⁴⁶ e⁴⁷ k⁴⁸ s⁴⁹ k⁵⁰ h⁵¹

(SEQ ID NO: 144)

k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶

(SEQ ID NO: 145)

k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 146)

k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸
t⁶⁹ k⁷⁰ h⁷¹

(SEQ ID NO: 147)

k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸
t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 148)

k⁴⁴ d⁴⁵ r⁴⁶ e⁴⁷ k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸
t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 149)

k⁴⁸ s⁴⁹ k⁵⁰ h⁵¹ s⁵² n⁵³ s⁵⁴ e⁵⁵ h⁵⁶ k⁵⁷

(SEQ ID NO: 150)

k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 151)

k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 152)

k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹

(SEQ ID NO: 153)

k⁴⁸ s⁴⁹ k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 154)

k⁵⁰ h⁵¹ s⁵² n⁵³ s⁵⁴ e⁵⁵ h⁵⁶ k⁵⁷

(SEQ ID NO: 155)

k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³

(SEQ ID NO: 156)

k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 157)

k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³
g⁷⁴ s⁷⁵ s⁷⁶ d⁷⁷ k⁷⁸ h⁷⁹

(SEQ ID NO: 158)

k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³
g⁷⁴ s⁷⁵ s⁷⁶ d⁷⁷ k⁷⁸ h⁷⁹ k⁸⁰ d⁸¹ k⁸² h⁸³

(SEQ ID NO: 159)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴

(SEQ ID NO: 160)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶

(SEQ ID NO: 161)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 162)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 163)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 164)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸

(SEQ ID NO: 165)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰

(SEQ ID NO: 166)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸²

(SEQ ID NO: 167)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸² h⁸³ k⁸⁴

(SEQ ID NO: 168)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸

(SEQ ID NO: 169)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰

(SEQ ID NO: 170)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹²

(SEQ ID NO: 171)

h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²
d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴
k⁹⁵

(SEQ ID NO: 172)

h⁵⁶ k⁵⁷ d⁵⁸ s⁵⁹ e⁶⁰ k⁶¹ k⁶² h⁶³ k⁶⁴

(SEQ ID NO: 173)

h⁵⁶ k⁵⁷ d⁵⁸ s⁵⁹ e⁶⁰ k⁶¹ k⁶² h⁶³ k⁶⁴ e⁶⁵ k⁶⁶

(SEQ ID NO: 174)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸

(SEQ ID NO: 175)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰

(SEQ ID NO: 176)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 177)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸

(SEQ ID NO: 178)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰

(SEQ ID NO: 179)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸²

(SEQ ID NO: 180)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴

(SEQ ID NO: 181)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸

(SEQ ID NO: 182)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸ e⁸⁹ k⁹⁰

(SEQ ID NO: 183)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸ e⁸⁹ k⁹⁰ r⁹¹ k⁹²

(SEQ ID NO: 184)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵

(SEQ ID NO: 185)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³

(SEQ ID NO: 186)

h⁵⁶ k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰
d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³ i¹⁰⁴
k¹⁰⁵

(SEQ ID NO: 187)

k⁵⁷ d⁵⁸ s⁵⁹ e⁶⁰ k⁶¹ k⁶² h⁶³ k⁶⁴

(SEQ ID NO: 188)

k⁵⁷ d⁵⁸ s⁵⁹ e⁶⁰ k⁶¹ k⁶² h⁶³ k⁶⁴ e⁶⁵ k⁶⁶

(SEQ ID NO: 189)

k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 190)

k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 191)

k⁵⁷ d⁵⁸ s⁵⁹ e⁶⁰ k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹
k⁸² h⁸³

(SEQ ID NO: 192)

k⁶¹ k⁶² h⁶³ k⁶⁴ e⁶⁵ k⁶⁶ e⁶⁷ k⁶⁸

(SEQ ID NO: 193)

k⁶¹ k⁶² h⁶³ k⁶⁴ e⁶⁵ k⁶⁶ e⁶⁷ k⁶⁸ t⁶⁹ k⁷⁰ h⁷¹

(SEQ ID NO: 194)

k⁶¹ k⁶² h⁶³ k⁶⁴ e⁶⁵ k⁶⁶ e⁶⁷ k⁶⁸ t⁶⁹ k⁷⁰

(SEQ ID NO: 195)

k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 196)

k⁶¹ k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 197)

k⁶² h⁶³ k⁶⁴ e⁶⁵ k⁶⁶ e⁶⁷ k⁶⁸

(SEQ ID NO: 198)

k⁶² h⁶³ k⁶⁴ e⁶⁵ k⁶⁶ e⁶⁷ k⁶⁸ t⁶⁹ k⁷⁰ h⁷¹

(SEQ ID NO: 199)

k⁶² h⁶³ k⁶⁴ e⁶⁵ k⁶⁶ e⁶⁷ k⁶⁸ t⁶⁹ k⁷⁰

(SEQ ID NO: 200)

k⁶² h⁶³ k⁶⁴ e⁶⁵ k⁶⁶ e⁶⁷ k⁶⁸ t⁶⁹ k⁷⁰ h⁷¹ k⁷²

(SEQ ID NO: 201)

k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 202)

k⁶² h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 203)

h⁶³ k⁶⁴ e⁶⁵ k⁶⁶ e⁶⁷ k⁶⁸ t⁶⁹ k⁷⁰

(SEQ ID NO: 204)

h⁶³ k⁶⁴ e⁶⁵ k⁶⁶ e⁶⁷ k⁶⁸ t⁶⁹ k⁷⁰ h⁷¹ k⁷²

(SEQ ID NO: 205)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵
r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³

(SEQ ID NO: 206)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵
r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³ i¹⁰⁴ k¹⁰⁵

(SEQ ID NO: 207)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸

(SEQ ID NO: 208)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰

(SEQ ID NO: 209)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸²

(SEQ ID NO: 210)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴

(SEQ ID NO: 211)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸

(SEQ ID NO: 212)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸
e⁸⁹ k⁹⁰

(SEQ ID NO: 213)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸
e⁸⁹ k⁹⁰ r⁹¹ k⁹²

(SEQ ID NO: 214)

h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸
e⁸⁹ k⁹⁰ r⁹¹ k⁹² e⁹³ e⁹⁴ k⁹⁵

(SEQ ID NO: 215)

k⁶⁴ e⁶⁵ k⁶⁶ e⁶⁷ k⁶⁸ t⁶⁹ k⁷⁰ h⁷¹

(SEQ ID NO: 216)

k⁶⁴ e⁶⁵ k⁶⁶ e⁶⁷ k⁶⁸ t⁶⁹ k⁷⁰ h⁷¹ k⁷²

(SEQ ID NO: 217)

k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 218)

k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 219)

k⁶⁶ e⁶⁷ k⁶⁸ t⁶⁹ k⁷⁰ h⁷¹ k⁷²

(SEQ ID NO: 220)

k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 221)

k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 222)

k⁶⁸ t⁶⁹ k⁷⁰ h⁷¹ k⁷² d⁷³ g⁷⁴ s⁷⁵ s⁷⁶ d⁷⁷ k⁷⁸

(SEQ ID NO: 223)

k⁶⁸ t⁶⁹ k⁷⁰ h⁷¹ k⁷² d⁷³ g⁷⁴ s⁷⁵ s⁷⁶ d⁷⁷ k⁷⁸ h⁷⁹

(SEQ ID NO: 224)

k⁶⁸ t⁶⁹ k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 225)

k⁷⁰ h⁷¹ k⁷² d⁷³ g⁷⁴ s⁷⁵ s⁷⁶ d⁷⁷ k⁷⁸

(SEQ ID NO: 226)

k⁷⁰ h⁷¹ k⁷² d⁷³ g⁷⁴ s⁷⁵ s⁷⁶ d⁷⁷ k⁷⁸ h⁷⁹

(SEQ ID NO: 227)

k⁷⁰ h⁷¹ k⁷² d⁷³ g⁷⁴ s⁷⁵ s⁷⁶ d⁷⁷ k⁷⁸ h⁷⁹ k⁸⁰

(SEQ ID NO: 228)

k⁷⁰ h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 229)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶
r⁹⁷ a⁹⁸ a⁹⁹ g¹⁰⁰ d¹⁰¹ a¹⁰² k¹⁰³

(SEQ ID NO: 230)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶
r⁹⁷ a⁹⁸ a⁹⁹ g¹⁰⁰ d¹⁰¹ a¹⁰² k¹⁰³ i¹⁰⁴ k¹⁰⁵

(SEQ ID NO: 231)

h⁷¹ k⁷² d⁷³ g⁷⁴ s⁷⁵ s⁷⁶ d⁷⁷ k⁷⁸

(SEQ ID NO: 232)

h⁷¹ k⁷² d⁷³ g⁷⁴ s⁷⁵ s⁷⁶ d⁷⁷ k⁷⁸ h⁷⁹ k⁸⁰

(SEQ ID NO: 233)

h⁷¹ k⁷² d⁷³ g⁷⁴ s⁷⁵ s⁷⁶ d⁷⁷ k⁷⁸ h⁷⁹ k⁸⁰ d⁸¹ k⁸²

(SEQ ID NO: 234)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴

(SEQ ID NO: 235)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸

(SEQ ID NO: 236)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰

(SEQ ID NO: 237)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹²

(SEQ ID NO: 238)

h⁷¹ k⁷² d⁷³ g⁷⁴ s⁷⁵ s⁷⁶ d⁷⁷ k⁷⁸ h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵

(SEQ ID NO: 239)

k⁷² d⁷³ g⁷⁴ s⁷⁵ s⁷⁶ d⁷⁷ k⁷⁸ h⁷⁹

(SEQ ID NO: 240)

k⁷² d⁷³ g⁷⁴ s⁷⁵ s⁷⁶ d⁷⁷ k⁷⁸ h⁷⁹ k⁸⁰

(SEQ ID NO: 241)

k⁷² d⁷³ g⁷⁴ s⁷⁵ s⁷⁶ d⁷⁷ k⁷⁸ h⁷⁹ k⁸⁰ d⁸¹ k⁸²

(SEQ ID NO: 242)

k⁷² d⁷³ g⁷⁴ s⁷⁵ s⁷⁶ d⁷⁷ k⁷⁸ h⁷⁹ k⁸⁰ d⁸¹ k⁸² h⁸³

(SEQ ID NO: 243)

k⁷⁸ h⁷⁹ k⁸⁰ d⁸¹ k⁸² h⁸³ k⁸⁴

(SEQ ID NO: 244)

k⁷⁸ h⁷⁹ k⁸⁰ d⁸¹ k⁸² h⁸³ k⁸⁴ d⁸⁵ r⁸⁶ d⁸⁷ k⁸⁸

(SEQ ID NO: 245)

h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³

(SEQ ID NO: 246)

h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³
i¹⁰⁴ k¹⁰⁵

(SEQ ID NO: 247)

h⁷⁹ k⁸⁰ d⁸¹ k⁸² h⁸³ k⁸⁴ d⁸⁵ r⁸⁶ d⁸⁷ k⁸⁸

(SEQ ID NO: 248)

h⁷⁹ k⁸⁰ d⁸¹ k⁸² h⁸³ k⁸⁴ d⁸⁵ r⁸⁶ d⁸⁷ k⁸⁸ e⁸⁹ k⁹⁰

(SEQ ID NO: 249)

h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹²

(SEQ ID NO: 250)

h⁷⁹ k⁸⁰ d⁸¹ k⁸² h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵

(SEQ ID NO: 251)

k⁸⁰ d⁸¹ k⁸² h⁸³ k⁸⁴ d⁸⁵ r⁸⁶ d⁸⁷ k⁸⁸

(SEQ ID NO: 252)

k⁸⁰ d⁸¹ k⁸² h⁸³ k⁸⁴ d⁸⁵ r⁸⁶ d⁸⁷ k⁸⁸ e⁸⁹ k⁹⁰

(SEQ ID NO: 253)

k⁸² h⁸³ k⁸⁴ d⁸⁵ r⁸⁶ d⁸⁷ k⁸⁸

(SEQ ID NO: 254)

k⁸² h⁸³ k⁸⁴ d⁸⁵ r⁸⁶ d⁸⁷ k⁸⁸ e⁸⁹ k⁹⁰

(SEQ ID NO: 255)

k⁸² h⁸³ k⁸⁴ d⁸⁵ r⁸⁶ d⁸⁷ k⁸⁸ e⁸⁹ k⁹⁰ r⁹¹ k⁹²

(SEQ ID NO: 256)

h⁸³ k⁸⁴ d⁸⁵ r⁸⁶ d⁸⁷ k⁸⁸ e⁸⁹ k⁹⁰

(SEQ ID NO: 257)

h⁸³ k⁸⁴ d⁸⁵ r⁸⁶ d⁸⁷ k⁸⁸ e⁸⁹ k⁹⁰ r⁹¹ k⁹²

(SEQ ID NO: 258)

h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³

(SEQ ID NO: 259)

h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵ i⁹⁶ r⁹⁷ a⁹⁸ a⁹⁹ g¹⁰⁰ d¹⁰¹ a¹⁰² k¹⁰³ i¹⁰⁴ k¹⁰⁵

(SEQ ID NO: 260)

h⁸³ k⁸⁴ d⁸⁵ r⁸⁶ d⁸⁷ k⁸⁸ e⁸⁹ k⁹⁰ r⁹¹ k⁹² e⁹³ e⁹⁴ k⁹⁵

(SEQ ID NO: 261)

k¹⁷⁴ l¹⁷⁵ k¹⁷⁶ k¹⁷⁷ p¹⁷⁸ k¹⁷⁹ n¹⁸⁰ k¹⁸¹ d¹⁸² k¹⁸³ d¹⁸⁴ k¹⁸⁵ k¹⁸⁶ v¹⁸⁷ a¹⁸⁸ e¹⁸⁹ p¹⁹⁰ d¹⁹¹ n¹⁹² k¹⁹³
k¹⁹⁴ k¹⁹⁵ k¹⁹⁶ a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³
p²¹⁴ e²¹⁵ g²¹⁶ i²¹⁷ k²¹⁸ w²¹⁹ k²²⁰ f²²¹ l²²² e²²³ h²²⁴

(SEQ ID NO: 262)

k¹⁷⁶ k¹⁷⁷ p¹⁷⁸ k¹⁷⁹ n¹⁸⁰ k¹⁸¹ d¹⁸² k¹⁸³ d¹⁸⁴ k¹⁸⁵ k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵
k¹⁹⁶ a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵
g²¹⁶ i²¹⁷ k²¹⁸ w²¹⁹ k²²⁰ f²²¹ l²²² e²²³ h²²⁴

(SEQ ID NO: 263)

k¹⁷⁷ p¹⁷⁸ k¹⁷⁹ n¹⁸⁰ k¹⁸¹ d¹⁸² k¹⁸³ d¹⁸⁴ k¹⁸⁵ k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵ k¹⁹⁶
a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶
i²¹⁷ k²¹⁸ w²¹⁹ k²²⁰ f²²¹ l²²² e²²³ h²²⁴

(SEQ ID NO: 264)

k¹⁷⁹ n¹⁸⁰ k¹⁸¹ d¹⁸² k¹⁸³ d¹⁸⁴ k¹⁸⁵ k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵ k¹⁹⁶ a¹⁹⁷ k¹⁹⁸
k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷ k²¹⁸
w²¹⁹ k²²⁰ f²²¹ l²²² e²²³ h²²⁴

(SEQ ID NO: 265)

k¹⁸³ d¹⁸⁴ k¹⁸⁵ k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵ k¹⁹⁶ a¹⁹⁷ k¹⁹⁸ k¹⁹⁹ e²⁰⁰ e²⁰¹ e²⁰²
q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷ k²¹⁸ w²¹⁹ k²²⁰ f²²¹ l²²²
e²²³ h²²⁴

(SEQ ID NO: 266)

k¹⁸⁵ k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵ k¹⁹⁶ a¹⁹⁷ k¹⁹⁸ k¹⁹⁹ e²⁰⁰ e²⁰¹ e²⁰² q²⁰³ k²⁰⁴
w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷ k²¹⁸ w²¹⁹ k²²⁰ f²²¹ l²²² e²²³ h²²⁴

(SEQ ID NO: 267)

k¹⁸⁶ v¹⁸⁷ a¹⁸⁸ e¹⁸⁹ p¹⁹⁰ d¹⁹¹ n¹⁹² k¹⁹³ k¹⁹⁴ k¹⁹⁵ k¹⁹⁶ a¹⁹⁷ k¹⁹⁸ k¹⁹⁹ e²⁰⁰ e²⁰¹ e²⁰² q²⁰³ k²⁰⁴ w²⁰⁵
k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷ k²¹⁸ w²¹⁹ k²²⁰ f²²¹ l²²² e²²³ h²²⁴

(SEQ ID NO: 268)

k¹⁹³ k¹⁹⁴ k¹⁹⁵ k¹⁹⁶ a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹²
y²¹³ p²¹⁴ e²¹⁵ g²¹⁶ i²¹⁷ k²¹⁸ w²¹⁹ k²²⁰ f²²¹ l²²² e²²³ h²²⁴

(SEQ ID NO: 269)

k¹⁹⁴ k¹⁹⁵ k¹⁹⁶ a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³
p²¹⁴ e²¹⁵ g²¹⁶ i²¹⁷ k²¹⁸ w²¹⁹ k²²⁰ f²²¹ l²²² e²²³ h²²⁴

(SEQ ID NO: 270)

k¹⁹⁵ k¹⁹⁶ a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴
e²¹⁵ g²¹⁶ i²¹⁷ k²¹⁸ w²¹⁹ k²²⁰ f²²¹ l²²² e²²³ h²²⁴

(SEQ ID NO: 271)

k¹⁹⁶ a¹⁹⁷ k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵
g²¹⁶ i²¹⁷ k²¹⁸ w²¹⁹ k²²⁰ f²²¹ l²²² e²²³ h²²⁴

(SEQ ID NO: 272)

k¹⁹⁸ k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷
k²¹⁸ w²¹⁹ k²²⁰ f²²¹ l²²² e²²³ h²²⁴

(SEQ ID NO: 273)

k¹⁹⁹ e²⁰⁰ e²⁰¹ e²⁰² q²⁰³ k²⁰⁴ w²⁰⁵ k²⁰⁶ w²⁰⁷ w²⁰⁸ e²⁰⁹ e²¹⁰ e²¹¹ r²¹² y²¹³ p²¹⁴ e²¹⁵ g²¹⁶ i²¹⁷ k²¹⁸
w²¹⁹ k²²⁰ f²²¹ l²²² e²²³ h²²⁴

(SEQ ID NO: 274)

k²¹⁸ w²¹⁹ k²²⁰ f²²¹ l²²² e²²³ h²²⁴ k²²⁵ g²²⁶ p²²⁷ v²²⁸ f²²⁹ a²³⁰ p²³¹ p²³² y²³³ e²³⁴ p²³⁵ l²³⁶ p²³⁷
e²³⁸ g²³⁹ v²⁴⁰ k²⁴¹ f²⁴² y²⁴³ y²⁴⁴ d²⁴⁵ g²⁴⁶ k²⁴⁷ v²⁴⁸ m²⁴⁹ k²⁵⁰ l²⁵¹ s²⁵² p²⁵³ k²⁵⁴ a²⁵⁵ e²⁵⁶ e²⁵⁷
v²⁵⁸ a²⁵⁹ t²⁶⁰ f²⁶¹ f²⁶² a²⁶³ k²⁶⁴ m²⁶⁵ l²⁶⁶ d²⁶⁷ h²⁶⁸

(SEQ ID NO: 275)

k²¹⁸ w²¹⁹ k²²⁰ f²²¹ l²²² e²²³ h²²⁴ k²²⁵

(SEQ ID NO: 276)

h²²⁴ k²²⁵ g²²⁶ p²²⁷ v²²⁸ f²²⁹ a²³⁰ p²³¹ p²³² y²³³ e²³⁴ p²³⁵ l²³⁶ p²³⁷ e²³⁸ g²³⁹ v²⁴⁰ k²⁴¹ f²⁴² y²⁴³
y²⁴⁴ d²⁴⁵ g²⁴⁶ k²⁴⁷

(SEQ ID NO: 277)

h²²⁴ k²²⁵ g²²⁶ p²²⁷ v²²⁸ f²²⁹ a²³⁰ p²³¹ p²³² y²³³ e²³⁴ p²³⁵ l²³⁶ p²³⁷ e²³⁸ g²³⁹ v²⁴⁰ k²⁴¹ f²⁴² y²⁴³
y²⁴⁴ d²⁴⁵ g²⁴⁶ k²⁴⁷ v²⁴⁸ m²⁴⁹ k²⁵⁰

(SEQ ID NO: 278)

h²²⁴ k²²⁵ g²²⁶ p²²⁷ v²²⁸ f²²⁹ a²³⁰ p²³¹ p²³² y²³³ e²³⁴ p²³⁵ l²³⁶ p²³⁷ e²³⁸ g²³⁹ v²⁴⁰ k²⁴¹ f²⁴² y²⁴³
y²⁴⁴ d²⁴⁵ g²⁴⁶ k²⁴⁷ v²⁴⁸ m²⁴⁹ k²⁵⁰ l²⁵¹ s²⁵² p²⁵³ k²⁵⁴

(SEQ ID NO: 279)

h²²⁴ k²²⁵ g²²⁶ p²²⁷ v²²⁸ f²²⁹ a²³⁰ p²³¹ p²³² y²³³ e²³⁴ p²³⁵ l²³⁶ p²³⁷ e²³⁸ g²³⁹ v²⁴⁰ k²⁴¹ f²⁴² y²⁴³
y²⁴⁴ d²⁴⁵ g²⁴⁶ k²⁴⁷ v²⁴⁸ m²⁴⁹ k²⁵⁰ l²⁵¹ s²⁵² p²⁵³ k²⁵⁴ a²⁵⁵ e²⁵⁶ e²⁵⁷ v²⁵⁸ a²⁵⁹ t²⁶⁰ f²⁶¹ f²⁶² a²⁶³
k²⁶⁴

(SEQ ID NO: 280)

h²²⁴ k²²⁵ g²²⁶ p²²⁷ v²²⁸ f²²⁹ a²³⁰ p²³¹ p²³² y²³³ e²³⁴ p²³⁵ l²³⁶ p²³⁷ e²³⁸ g²³⁹ v²⁴⁰ k²⁴¹ f²⁴² y²⁴³
y²⁴⁴ d²⁴⁵ g²⁴⁶ k²⁴⁷ v²⁴⁸ m²⁴⁹ k²⁵⁰ l²⁵¹ s²⁵² p²⁵³ k²⁵⁴ a²⁵⁵ e²⁵⁶ e²⁵⁷ v²⁵⁸ a²⁵⁹ t²⁶⁰ f²⁶¹ f²⁶² a²⁶³
k²⁶⁴ m²⁶⁵ l²⁶⁶ d²⁶⁷ h²⁶⁸ e²⁶⁹ y²⁷⁰ t²⁷¹ t²⁷² k²⁷³

(SEQ ID NO: 281)

k²⁴¹ f²⁴² y²⁴³ y²⁴⁴ d²⁴⁵ g²⁴⁶ k²⁴⁷ v²⁴⁸ m²⁴⁹ k²⁵⁰ l²⁵¹ s²⁵² p²⁵³ k²⁵⁴ a²⁵⁵ e²⁵⁶ e²⁵⁷ v²⁵⁸ a²⁵⁹ t²⁶⁰
f²⁶¹ f²⁶² a²⁶³ k²⁶⁴ m²⁶⁵ l²⁶⁶ d²⁶⁷ h²⁶⁸

(SEQ ID NO: 282)

k²⁴⁷ v²⁴⁸ m²⁴⁹ k²⁵⁰ l²⁵¹ s²⁵² p²⁵³ k²⁵⁴ a²⁵⁵ e²⁵⁶ e²⁵⁷ v²⁵⁸ a²⁵⁹ t²⁶⁰ f²⁶¹ f²⁶² a²⁶³ k²⁶⁴ m²⁶⁵ l²⁶⁶
d²⁶⁷ h²⁶⁸

(SEQ ID NO: 283)

k²⁵⁴ a²⁵⁵ e²⁵⁶ e²⁵⁷ v²⁵⁸ a²⁵⁹ t²⁶⁰ f²⁶¹ f²⁶² a²⁶³ k²⁶⁴ m²⁶⁵ l²⁶⁶ d²⁶⁷ h²⁶⁸

The following Replikin sequences were identified in the mid-molecule of Accession Number Q9WUL0:

(SEQ ID NO: 284)
k²⁶⁴ m²⁶⁵ l²⁶⁶ d²⁶⁷ h²⁶⁸ e²⁶⁹ y²⁷⁰ t²⁷¹ t²⁷² k²⁷³
(SEQ ID NO: 285)
h²⁶⁸ e²⁶⁹ y²⁷⁰ t²⁷¹ t²⁷² k²⁷³ e²⁷⁴ i²⁷⁵ f²⁷⁶ r²⁷⁷ k²⁷⁸ n²⁷⁹ f²⁸⁰ f²⁸¹ k²⁸²
(SEQ ID NO: 286)
h²⁶⁸ e²⁶⁹ y²⁷⁰ t²⁷¹ t²⁷² k²⁷³ e²⁷⁴ i²⁷⁵ f²⁷⁶ r²⁷⁷ k²⁷⁸ n²⁷⁹ f²⁸⁰ f²⁸¹ k²⁸² d²⁸³ w²⁸⁴ r²⁸⁵ k²⁸⁶
(SEQ ID NO: 287)
h²⁶⁸ e²⁶⁹ y²⁷⁰ t²⁷¹ t²⁷² k²⁷³ e²⁷⁴ i²⁷⁵ f²⁷⁶ r²⁷⁷ k²⁷⁸ n²⁷⁹ f²⁸⁰ f²⁸¹ k²⁸² d²⁸³ w²⁸⁴ r²⁸⁵ k²⁸⁶
e²⁸⁷ m²⁸⁸ t²⁸⁹ n²⁹⁰ d²⁹¹ e²⁹² k²⁹³ n²⁹⁴ t²⁹⁵ i²⁹⁶ t²⁹⁷ n²⁹⁸ l²⁹⁹ s³⁰⁰ k³⁰¹
(SEQ ID NO: 288)
h²⁶⁸ e²⁶⁹ y²⁷⁰ t²⁷¹ t²⁷² k²⁷³ e²⁷⁴ i²⁷⁵ f²⁷⁶ r²⁷⁷ k²⁷⁸ n²⁷⁹ f²⁸⁰ f²⁸¹ k²⁸² d²⁸³ w²⁸⁴ r²⁸⁵ k²⁸⁶
e²⁸⁷ m²⁸⁸ t²⁸⁹ n²⁹⁰ d²⁹¹ e²⁹² k²⁹³
(SEQ ID NO: 289)
k³¹² a³¹³ q³¹⁴ s³¹⁵ e³¹⁶ a³¹⁷ r³¹⁸ k³¹⁹ q³²⁰ m³²¹ s³²² k³²³ e³²⁴ e³²⁵ k³²⁶ l³²⁷ k³²⁸ i³²⁹ k³³⁰
e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴ m³⁴⁵ d³⁴⁶ n³⁴⁷ h³⁴⁸
(SEQ ID NO: 290)
k³¹⁹ q³²⁰ m³²¹ s³²² k³²³ e³²⁴ e³²⁵ k³²⁶ l³²⁷ k³²⁸ i³²⁹ k³³⁰ e³³¹ e³³² n³³³ e³³⁴ k³³⁵ l³³⁶ l³³⁷
k³³⁸ e³³⁹ y³⁴⁰ g³⁴¹ f³⁴² c³⁴³ v³⁴⁴ m³⁴⁵ d³⁴⁶ n³⁴⁷ h³⁴⁸
(SEQ ID NO: 291)
k³¹⁹ q³²⁰ m³²¹ s³²² k³²³ e³²⁴ e³²⁵ k³²⁶ l³²⁷ k³²⁸ i³²⁹ k³³⁰ e³³¹ e³³² n³³³ e³³⁴ k³³⁵ l³³⁶ l³³⁷
³³⁸ e³³⁹ y³⁴⁰ g³⁴¹ f³⁴² c³⁴³ v³⁴⁴ m³⁴⁵ d³⁴⁶ n³⁴⁷ h³⁴⁸ r³⁴⁹ e³⁵⁰ r³⁵¹ i³⁵² a³⁵³ n³⁵⁴ f³⁵⁵ k³⁵⁶
i³⁵⁷ e³⁵⁸ p³⁵⁹ p³⁶⁰ g³⁶¹ l³⁶² f³⁶³ r³⁶⁴ g³⁶⁵ r³⁶⁶ g³⁶⁷ n³⁶⁸ h³⁶⁹
(SEQ ID NO: 292)
k³²³ e³²⁴ e³²⁵ k³²⁶ l³²⁷ k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹
f³⁴² c³⁴³ v³⁴⁴ m³⁴⁵ d³⁴⁶ n³⁴⁷ h³⁴⁸
(SEQ ID NO: 293)
k³²³ e³²⁴ e³²⁵ k³²⁶ l³²⁷ k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹
f³⁴² c³⁴³ v³⁴⁴ m³⁴⁵ d³⁴⁶ n³⁴⁷ h³⁴⁸ r³⁴⁹ e³⁵⁰ r³⁵¹ i³⁵² a³⁵³ n³⁵⁴ f³⁵⁵ k³⁵⁶ i³⁵⁷ e³⁵⁸ p³⁵⁹ p³⁶⁰
g³⁶¹ l³⁶² f³⁶³ r³⁶⁴ g³⁶⁵ r³⁶⁶ g³⁶⁷ n³⁶⁸ h³⁶⁹
(SEQ ID NO: 294)
k³²⁶ l³²⁷ k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴
m³⁴⁵ d³⁴⁶ n³⁴⁷ h³⁴⁸
(SEQ ID NO: 295)
k³²⁶ l³²⁷ k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴
m³⁴⁵ d³⁴⁶ n³⁴⁷ h³⁴⁸ r³⁴⁹ e³⁵⁰ r³⁵¹ i³⁵² a³⁵³ n³⁵⁴ f³⁵⁵ k³⁵⁶ i³⁵⁷ e³⁵⁸ p³⁵⁹ p³⁶⁰ g³⁶¹ l³⁶² f³⁶³
r³⁶⁴ g³⁶⁵ r³⁶⁶ g³⁶⁷ n³⁶⁸ h³⁶⁹
(SEQ ID NO: 296)
k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴ m³⁴⁵ d³⁴⁶
n³⁴⁷ h³⁴⁸
(SEQ ID NO: 297)
k³²⁸ i³²⁹ k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴ m³⁴⁵ d³⁴⁶
n³⁴⁷ h³⁴⁸ r³⁴⁹ e³⁵⁰ r³⁵¹ i³⁵² a³⁵³ n³⁵⁴ f³⁵⁵ k³⁵⁶ i³⁵⁷ e³⁵⁸ p³⁵⁹ p³⁶⁰ g³⁶¹ l³⁶² f³⁶³ r³⁶⁴ g³⁶⁵
r³⁶⁶ g³⁶⁷ n³⁶⁸ h³⁶⁹
(SEQ ID NO: 298)
k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴ m³⁴⁵ d³⁴⁶ n³⁴⁷ h³⁴⁸
(SEQ ID NO: 299)
k³³⁰ e³³¹ e³³² n³³³ e³³⁴ k³³⁵ l³³⁶ l³³⁷ k³³⁸ e³³⁹ y³⁴⁰ g³⁴¹ f³⁴² c³⁴³ v³⁴⁴ m³⁴⁵ d³⁴⁶ n³⁴⁷ h³⁴⁸
r³⁴⁹ e³⁵⁰ r³⁵¹ i³⁵² a³⁵³ n³⁵⁴ f³⁵⁵ k³⁵⁶ i³⁵⁷ e³⁵⁸ p³⁵⁹ p³⁶⁰ g³⁶¹ l³⁶² f³⁶³ r³⁶⁴ g³⁶⁵ r³⁶⁶ g³⁶⁷
n³⁶⁸ h³⁶⁹
(SEQ ID NO: 300)
h³⁶⁹ p³⁷⁰ k³⁷¹ m³⁷² g³⁷³ m³⁷⁴ l³⁷⁵ k³⁷⁶ r³⁷⁷ r³⁷⁸ i³⁷⁹ m³⁸⁰ p³⁸¹ e³⁸² d³⁸³ i³⁸⁴ i³⁸⁵ i³⁸⁶ n³⁸⁷
c³⁸⁸ s³⁸⁹ k³⁹⁰ d³⁹¹ a³⁹² k³⁹³ v³⁹⁴ p³⁹⁵ s³⁹⁶ p³⁹⁷ p³⁹⁸ p³⁹⁹ g⁴⁰⁰ h⁴⁰¹ k⁴⁰²
(SEQ ID NO: 301)
h³⁶⁹ p³⁷⁰ k³⁷¹ m³⁷² g³⁷³ m³⁷⁴ l³⁷⁵ k³⁷⁶ r³⁷⁷ r³⁷⁸ i³⁷⁹ m³⁸⁰ p³⁸¹ e³⁸² d³⁸³ i³⁸⁴ i³⁸⁵ i³⁸⁶ n³⁸⁷
c³⁸⁸ s³⁸⁹ k³⁹⁰ d³⁹¹ a³⁹² k³⁹³ v³⁹⁴ p³⁹⁵ s³⁹⁶ p³⁹⁷ p³⁹⁸ p³⁹⁹ g⁴⁰⁰ h⁴⁰¹ k⁴⁰² w⁴⁰³ k⁴⁰⁴ e⁴⁰⁵ v⁴⁰⁶
r⁴⁰⁷ h⁴⁰⁸ d⁴⁰⁹ n⁴¹⁰ k⁴¹¹
(SEQ ID NO: 302)
k³⁹³ v³⁹⁴ p³⁹⁵ s³⁹⁶ p³⁹⁷ p³⁹⁸ p³⁹⁹ g⁴⁰⁰ h⁴⁰¹ k⁴⁰²
(SEQ ID NO: 303)
k³⁹³ v³⁹⁴ p³⁹⁵ s³⁹⁶ p³⁹⁷ p³⁹⁸ p³⁹⁹ g⁴⁰⁰ h⁴⁰¹ k⁴⁰² w⁴⁰³ k⁴⁰⁴ e⁴⁰⁵ v⁴⁰⁶ r⁴⁰⁷ h⁴⁰⁸
(SEQ ID NO: 304)
h⁴⁰¹ k⁴⁰² w⁴⁰³ k⁴⁰⁴ e⁴⁰⁵ v⁴⁰⁶ r⁴⁰⁷ h⁴⁰⁸ d⁴⁰⁹ n⁴¹⁰ k⁴¹¹
(SEQ ID NO: 305)
h⁴⁰¹ k⁴⁰² w⁴⁰³ k⁴⁰⁴ e⁴⁰⁵ v⁴⁰⁶ r⁴⁰⁷ h⁴⁰⁸ d⁴⁰⁹ n⁴¹⁰ k⁴¹¹ v⁴¹² t⁴¹³ w⁴¹⁴ l⁴¹⁵ v⁴¹⁶ s⁴¹⁷ w⁴¹⁸ t⁴¹⁹
e⁴²⁰ n⁴²¹ i⁴²² q⁴²³ g⁴²⁴ s⁴²⁵ i⁴²⁶ k⁴²⁷ y⁴²⁸ i⁴²⁹ m⁴³⁰ l⁴³¹ n⁴³² p⁴³³ s⁴³⁴ s⁴³⁵ r⁴³⁶ i⁴³⁷ k⁴³⁸
g⁴³⁹ e⁴⁴⁰ k⁴⁴¹ d⁴⁴² w⁴⁴³ q⁴⁴⁴ k⁴⁴⁵
(SEQ ID NO: 306)
k⁴⁰² w⁴⁰³ k⁴⁰⁴ e⁴⁰⁵ v⁴⁰⁶ r⁴⁰⁷ h⁴⁰⁸ d⁴⁰⁹ n⁴¹⁰ k⁴¹¹
(SEQ ID NO: 307)
k⁴⁰⁴ e⁴⁰⁵ v⁴⁰⁶ r⁴⁰⁷ h⁴⁰⁸ d⁴⁰⁹ n⁴¹⁰ k⁴¹¹
(SEQ ID NO: 308)
h⁴⁰⁸ d⁴⁰⁹ n⁴¹⁰ k⁴¹¹ v⁴¹² t⁴¹³ w⁴¹⁴ l⁴¹⁵ v⁴¹⁶ s⁴¹⁷ w⁴¹⁸ t⁴¹⁹ e⁴²⁰ n⁴²¹ i⁴²² q⁴²³ g⁴²⁴ s⁴²⁵ i⁴²⁶
k⁴²⁷ y⁴²⁸ i⁴²⁹ m⁴³⁰ l⁴³¹ n⁴³² p⁴³³ s⁴³⁴ s⁴³⁵ r⁴³⁶ i⁴³⁷ k⁴³⁸ g⁴³⁹ e⁴⁴⁰ k⁴⁴¹ d⁴⁴² w⁴⁴³ q⁴⁴⁴ k⁴⁴⁵
(SEQ ID NO: 309)
h⁴⁰⁸ d⁴⁰⁹ n⁴¹⁰ k⁴¹¹ v⁴¹² t⁴¹³ w⁴¹⁴ l⁴¹⁵ v⁴¹⁶ s⁴¹⁷ w⁴¹⁸ t⁴¹⁹ e⁴²⁰ n⁴²¹ i⁴²² q⁴²³ g⁴²⁴ s⁴²⁵ i⁴²⁶
k⁴²⁷ y⁴²⁸ i⁴²⁹ m⁴³⁰ l⁴³¹ n⁴³² p⁴³³ s⁴³⁴ s⁴³⁵ r⁴³⁶ i⁴³⁷ k⁴³⁸ g⁴³⁹ e⁴⁴⁰ k⁴⁴¹ d⁴⁴² w⁴⁴³ q⁴⁴⁴ k⁴⁴⁵
y⁴⁴⁶ e⁴⁴⁷ t⁴⁴⁸ a⁴⁴⁹ r⁴⁵⁰ r⁴⁵¹ l⁴⁵² k⁴⁵³
(SEQ ID NO: 310)
h⁴⁰⁸ d⁴⁰⁹ n⁴¹⁰ k⁴¹¹ v⁴¹² t⁴¹³ w⁴¹⁴ l⁴¹⁵ v⁴¹⁶ s⁴¹⁷ w⁴¹⁸ t⁴¹⁹ e⁴²⁰ n⁴²¹ i⁴²² q⁴²³ g⁴²⁴ s⁴²⁵ i⁴²⁶
k⁴²⁷ y⁴²⁸ i⁴²⁹ m⁴³⁰ l⁴³¹ n⁴³² p⁴³³ s⁴³⁴ s⁴³⁵ r⁴³⁶ i⁴³⁷ k⁴³⁸ g⁴³⁹ e⁴⁴⁰ k⁴⁴¹ d⁴⁴² w⁴⁴³ q⁴⁴⁴ k⁴⁴⁵
y⁴⁴⁶ e⁴⁴⁷ t⁴⁴⁸ a⁴⁴⁹ r⁴⁵⁰ r⁴⁵¹ l⁴⁵² k⁴⁵³ k⁴⁵⁴
(SEQ ID NO: 311)
k⁴⁸⁶ l⁴⁸⁷ a⁴⁸⁸ l⁴⁸⁹ r⁴⁹⁰ a⁴⁹¹ g⁴⁹² n⁴⁹³ e⁴⁹⁴ k⁴⁹⁵ e⁴⁹⁶ e⁴⁹⁷ g⁴⁹⁸ e⁴⁹⁹ t⁵⁰⁰ a⁵⁰¹ d⁵⁰² t⁵⁰³ v⁵⁰⁴
g⁵⁰⁵ c⁵⁰⁶ c⁵⁰⁷ s⁵⁰⁸ l⁵⁰⁹ r⁵¹⁰ v⁵¹¹ e⁵¹² h⁵¹³
(SEQ ID NO: 312)
k⁴⁸⁶ l⁴⁸⁷ a⁴⁸⁸ l⁴⁸⁹ r⁴⁹⁰ a⁴⁹¹ g⁴⁹² n⁴⁹³ e⁴⁹⁴ k⁴⁹⁵ e⁴⁹⁶ e⁴⁹⁷ g⁴⁹⁸ e⁴⁹⁹ t⁵⁰⁰ a⁵⁰¹ d⁵⁰² t⁵⁰³ v⁵⁰⁴
g⁵⁰⁵ c⁵⁰⁶ c⁵⁰⁷ s⁵⁰⁸ l⁵⁰⁹ r⁵¹⁰ v⁵¹¹ e⁵¹² h⁵¹³ i⁵¹⁴ n⁵¹⁵ l⁵¹⁶ h⁵¹⁷

The following Replikin sequences were identified in the carboxy terminal of Accession Number Q9WUL0:

(SEQ ID NO: 313)
h⁵¹³ i⁵¹⁴ n⁵¹⁵ l⁵¹⁶ h⁵¹⁷ p⁵¹⁸ e⁵¹⁹ l⁵²⁰ d⁵²¹ g⁵²² q⁵²³ e⁵²⁴ y⁵²⁵ v⁵²⁶ v⁵²⁷ e⁵²⁸ f⁵²⁹ d⁵³⁰ f⁵³¹
p⁵³² g⁵³³ k⁵³⁴ d⁵³⁵ s⁵³⁶ i⁵³⁷ r⁵³⁸ y⁵³⁹ y⁵⁴⁰ n⁵⁴¹ k⁵⁴²
(SEQ ID NO: 314)
h⁵¹³ i⁵¹⁴ n⁵¹⁵ l⁵¹⁶ h⁵¹⁷ p⁵¹⁸ e⁵¹⁹ l⁵²⁰ d⁵²¹ g⁵²² q⁵²³ e⁵²⁴ y⁵²⁵ v⁵²⁶ v⁵²⁷ e⁵²⁸ f⁵²⁹ d⁵³⁰ f⁵³¹
p⁵³² g⁵³³ k⁵³⁴ d⁵³⁵ s⁵³⁶ i⁵³⁷ r⁵³⁸ y⁵³⁹ y⁵⁴⁰ n⁵⁴¹ k⁵⁴² v⁵⁴³ p⁵⁴⁴ v⁵⁴⁵ e⁵⁴⁶ k⁵⁴⁷ r⁵⁴⁸ v⁵⁴⁹ f⁵⁵⁰
k⁵⁵¹
(SEQ ID NO: 315)
h⁵¹³ i⁵¹⁴ n⁵¹⁵ l⁵¹⁶ h⁵¹⁷ p⁵¹⁸ e⁵¹⁹ l⁵²⁰ d⁵²¹ g⁵²² q⁵²³ e⁵²⁴ y⁵²⁵ v⁵²⁶ v⁵²⁷ e⁵²⁸ f⁵²⁹ d⁵³⁰ f⁵³¹
p⁵³² g⁵³³ k⁵³⁴ d⁵³⁵ s⁵³⁶ i⁵³⁷ r⁵³⁸ y⁵³⁹ y⁵⁴⁰ n⁵⁴¹ k⁵⁴² v⁵⁴³ p⁵⁴⁴ v⁵⁴⁵ e⁵⁴⁶ k⁵⁴⁷ r⁵⁴⁸ v⁵⁴⁹ f⁵⁵⁰
k⁵⁵¹ n⁵⁵² l⁵⁵³ q⁵⁵⁴ l⁵⁵⁵ f⁵⁵⁶ m⁵⁵⁷ e⁵⁵⁸ n⁵⁵⁹ k⁵⁶⁰
(SEQ ID NO: 316)
h⁵¹⁷ p⁵¹⁸ e⁵¹⁹ l⁵²⁰ d⁵²¹ g⁵²² q⁵²³ e⁵²⁴ y⁵²⁵ v⁵²⁶ v⁵²⁷ e⁵²⁸ f⁵²⁹ d⁵³⁰ f⁵³¹ p⁵³² g⁵³³ k⁵³⁴ d⁵³⁵
s⁵³⁶ i⁵³⁷ r⁵³⁸ y⁵³⁹ y⁵⁴⁰ n⁵⁴¹ k⁵⁴²
(SEQ ID NO: 317)
h⁵¹⁷ p⁵¹⁸ e⁵¹⁹ l⁵²⁰ d⁵²¹ g⁵²² q⁵²³ e⁵²⁴ y⁵²⁵ v⁵²⁶ v⁵²⁷ e⁵²⁸ f⁵²⁹ d⁵³⁰ f⁵³¹ p⁵³² g⁵³³ k⁵³⁴ d⁵³⁵
s⁵³⁶ i⁵³⁷ r⁵³⁸ y⁵³⁹ y⁵⁴⁰ n⁵⁴¹ k⁵⁴² v⁵⁴³ p⁵⁴⁴ v⁵⁴⁵ e⁵⁴⁶ k⁵⁴⁷ r⁵⁴⁸ v⁵⁴⁹ f⁵⁵⁰ k⁵⁵¹
(SEQ ID NO: 318)
h⁵¹⁷ p⁵¹⁸ e⁵¹⁹ l⁵²⁰ d⁵²¹ g⁵²² q⁵²³ e⁵²⁴ y⁵²⁵ v⁵²⁶ v⁵²⁷ e⁵²⁸ f⁵²⁹ d⁵³⁰ f⁵³¹ p⁵³² g⁵³³ k⁵³⁴ d⁵³⁵
s⁵³⁶ i⁵³⁷ r⁵³⁸ y⁵³⁹ y⁵⁴⁰ n⁵⁴¹ k⁵⁴² v⁵⁴³ p⁵⁴⁴ v⁵⁴⁵ e⁵⁴⁶ k⁵⁴⁷ r⁵⁴⁸ v⁵⁴⁹ f⁵⁵⁰ k⁵⁵¹ n⁵⁵² l⁵⁵³ q⁵⁵⁴
l⁵⁵⁵f⁵⁵⁶ m⁵⁵⁷ e⁵⁵⁸ n⁵⁵⁹ k⁵⁶⁰
(SEQ ID NO: 319)
k⁵³⁴ d⁵³⁵ s⁵³⁶ i⁵³⁷ r⁵³⁸ y⁵³⁹ y⁵⁴⁰ n⁵⁴¹ k⁵⁴² v⁵⁴³ p⁵⁴⁴ v⁵⁴⁵ e⁵⁴⁶ k⁵⁴⁷ r⁵⁴⁸ v⁵⁴⁹ f⁵⁵⁰ k⁵⁵¹ n⁵⁵²
l⁵⁵³ q⁵⁵⁴ l⁵⁵⁵ f⁵⁵⁶ m⁵⁵⁷ e⁵⁵⁸ n⁵⁵⁹ k⁵⁶⁰ q⁵⁶¹ p⁵⁶² e⁵⁶³ d⁵⁶⁴ d⁵⁶⁵ l⁵⁶⁶ f⁵⁶⁷ d⁵⁶⁸ r⁵⁶⁹ l⁵⁷⁰ n⁵⁷¹
t⁵⁷² g⁵⁷³ i⁵⁷⁴ l⁵⁷⁵ n⁵⁷⁶ k⁵⁷⁷ h⁵⁷⁸
(SEQ ID NO: 320)
k⁵⁴² v⁵⁴³ p⁵⁴⁴ v⁵⁴⁵ e⁵⁴⁶ k⁵⁴⁷ r⁵⁴⁸ v⁵⁴⁹ f⁵⁵⁰ k⁵⁵¹ n⁵⁵² l⁵⁵³ q⁵⁵⁴ l⁵⁵⁵ f⁵⁵⁶ m⁵⁵⁷ e⁵⁵⁸ n⁵⁵⁹ k⁵⁶⁰
q⁵⁶¹ p⁵⁶² e⁵⁶³ d⁵⁶⁴ d⁵⁶⁵ l⁵⁶⁶ f⁵⁶⁷ d⁵⁶⁸ r⁵⁶⁹ l⁵⁷⁰ n⁵⁷¹ t⁵⁷² g⁵⁷³ i⁵⁷⁴ l⁵⁷⁵ n⁵⁷⁶ k⁵⁷⁷ h⁵⁷⁸
(SEQ ID NO: 321)
k⁵⁵¹ n⁵⁵² l⁵⁵³ q⁵⁵⁴ l⁵⁵⁵ f⁵⁵⁶ m⁵⁵⁷ e⁵⁵⁸ n⁵⁵⁹ k⁵⁶⁰ q⁵⁶¹ p⁵⁶² e⁵⁶³ d⁵⁶⁴ d⁵⁶⁵ l⁵⁶⁶ f⁵⁶⁷ d⁵⁶⁸ r⁵⁶⁹
l⁵⁷⁰ n⁵⁷¹ t⁵⁷² g⁵⁷³ i⁵⁷⁴ l⁵⁷⁵ n⁵⁷⁶ k⁵⁷⁷ h⁵⁷⁸
(SEQ ID NO: 322)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
(SEQ ID NO: 323)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵
(SEQ ID NO: 324)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵ d⁶⁶⁶ l⁶⁶⁷ k⁶⁶⁸ s⁶⁶⁹ a⁶⁷⁰ k⁶⁷¹
(SEQ ID NO: 325)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵ d⁶⁶⁶ l⁶⁶⁷ k⁶⁶⁸ s⁶⁶⁹ a⁶⁷⁰ k⁶⁷¹
a⁶⁷² d⁶⁷³ a⁶⁷⁴ k⁶⁷⁵
(SEQ ID NO: 326)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵ d⁶⁶⁶ l⁶⁶⁷ k⁶⁶⁸ s⁶⁶⁹ a⁶⁷⁰ k⁶⁷¹
a⁶⁷² d⁶⁷³ a⁶⁷⁴ k⁶⁷⁵ v⁶⁷⁶ m⁶⁷⁷ k⁶⁷⁸
(SEQ ID NO: 327)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵ d⁶⁶⁶ l⁶⁶⁷ k⁶⁶⁸ s⁶⁶⁹ a⁶⁷⁰ k⁶⁷¹
a⁶⁷² d⁶⁷³ a⁶⁷⁴ k⁶⁷⁵ v⁶⁷⁶ m⁶⁷⁷ k⁶⁷⁸ d⁶⁷⁹ a⁶⁸⁰ k⁶⁸¹
(SEQ ID NO: 328)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵ d⁶⁶⁶ l⁶⁶⁷ k⁶⁶⁸ s⁶⁶⁹ a⁶⁷⁰ k⁶⁷¹
a⁶⁷² d⁶⁷³ a⁶⁷⁴ k⁶⁷⁵ v⁶⁷⁶ m⁶⁷⁷ k⁶⁷⁸ d⁶⁷⁹ a⁶⁸⁰ k⁶⁸¹ t⁶⁸² k⁶⁸³
(SEQ ID NO: 329)
h⁶³⁴ q⁶³⁵ r⁶³⁶ a⁶³⁷ p⁶³⁸ p⁶³⁹ k⁶⁴⁰ t⁶⁴¹ f⁶⁴² e⁶⁴³ k⁶⁴⁴ s⁶⁴⁵ m⁶⁴⁶ m⁶⁴⁷ n⁶⁴⁸ l⁶⁴⁹ q⁶⁵⁰ s⁶⁵¹ k⁶⁵²
i⁶⁵³ d⁶⁵⁴ a⁶⁵⁵ k⁶⁵⁶ k⁶⁵⁷ d⁶⁵⁸ q⁶⁵⁹ l⁶⁶⁰ a⁶⁶¹ d⁶⁶² a⁶⁶³ r⁶⁶⁴ k⁶⁶⁵ d⁶⁶⁶ l⁶⁶⁷ k⁶⁶⁸ s⁶⁶⁹ a⁶⁷⁰ k⁶⁷¹
a⁶⁷² d⁶⁷³ a⁶⁷⁴ k⁶⁷⁵ v⁶⁷⁶ m⁶⁷⁷ k⁶⁷⁸ d⁶⁷⁹ a⁶⁸⁰ k⁶⁸¹ t⁶⁸² k⁶⁸³ k⁶⁸⁴

Within Accession Number Q9WUL0, which is 767 amino acids in length, 328 Replikin sequences were identified. The Replikin Count of the entire sequence was found to be (328/767)*100 or 42.8 Replikin sequences per 100 amino acid residues.

A Replikin Peak Gene was identified within the amino acid sequence of Accession Number Q9WUL0 from residue 22 through residue 95 with a length of 74 amino acids. Within the Replikin Peak Gene, 234 continuous and/or overlapping Replikin sequences were identified. The Replikin Count of the Replikin Peak Gene was calculated to be (234/74)*100 or 316 Replikin sequences per 100 amino acid residues.

Because Accession Number Q9WUL0 had the highest Replikin Count in its Replikin Peak Gene among all of the other rat glioblastoma sequences queried at www.pubmed.com (and because the glioblastoma containing the topoisomerase protein reported at Accession Number Q9WUL0 was predicted to have the relative fastest rate of growth and highest lethality), Replikin sequences from Accession Number Q9WUL0 were chosen for design of a vaccine to be tested in the rat model of glioblastoma. Rats are accepted in the art as a good model for development of human glioblastoma therapies.

A vaccine was designed by choosing thirty-two Replikin sequences from the Replikin Peak Gene wherein each of the Replikin sequences had an amino acid length of from seven to sixteen amino acid residues. Five additional Replikin sequences (from eight to fifteen amino acid residues in length) identified in the mid-molecule portion of the gene were added. Finally a Replikin sequence originally identified in human glioblastoma (kagvaflhkk (SEQ ID NO: 330)) was added. SEQ ID NO: 330 is the original prototype Replikin sequence, from which an algorithm was derived by which all other Replikin sequences were identified. The original prototype glioma Replikin Sequence, SEQ ID NO: 330, was first identified in the malignin oncoprotein in glioblastoma in humans and was observed to be related to rapid replication of glioblastoma cells. See, e.g., U.S. Pat. No. 7,420,028 for a description of the identification and isolation of SEQ ID NO: 330 as well as a description of the relationship of SEQ ID NO: 330 to rapid replication in glioblastoma cells.

The vaccine as designed contained the following thirty-eight Replikin sequences:

(SEQ ID NO: 330)
k a g v a f l h k k
(SEQ ID NO: 304)
h⁴⁰¹ k⁴⁰² w⁴⁰³ k⁴⁰⁴ e⁴⁰⁵ v⁴⁰⁶ r⁴⁰⁷ h⁴⁰⁸ d⁴⁰⁹ n⁴¹⁰ k⁴¹¹
(SEQ ID NO: 302)
k³⁹³ v³⁹⁴ p³⁹⁵ s³⁹⁶ p³⁹⁷ p³⁹⁸ p³⁹⁹ g⁴⁰⁰ h⁴⁰¹ k⁴⁰²
(SEQ ID NO: 284)
k²⁶⁴ m²⁶⁵ l²⁶⁶ d²⁶⁷ h²⁶⁸ e²⁶⁹ y²⁷⁰ t²⁷¹ t²⁷² k²⁷³
(SEQ ID NO: 283)
k²⁵⁴ a²⁵⁵ e²⁵⁶ e²⁵⁷ v²⁵⁸ a²⁵⁹ t²⁶⁰ f²⁶¹ f²⁶² a²⁶³ k²⁶⁴ m²⁶⁵ l²⁶⁶ d²⁶⁷ h²⁶⁸
(SEQ ID NO: 275)
k²¹⁸ w²¹⁹ k²²⁰ f²²¹ l²²² e²²³ h²²⁴ k²²⁵
(SEQ ID NO: 256)
h⁸³ k⁸⁴ d⁸⁵ r⁸⁶ d⁸⁷ k⁸⁸ e⁸⁹ k⁹⁰
(SEQ ID NO: 253)
k⁸² h⁸³ k⁸⁴ d⁸⁵ r⁸⁶ d⁸⁷ k⁸⁸
(SEQ ID NO: 251)
k⁸⁰ d⁸¹ k⁸² h⁸³ k⁸⁴ d⁸⁵ r⁸⁶ d⁸⁷ k⁸⁸
(SEQ ID NO: 247)
h⁷⁹ k⁸⁰ d⁸¹ k⁸² h⁸³ k⁸⁴ d⁸⁵ r⁸⁶ d⁸⁷ k⁸⁸
(SEQ ID NO: 243)
k⁷⁸ h⁷⁹ k⁸⁰ d⁸¹ k⁸² h⁸³ k⁸⁴
(SEQ ID NO: 239)
k⁷² d⁷³ g⁷⁴ s⁷⁵ s⁷⁶ d⁷⁷ k⁷⁸ h⁷⁹
(SEQ ID NO: 231)
h⁷¹ k⁷² d⁷³ g⁷⁴ s⁷⁵ s⁷⁶ d⁷⁷ k⁷⁸
(SEQ ID NO: 225)
k⁷⁰ h⁷¹ k⁷² d⁷³ g⁷⁴ s⁷⁵ s⁷⁶d⁷⁷ k⁷⁸
(SEQ ID NO: 222)
k⁶⁸ t⁶⁹ k⁷⁰ h⁷¹ k⁷² d⁷³ g⁷⁴ s⁷⁵ s⁷⁶ d⁷⁷ k⁷⁸
(SEQ ID NO: 219)
k⁶⁶ e⁶⁷ k⁶⁸ t⁶⁹ k⁷⁰ h⁷¹ k⁷²
(SEQ ID NO: 215)
k⁶⁴ e⁶⁵ k⁶⁶ e⁶⁷ k⁶⁸ t⁶⁹ k⁷⁰ h⁷¹
(SEQ ID NO: 207)
h⁶³ k⁶⁴ e⁶⁵ k⁶⁶ e⁶⁷ k⁶⁸ t⁶⁹ k⁷⁰ h⁷¹ k⁷² d⁷³ g⁷⁴ s⁷⁵ s⁷⁶ d⁷⁷ k⁷⁸
(SEQ ID NO: 197)
k⁶² h⁶³ k⁶⁴ e⁶⁵ k⁶⁶ e⁶⁷ k⁶⁸
(SEQ ID NO: 192)
k⁶¹ k⁶² h⁶³ k⁶⁴ e⁶⁵ k⁶⁶ e⁶⁷ k⁶⁸
(SEQ ID NO: 187)
k⁵⁷ d⁵⁸ s⁵⁹ e⁶⁰ k⁶¹ k⁶²h⁶³ k⁶⁴
(SEQ ID NO: 172)
h⁵⁶ k⁵⁷ d⁵⁸ s⁵⁹ e⁶⁰ k⁶¹ k⁶² h⁶³ k⁶⁴
(SEQ ID NO: 155)
k⁵⁰ h⁵¹ s⁵² n⁵³ s⁵⁴ e⁵⁵ h⁵⁶ k⁵⁷ d⁵⁸ s⁵⁹ e⁶⁰ k⁶¹ k⁶² h⁶³
(SEQ ID NO: 149)
k⁴⁸ s⁴⁹ k⁵⁰ h⁵¹ s⁵² n⁵³ s⁵⁴ e⁵⁵ h⁵⁶ k⁵⁷
(SEQ ID NO: 143)
k⁴⁴ d⁴⁵ r⁴⁶ e⁴⁷ k⁴⁸ s⁴⁹ k⁵⁰ h⁵¹
(SEQ ID NO: 137)
k⁴² d⁴³ k⁴⁴ d⁴⁵ r⁴⁶ e⁴⁷ k⁴⁸ s⁴⁹ k⁵⁰ h⁵¹
(SEQ ID NO: 131)
k⁴⁰ d⁴¹ k⁴² d⁴³ k⁴⁴ d⁴⁵ r⁴⁶ e⁴⁷ k⁴⁸ s⁴⁹ k⁵⁰ h⁵¹
(SEQ ID NO: 125)
k³⁹ k⁴⁰ d⁴¹ k⁴² d⁴³ k⁴⁴ d⁴⁵ r⁴⁶ e⁴⁷ k⁴⁸ s⁴⁹ k⁵⁰ h⁵¹
(SEQ ID NO: 113)
h³⁸ k³⁹ k⁴⁰ d⁴¹ k⁴² d⁴³ k⁴⁴ d⁴⁵ r⁴⁶ e⁴⁷ k⁴⁸
(SEQ ID NO: 104)
k³⁶ e³⁷ h³⁸ k³⁹ k⁴⁰ d⁴¹ k⁴²
(SEQ ID NO: 90)
h³⁵ k³⁶ e³⁷ h³⁸ k³⁹ k⁴⁰ d⁴¹ k⁴²
(SEQ ID NO: 77)
h³³ r³⁴ h³⁵ k³⁶ e³⁷ h³⁸ k³⁹ k⁴⁰ d⁴¹ k⁴²
(SEQ ID NO: 69)
k²⁹ d³⁰ r³¹ e³² h³³ r³⁴ h³⁵ k³⁶
(SEQ ID NO: 56)
h²⁸ k²⁹ d³⁰ r³¹ e³² h³³ r³⁴ h³⁵ k³⁶
(SEQ ID NO: 50)
k²⁷ h²⁸ k²⁹ d³⁰ r³¹ e³² h³³ r³⁴ h³⁵ k³⁶
(SEQ ID NO: 38)
h²⁴ k²⁵ d²⁶ k²⁷ h²⁸ k²⁹ d³⁰ r³¹ e³² h³³ r³⁴ h³⁵ k³⁶
(SEQ ID NO: 34)
k²³ h²⁴ k²⁵ d²⁶ k²⁷ h²⁸ k²⁹
(SEQ ID NO: 17)

h²² k²³ h²⁴ k²⁵ d²⁶ k²⁷ h²⁸ k²⁹

A vaccine comprising the thirty-eight Replikin sequences and a pharmaceutically acceptable carrier is administered to rats either intravenously or intramuscularly. An immune response is detected. The vaccine is then used as a therapy against growth of glioblastoma in rats.

### Example 6

### Survey of Glioblastoma Tissue to Develop Baseline Data for Case-by-Case Prognosis

More than 500 frozen tissue samples from glioblastoma tumors are analyzed to determine the Replikin Count in the topoisomerase gene in each tumor. A Replikin Peak Gene is identified in each topoisomerase gene. The Replikin Count in each Replikin Peak Gene is recorded and compared with the measured lethality of the malignancy in the patient suffering from each tumor and/or the previously recorded histopathologically-determined degree of malignancy for each tumor. A mean Replikin Count plus standard deviation is determined among the more than 500 samples or a correlation between the Replikin Count and the measured lethality of each malignancy is determined by any method. Such method may include, for example, a Replikin Count Expansion Index analysis or a regression analysis or any other method of analysis or the histopathologically-determined degree of malignancy.

A Replikin Count of a malignancy having an unknown lethality may then be compared to the mean Replikin Count plus one standard deviation of the mean. If the Replikin Count of the malignancy having an unknown lethality is greater than the mean Replikin Count plus one standard deviation of the mean, the malignancy of unknown lethality is predicted to have a greater lethality than the mean measured lethality of the more than 500 samples. If the Replikin Count of the malignancy having an unknown lethality is less than the mean Replikin Count minus one standard deviation of the mean, the malignancy of unknown lethality is predicted to have a lower lethality than the mean measured lethality of the more than 500 samples. To provide other kinds of precision in the prediction of lethality, the more than 500 samples may be grouped into a plurality of groups having related lethalities or related types. Mean Replikin Counts plus standard deviation of the mean may be determined for some or all of each these groups. A Replikin Count of a malignancy having an unknown lethality may then be compared to the mean Replikin Count plus or minus one standard deviation of the mean of a particular group having a related lethality.

A regression analysis may also be performed on the Replikin Counts of the more than 500 samples where Replikin Count is compared to measured lethality. The regression analysis may be used to create a predictive formula by which Replikin Count in the Replikin Peak Gene of the topoisomerase gene in individual cases of glioblastoma may be used to provide a predicted lethality prognosis for any biopsy or resection of a glioblastoma growth in a patient suffering from glioblastoma. The statistical formula provides a measure of lethality such as length of life expectancy or expected rate of growth based on the Replikin Count of the Replikin Peak Gene of the topoisomerase of the glioblastoma.

Two or more regression curves may be applied to data from a survey. For example, among the more than 500 samples, samples may be grouped into benign and malignant tumors. A first regression analysis may be performed on the group of benign tumors and a second regression analysis may be performed on the group of malignant tumors. Further, a regression curve may be continuous or broken. It further may reflect types or subtypes of malignancies.

The efficacy of histopathologically-determined degree of malignancy is also determined by comparing the histopathological degree of malignancy previously qualitatively determined by pathologists with a quantitative measure of lethality based on quantitative Replikin Counts.

Additional data is created for other proteins expressed in glioblastoma. The protein, protein fragment, or genome segment of the glioblastoma that provides the strongest correlation is chosen as a standard for determination of lethality using Replikin Count.

A kit comprising the Replikin Count Expansion Index or predictive formula for determining prognosis based on Replikin Count is provided to laboratories and practitioners so that prognosis based on Replikin sequence analysis of individual malignancies may be provided directly from the practitioner or laboratory to the patient. The kit may comprise software containing the Replikin Count Expansion Index or regression formula.

### Example 7

### Comparison of the Replikin Concentration of Four Strains of Taura Syndrome Virus by an Independent Laboratory

The Replikin concentrations of the expressed protein sequences of four taura syndrome virus (TSV) isolates from Hawaii, Belize, Thailand and Venezuela, respectively, were examined. The virulence of each isolate was initially quantitatively ranked based solely on the order of the Replikin concentrations. This quantitative ranking of virulence based on Replikin concentration was undertaken by researchers having no knowledge of the virulence of the isolates based on bioassay methods (such as cumulative survival and time to 50% mortality) undertaken simultaneously.

An independent laboratory simultaneously undertook bioassay comparisons of the four TSV isolates. The independent laboratory had no knowledge of the order of the Replikin concentrations of the TSV isolates. In the independent laboratory, virulence was compared in infected Litopenaeus vannamei (Kona stock, Oceanic Institute, Hawaii) shrimp subject to per os viral infection. Cumulative survival results and time to 50% mortality results demonstrated the Belize isolate to be the most virulent, the Thailand isolate to be the second most virulent, the Hawaii isolate to be the third most virulent, and the Venezuela isolate to be the least virulent. TSV infection was confirmed as the cause of death in each bioassay by positive reactions in RT-PCR detection and by the appearance of characteristic lesions observed in histological analysis. A full description of the investigative methods of the shrimp trials is set forth in Example 1 of U.S. patent application Ser. No. 12/108,458 filed Apr. 23, 2008.

Upon comparison of Replikin concentration for each isolate with cumulative survival of challenged shrimp and 50% mortality of challenged shrimp, a quantitative and substantially linear correlation was observed. The results are set forth in Table 2 below.

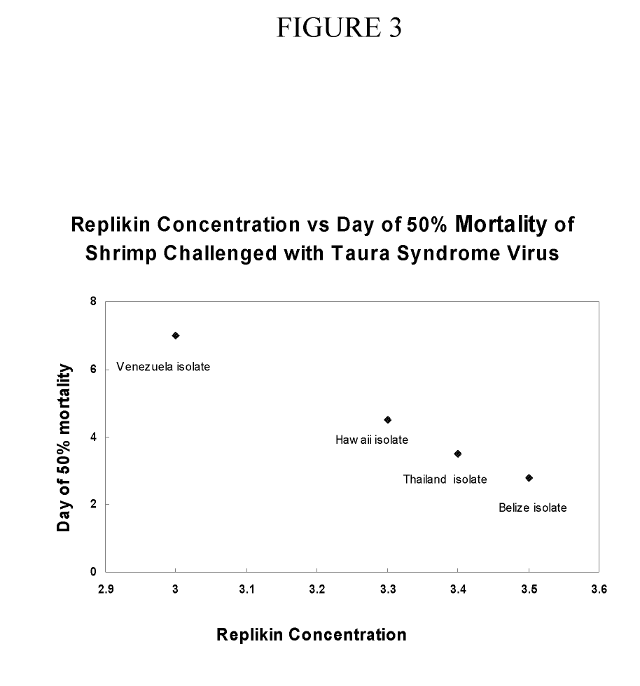

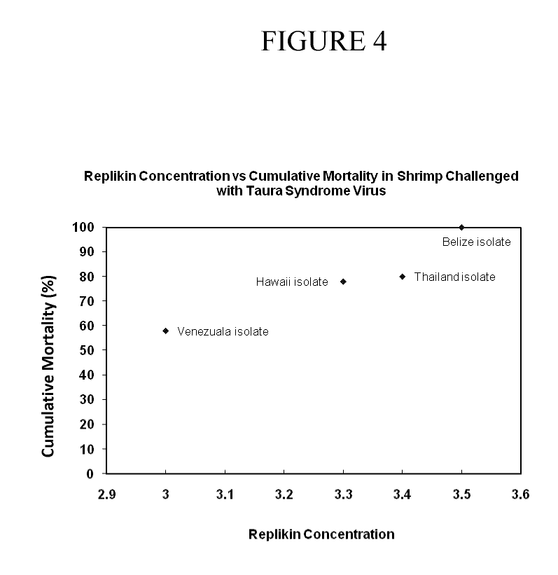

Table 2 provides the time to 50% mortality for shrimp challenged with each isolate. Fifty percent mortality resulting from TSV infection with the isolate of Belize, Thailand, Hawaii and Venezuela, respectively, were 2.8, 3.5, 4.5 and 7 days. Table 2 also provides the cumulative mortality of shrimp fifteen days following challenge. Cumulative mortality at fifteen days for shrimp challenged with the Belize, Thailand, Hawaii, and Venezuelans isolates, respectively, was 100%, 80%, 78%, and 58%, respectively. Statistical differences between the Replikin concentration for each isolate are significant at a level of p<0.001. FIGS. 3 and 4 provide graphical illustration of the data in Table 2.

TABLE 2
Results from per os TSV challenge in SPF Litopenaeus vannamei
(Kona stock)

| | GenBank | Cumulative | | |
|---|---|---|---|---|
| | No. | Mortality | Day of 50% | Blind Replikin |
| TSV isolate | (ORF1) | (%)(Mean) | mortality | Concentration |
| Belize | AAT81157 | 100 | 2.8 | 3.5 |
| Thailand | AAY56363 | 80 | 3.5 | 3.4 |
| US-Hawaii | AAK72220 | 78 | 4.5 | 3.3 |
| Venezuela | ABB17263 | 58 | 7.0 | 3.0 |

What is claimed is:

1. A method of predicting the relative rate of growth of at least one first malignancy of an unknown or known type of malignancy comprising:
comparing a Replikin Count of the at least one first malignancy with a Replikin Count of at least one second malignancy of an unknown type or of a different type of malignancy than the at least one first malignancy; and
predicting the at least one first malignancy to have a relative rate of growth that is faster than the relative rate of growth of the at least one second malignancy if the Replikin Count of the at least one first malignancy is greater than the Replikin Count of the at least one second malignancy.

2. The method of claim 1, further comprising: determining the Replikin Count of the at least one first malignancy and the Replikin Count of the at least one second malignancy before the comparing step.

3. The method of claim 1, wherein said at least one first malignancy is a metastasis of said at least one second malignancy, is a malignancy of unknown type or unknown origin in a subject suffering from said at least one second malignancy, or is a malignant cell of the same malignancy as said at least one second malignancy, or wherein said at least one first malignancy and at least one second malignancy are metastases of a third malignancy.

4. The method of claim 1, wherein the Replikin Count of said at least one first malignancy is the Replikin Count of a Replikin Peak Gene of the at least one first malignancy and the Replikin Count of said at least one second malignancy is a Replikin Count of a Replikin Peak Gene of said at least one second malignancy.

5. The method of claim 1, wherein (1) said Replikin Count of said at least one second malignancy is a mean Replikin Count of a plurality of malignancies or a mean Replikin Count of a plurality of cells of the at least one second malignancy; (2) said Replikin Count of said at least one first malignancy is a mean Replikin Count of a plurality of malignancies or a mean Replikin Count of a plurality of cells of the at least one first malignancy; or (3) both said Replikin Count of said at least one second malignancy is a mean Replikin Count of a plurality of malignancies or a mean Replikin Count of a plurality of cells of the at least one second malignancy, and said Replikin Count of said at least one first malignancy is a mean Replikin Count of a plurality of malignancies or a mean Replikin Count of a plurality of cells of the at least one first malignancy.

6. The method of claim 1, wherein said Replikin Count of said at least one second malignancy is a mean, median, or other average Replikin Count of a plurality of malignancies, wherein said plurality of malignancies represents a survey of malignancies.

7. The method of claim 5, wherein the Replikin Count of said at least one first malignancy or the mean Replikin Count of said at least one first malignancy is greater than a mean Replikin Count of said at least one second malignancy plus the standard deviation of the mean Replikin Count of said at least one second malignancy.

8. The method of claim 1, wherein said relative rate of growth correlates with a relative lethality.

9. The method of claim 1, wherein said Replikin Count of said at least one first malignancy is a Replikin Count of an expressed protein, protein fragment or peptide isolated from a cell of said at least one first malignancy or a Replikin Count of the genome, a gene, a gene fragment, or any other nucleic acid sequence isolated from a cell of said at least one first malignancy; and wherein said Replikin Count of said at least one second malignancy is a Replikin Count of an expressed protein, protein fragment or peptide isolated from a cell of said at least one second malignancy or a Replikin Count of the genome, a gene, a gene fragment, or any other nucleic acid sequence isolated from a cell of said at least one second malignancy.

10. The method of claim 1, wherein said Replikin Count of said at least one first malignancy is the highest Replikin Count among a plurality of Replikin Counts of a first plurality of malignancies of a first type, and said Replikin Count of said at least one second malignancy is the highest Replikin Count among a plurality of Replikin Counts of a second plurality of malignancies of a second type.

11. The method of claim 10, further comprising: comparing a Replikin Count of the Replikin Peak Gene of the malignancy having the highest Replikin Count among a plurality of Replikin Counts of said malignancies of the first type to the Replikin Count of the Replikin Peak Gene of the malignancy having the highest Replikin Count among a plurality of Replikin Counts of said malignancies of the second type; and if said malignancies of the first type has a higher Replikin Count of said Replikin Peak Gene than said malignancies of the second type, then said malignancies of the first type is predicted to have a greater lethality than said malignancies of the second type.

12. The method of claim 1, wherein said at least one first malignancy is a thyroid malignancy, a prostate malignancy, a breast malignancy, a urinary bladder malignancy, a uterine corpus malignancy, a uterine cervix malignancy, a colon malignancy, an ovarian malignancy, a malignancy of the oral cavity, a lymphocytic leukemia malignancy, a multiple myeloma malignancy, a gastric malignancy, a non-small cell lung carcinoma malignancy, or a glioblastoma malignancy.

13. The method of claim 1, wherein the method is performed by a processor and wherein at least one Replikin Count or at least one representation of said prediction of the relative rate of growth of said at least one first malignancy is outputted to a user or display.

14. A machine-readable storage medium having stored thereon executable instructions that, when executed by a processor, cause the processor to provide sufficient data to a user, a display, or a printout such that said user or a user of said display or said printout may predict the relative rate of growth of the at least one first malignancy according to the method of claim 1.

15. A computer system, comprising: a processor coupled to a network; a memory coupled to the processor, the memory containing a plurality of instructions to perform the method described by claim 1.

16. A method of making a composition comprising at least one component of at least one first malignancy, wherein said at least one first malignancy is predicted, according to the method of claim 1, to have a relative rate of growth faster than the relative rate of growth of the at least one second malignancy, wherein said method comprises including the at least one component in a composition.

17. The method of claim 16, wherein said at least one component of said at least one first malignancy is at least one Replikin peptide or at least one Replikin Peak Gene identified within or isolated from said at least one first malignancy.

18. The method of claim 16, wherein said composition is an immunogenic composition.

19. The method of claim 18, wherein said immunogenic composition is a vaccine.

20. The method of claim 16, further comprising: combining a pharmaceutically acceptable carrier, or adjuvant, or both with the at least one component of said at least one first malignancy.

21. The method of claim 17, further comprising: isolating or synthesizing said at least one Replikin peptide or said at least one Replikin Peak Gene.

22. Use of at least one component of a cell of at least one first malignancy predicted, according to the method of claim 1, to have a relative rate of growth that is faster than the relative rate of growth of the at least one second malignancy for the treatment of said at least one first or second malignancy or for the treatment of a metastatic malignancy related to said at least one first or second malignancy.

23. Use of at least one component of a cell of at least one first malignancy predicted, according to the method of claim 1, to have a relative rate of growth that is faster than the relative rate of growth of the at least one second malignancy in the manufacture of a medicament for the treatment of malignancy.

24. The use of claim 23, wherein said treatment of malignancy is the treatment of said at least one first malignancy or said at least one second malignancy or a metastatic malignancy of said at least one first malignancy or said at least one second malignancy.

25. The use of claim 22, wherein said at least one component of said cell is at least one Replikin peptide or at least one Replikin Peak Gene isolated from or identified in said cell.

26. The use of claim 23, wherein said at least one component of said cell is at least one Replikin peptide or at least one Replikin Peak Gene isolated from or identified in said cell.

27. The use of claim 24, wherein said at least one component of said cell is at least one Replikin peptide or at least one Replikin Peak Gene isolated from or identified in said cell.

28. A method of treating a malignancy comprising administering to a subject at least one component of a cell of at least one first malignancy predicted, according to the method of claim 1, to have a relative rate of growth that is faster than the relative rate of growth of at least one second malignancy.

29. A method of stimulating the immune system of a subject comprising administering to said subject, at least one component of a cell of at least one first malignancy predicted, according to the method of claim 1, to have a relative rate of growth that is faster than the relative rate of growth of at least one second malignancy.

30. A method of making an antibody or an antibody fragment that binds to at least one component of a cell of at least one first malignancy, wherein said at least one first malignancy is predicted, according to the method of claim 1, to have a relative rate of growth that is faster than the relative rate of growth of at least one second malignancy comprising identifying said at least one Replikin peptide or said at least one Replikin Peak Gene and making an antibody or antibody fragment that binds to said at least one Replikin peptide or said at least one Replikin Peak Gene.

31. The method of claim 28, wherein said at least one component of said cell is at least one Replikin peptide or at least one Replikin Peak Gene isolated from or identified in said cell.

32. The method of claim 29, wherein said at least one component of said cell is at least one Replikin peptide or at least one Replikin Peak Gene isolated from or identified in said cell.

33. The method of claim 30, wherein said at least one component of said cell is at least one Replikin peptide or at least one Replikin Peak Gene isolated from or identified in said cell.

34. A method of making at least one siRNA comprising: identifying at least one Replikin peptide or at least one Replikin Peak Gene in at least one first malignancy predicted, according to the method of claim 1, to have a relative rate of growth that is faster than the relative rate of growth of at least one second malignancy; and making said at least one siRNA to be complementary to at least a portion of a nucleic acid that encodes said at least one Replikin peptide or said at least one Replikin Peak Gene.

35. A method of determining the relative rate of increase in the relative rate of growth of at least one first malignancy as compared to the relative rate of increase in the relative rate of growth of at least one second malignancy comprising: comparing the standard deviation of the mean Replikin Count of a plurality of cells of said at least one first malignancy to the standard deviation of the mean Replikin Count of a plurality of cells of said at least one second malignancy; and predicting that the relative increase in the relative rate of growth of said at least one first malignancy is greater than the relative rate of increase in the relative rate of growth of said at least one second malignancy if the standard deviation of the mean Replikin Count of the plurality of cells of said at least one first malignancy is greater than the standard deviation of the mean Replikin Count of a plurality of cells of said at least one second malignancy.

36. The method of claim 35, wherein said at least one first malignancy is a metastasis of said at least one second malignancy or wherein said at least one first malignancy is a malignancy of unknown origin or unknown type in a subject suffering from said at least one second malignancy.

37. The method of claim 35, wherein said at least one first malignancy and said at least one second malignancy are both metastases of a third malignancy.

38. The method of claim 35, wherein said at least one first malignancy is a plurality of malignancies differing from said at least one second malignancy, said at least one second malignancy is a plurality of malignancies differing from said at least one first malignancy, or said at least one first malignancy is a plurality of malignancies differing from said at least one second malignancy and said at least one second malignancy is a plurality of malignancies differing from said at least one first malignancy.

39. The method of claim 38, wherein said at least one first malignancy is a plurality of malignancies of a first type of malignancy and said at least one second malignancy is a plurality of malignancies of a second type of malignancy.

40. A method of predicting an expansion of a malignancy comprising:
determining a mean Replikin Count and a standard deviation of said mean Replikin Count for a plurality of cells of a first malignancy for a first time period in a first anatomic region or for a first plurality of malignancies;
determining a Replikin Count of at least one other cell of the first malignancy, at least one cell of a metastasis of the first malignancy, or at least one cell of a malignancy of unknown origin or unknown type in a subject suffering from the first malignancy at a second time period and/or in a second anatomic region, wherein said second time period is different from said first time period and/or said second anatomic region is different from said first anatomic region, or determining a Replikin Count of at least one cell of a second malignancy in a different subject; and
predicting an expansion of said first malignancy, said metastasis of said first malignancy, said malignancy of unknown origin or unknown type, or said second malignancy in a different subject if the Replikin Count of said at least one cell is greater than the mean Replikin Count of the plurality of cells of said malignancy from said first time period and from said first anatomic region plus one standard deviation of the mean Replikin Count of the plurality of cells of said malignancy from said first time period and from said first anatomic region or if the Replikin Count of said at least one cell is greater than the mean Replikin Count of the first plurality of malignancies plus one standard deviation of the mean Replikin Count of the first plurality of malignancies.

41. The method of claim 40, wherein said at least one other cell of the first malignancy, at least one cell of a metastasis of the first malignancy, or at least one cell of a malignancy of unknown origin or unknown type in a subject suffering from the first malignancy at a second time period and/or in a second anatomic region, or said at least one cell of a second malignancy in a different subject is a plurality of cells from said second time period and/or said second anatomic region or said second malignancy in a different subject, and the Replikin Count of each cell of the plurality of cells from said second time period and/or second anatomic region or said second malignancy in a different subject is compared separately to said mean Replikin Count plus one standard deviation of said mean Replikin Count.

42. The method of claim 41, wherein the expansion of said malignancy in said second time period and/or said second anatomic region or the expansion of said second malignancy in a different subject is predicted if the number of Replikin Counts of said plurality of cells from said second period and/or said second anatomic region or the number of Replikin Counts of said second malignancy in a different subject that is greater than said mean Replikin Count of the plurality of cells from said first time period in said first anatomic region or from said first plurality of malignancies plus one standard deviation of the mean of the Replikin Count is greater than the number of Replikin Counts of said plurality of cells from said second time period and/or said second anatomic region or the number of Replikin Counts of said second malignancy in a different subject that is less than said mean Replikin Count of said plurality of cells from said first time period in said first anatomic region or from said first plurality of malignancies minus one standard deviation of the mean.

43. A kit for providing a prognosis of a patient suffering from a malignancy, said prognosis concerning the lethality of the malignancy, comprising a formula for determining the lethality of a malignancy based on a Replikin Count in at least one cell of the malignancy.

44. The kit of claim 43, wherein said formula is derived from a survey of a plurality of malignancies of a first type of malignancy.

45. The kit of claim 44 wherein the first type of malignancy is a glioblastoma.

46. A method of predicting the lethality of a first malignancy comprising:
determining a Replikin Count of a first malignancy;
performing a regression analysis with the Replikin Count of a plurality of second malignancies versus the lethality of said plurality of second malignancies; and
predicting the lethality of the first malignancy based on the regression analysis of the plurality of second malignancies.

47. The method of claim 46, wherein the regression analysis of the plurality of second malignancies is performed using the Replikin Count of each individual second malignancy of the plurality of second malignancies and a patient survival outcome for each individual second malignancy.

48. The method of claim 47, wherein the patient outcome is a length of survival of the patient following diagnosis of malignancy.


SEQUENCE LISTING (330 sequences)

SEQ ID NO: 1 - protein, 767 aa, Rattus norvegicus
MSGDHLHNDSQIEADFRLNDSHKHKDKHKDREHRHKEHKKDKDKDREKSKHSNSEHKDSEKKHKEKEKTKHKDGSSDKHKDKHKDRDKEKRKEEKIRAAGDAKIKKEKENGFSSPPRIKDEPEDDGYFAPPKEDIKPLKRPRDEDDADYKPKKIKTEDIKKEKKRKLEEEEDGKLKKPKNKDKDKKVAEPDNKKKKAKKEEEQKWKWWEEERYPEGIKWKFLEHKGPVFAPPYEPLPEGVKFYYDGKVMKLSPKAEEVATFFAKMLDHEYTTKEIFRKNFFKDWRKEMTNDEKNTITNLSKCDFTQMSQYFKAQSEARKQMSKEEKLKIKEENEKLLKEYGFCVMDNHRERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFPGKDSIRYYNKVPVEKRVFKNLQLFMENKQPEDDLFDRLNTGILNKHLQDLMEGLTAKVFRTYNASITLQQQLKELTAPDENVPAKILSYNRANRAVAILCNHQRAPPKTFEKSMMNLQSKIDAKKDQLADARKDLKSAKADAKVMKDAKTKKVVESKKKAVQRLEEQLMKLEVQATDREENKQIALGTSKLNYLDPRITVAWCKKWGVPIEKIYNKTQREKFAWAIDMTDEDYEF

SEQ ID NO: 2 - protein, 25 aa, Rattus norvegicus
HLHNDSQIEADFRLNDSHKHKDKHK

SEQ ID NO: 3 - protein, 32 aa, Rattus norvegicus
HLHNDSQIEADFRLNDSHKHKDKHKDREHRHK

SEQ ID NO: 4 - protein, 35 aa, Rattus norvegicus
HLHNDSQIEADFRLNDSHKHKDKHKDREHRHKEHK

SEQ ID NO: 5 - protein, 38 aa, Rattus norvegicus
HLHNDSQIEADFRLNDSHKHKDKHKDREHRHKEHKKDK

SEQ ID NO: 6 - protein, 40 aa, Rattus norvegicus
HLHNDSQIEADFRLNDSHKHKDKHKDREHRHKEHKKDKDK

SEQ ID NO: 7 - protein, 44 aa, Rattus norvegicus
HLHNDSQIEADFRLNDSHKHKDKHKDREHRHKEHKKDKDKDREK

SEQ ID NO: 8 - protein, 46 aa, Rattus norvegicus
HLHNDSQIEADFRLNDSHKHKDKHKDREHRHKEHKKDKDKDREKSK

SEQ ID NO: 9 - protein, 23 aa, Rattus norvegicus
HNDSQIEADFRLNDSHKHKDKHK

SEQ ID NO: 10 - protein, 30 aa, Rattus norvegicus
HNDSQIEADFRLNDSHKHKDKHKDREHRHK

SEQ ID NO: 11 - protein, 33 aa, Rattus norvegicus
HNDSQIEADFRLNDSHKHKDKHKDREHRHKEHK

SEQ ID NO: 12 - protein, 36 aa, Rattus norvegicus
HNDSQIEADFRLNDSHKHKDKHKDREHRHKEHKKDK

SEQ ID NO: 13 - protein, 38 aa, Rattus norvegicus
HNDSQIEADFRLNDSHKHKDKHKDREHRHKEHKKDKDK

SEQ ID NO: 14 - protein, 42 aa, Rattus norvegicus
HNDSQIEADFRLNDSHKHKDKHKDREHRHKEHKKDKDKDREK

SEQ ID NO: 15 - protein, 44 aa, Rattus norvegicus
HNDSQIEADFRLNDSHKHKDKHKDREHRHKEHKKDKDKDREKSK

SEQ ID NO: 16 - protein, 51 aa, Rattus norvegicus
HNDSQIEADFRLNDSHKHKDKHKDREHRHKEHKKDKDKDREKSKHSNSEHK

SEQ ID NO: 17 - protein, 8 aa, Rattus norvegicus
HKHKDKHK

SEQ ID NO: 18 - protein, 15 aa, Rattus norvegicus
HKHKDKHKDREHRHK

SEQ ID NO: 19 - protein, 18 aa, Rattus norvegicus
HKHKDKHKDREHRHKEHK

SEQ ID NO: 20 - protein, 21 aa, Rattus norvegicus
HKHKDKHKDREHRHKEHKKDK

SEQ ID NO: 21 - protein, 23 aa, Rattus norvegicus
HKHKDKHKDREHRHKEHKKDKDK

SEQ ID NO: 22 - protein, 27 aa, Rattus norvegicus
HKHKDKHKDREHRHKEHKKDKDKDREK

SEQ ID NO: 23 - protein, 29 aa, Rattus norvegicus
HKHKDKHKDREHRHKEHKKDKDKDREKSK

SEQ ID NO: 24 - protein, 36 aa, Rattus norvegicus
HKHKDKHKDREHRHKEHKKDKDKDREKSKHSNSEHK

SEQ ID NO: 25 - protein, 43 aa, Rattus norvegicus
HKHKDKHKDREHRHKEHKKDKDKDREKSKHSNSEHKDSEKKHK

SEQ ID NO: 26 - protein, 45 aa, Rattus norvegicus
HKHKDKHKDREHRHKEHKKDKDKDREKSKHSNSEHKDSEKKHKEK

SEQ ID NO: 27 - protein, 47 aa, Rattus norvegicus
HKHKDKHKDREHRHKEHKKDKDKDREKSKHSNSEHKDSEKKHKEKEK

SEQ ID NO: 28 - protein, 49 aa, Rattus norvegicus
HKHKDKHKDREHRHKEHKKDKDKDREKSKHSNSEHKDSEKKHKEKEKTK

SEQ ID NO: 29 - protein, 51 aa, Rattus norvegicus
HKHKDKHKDREHRHKEHKKDKDKDREKSKHSNSEHKDSEKKHKEKEKTKHK

SEQ ID NO: 30 - protein, 29 aa, Rattus norvegicus
KHKDKHKDREHRHKEHKKDKDKDREKSKH

SEQ ID NO: 31 - protein, 34 aa, Rattus norvegicus
KHKDKHKDREHRHKEHKKDKDKDREKSKHSNSEH

SEQ ID NO: 32 - protein, 41 aa, Rattus norvegicus
KHKDKHKDREHRHKEHKKDKDKDREKSKHSNSEHKDSEKKH

SEQ ID NO: 33 - protein, 49 aa, Rattus norvegicus
KHKDKHKDREHRHKEHKKDKDKDREKSKHSNSEHKDSEKKHKEKEKTKH

SEQ ID NO: 34 - protein, 7 aa, Rattus norvegicus
KHKDKHK

SEQ ID NO: 35 - protein, 11 aa, Rattus norvegicus
KHKDKHKDREH

SEQ ID NO: 36 - protein, 13 aa, Rattus norvegicus
KHKDKHKDREHRH

SEQ ID NO: 37 - protein, 16 aa, Rattus norvegicus
KHKDKHKDREHRHKEH

SEQ ID NO: 38 - protein, 13 aa, Rattus norvegicus
HKDKHKDREHRHK

SEQ ID NO: 39 - protein, 16 aa, Rattus norvegicus
HKDKHKDREHRHKEHK

SEQ ID NO: 40 - protein, 19 aa, Rattus norvegicus
HKDKHKDREHRHKEHKKDK

SEQ ID NO: 41 - protein, 21 aa, Rattus norvegicus
HKDKHKDREHRHKEHKKDKDK

SEQ ID NO: 42 - protein, 25 aa, Rattus norvegicus
HKDKHKDREHRHKEHKKDKDKDREK

SEQ ID NO: 43 - protein, 27 aa, Rattus norvegicus
HKDKHKDREHRHKEHKKDKDKDREKSK

SEQ ID NO: 44 - protein, 34 aa, Rattus norvegicus
HKDKHKDREHRHKEHKKDKDKDREKSKHSNSEHK

SEQ ID NO: 45 - protein, 41 aa, Rattus norvegicus
HKDKHKDREHRHKEHKKDKDKDREKSKHSNSEHKDSEKKHK

SEQ ID NO: 46 - protein, 43 aa, Rattus norvegicus
HKDKHKDREHRHKEHKKDKDKDREKSKHSNSEHKDSEKKHKEK

SEQ ID NO: 47 - protein, 45 aa, Rattus norvegicus
HKDKHKDREHRHKEHKKDKDKDREKSKHSNSEHKDSEKKHKEKEK

SEQ ID NO: 48 - protein, 47 aa, Rattus norvegicus
HKDKHKDREHRHKEHKKDKDKDREKSKHSNSEHKDSEKKHKEKEKTK

SEQ ID NO: 49 - protein, 49 aa, Rattus norvegicus
HKDKHKDREHRHKEHKKDKDKDREKSKHSNSEHKDSEKKHKEKEKTKHK

SEQ ID NO: 50 - protein, 10 aa, Rattus norvegicus
KHKDREHRHK

SEQ ID NO: 51 - protein, 12 aa, Rattus norvegicus
KHKDREHRHKEH

SEQ ID NO: 52 - protein, 25 aa, Rattus norvegicus
KHKDREHRHKEHKKDKDKDREKSKH

SEQ ID NO: 53 - protein, 30 aa, Rattus norvegicus
KHKDREHRHKEHKKDKDKDREKSKHSNSEH

SEQ ID NO: 54 - protein, 37 aa, Rattus norvegicus
KHKDREHRHKEHKKDKDKDREKSKHSNSEHKDSEKKH

SEQ ID NO: 55 - protein, 45 aa, Rattus norvegicus
KHKDREHRHKEHKKDKDKDREKSKHSNSEHKDSEKKHKEKEKTKH

SEQ ID NO: 56 - protein, 9 aa, Rattus norvegicus
HKDREHRHK

SEQ ID NO: 57 - protein, 12 aa, Rattus norvegicus
HKDREHRHKEHK

SEQ ID NO: 58 - protein, 15 aa, Rattus norvegicus
HKDREHRHKEHKKDK

SEQ ID NO: 59 - protein, 17 aa, Rattus norvegicus
HKDREHRHKEHKKDKDK

SEQ ID NO: 60 - protein, 21 aa, Rattus norvegicus
HKDREHRHKEHKKDKDKDREK

SEQ ID NO: 61 - protein, 23 aa, Rattus norvegicus
HKDREHRHKEHKKDKDKDREKSK

SEQ ID NO: 62 - protein, 30 aa, Rattus norvegicus
HKDREHRHKEHKKDKDKDREKSKHSNSEHK

SEQ ID NO: 63 - protein, 37 aa, Rattus norvegicus
HKDREHRHKEHKKDKDKDREKSKHSNSEHKDSEKKHK

SEQ ID NO: 64 - protein, 39 aa, Rattus norvegicus
HKDREHRHKEHKKDKDKDREKSKHSNSEHKDSEKKHKEK

SEQ ID NO: 65 - protein, 41 aa, Rattus norvegicus
HKDREHRHKEHKKDKDKDREKSKHSNSEHKDSEKKHKEKEK

SEQ ID NO: 66 - protein, 43 aa, Rattus norvegicus
HKDREHRHKEHKKDKDKDREKSKHSNSEHKDSEKKHKEKEKTK

SEQ ID NO: 67 - protein, 45 aa, Rattus norvegicus
HKDREHRHKEHKKDKDKDREKSKHSNSEHKDSEKKHKEKEKTKHK

SEQ ID NO: 68 - protein, 51 aa, Rattus norvegicus
HKDREHRHKEHKKDKDKDREKSKHSNSEHKDSEKKHKEKEKTKHKDGSSDK

SEQ ID NO: 69 - protein, 8 aa, Rattus norvegicus
KDREHRHK

SEQ ID NO: 70 - protein, 10 aa, Rattus norvegicus
KDREHRHKEH

SEQ ID NO: 71 - protein, 11 aa, Rattus norvegicus
KDREHRHKEHK

SEQ ID NO: 72 - protein, 23 aa, Rattus norvegicus
KDREHRHKEHKKDKDKDREKSKH

SEQ ID NO: 73 - protein, 28 aa, Rattus norvegicus
KDREHRHKEHKKDKDKDREKSKHSNSEH

SEQ ID NO: 74 - protein, 35 aa, Rattus norvegicus
KDREHRHKEHKKDKDKDREKSKHSNSEHKDSEKKH

SEQ ID NO: 75 - protein, 43 aa, Rattus norvegicus
KDREHRHKEHKKDKDKDREKSKHSNSEHKDSEKKHKEKEKTKH

SEQ ID NO: 76 - protein, 51 aa, Rattus norvegicus
KDREHRHKEHKKDKDKDREKSKHSNSEHKDSEKKHKEKEKTKHKDGSSDKH

SEQ ID NO: 77 - protein, 10 aa, Rattus norvegicus
HRHKEHKKDK

SEQ ID NO: 78 - protein, 12 aa, Rattus norvegicus
HRHKEHKKDKDK

SEQ ID NO: 79 - protein, 16 aa, Rattus norvegicus
HRHKEHKKDKDKDREK

SEQ ID NO: 80 - protein, 18 aa, Rattus norvegicus
HRHKEHKKDKDKDREKSK

SEQ ID NO: 81 - protein, 25 aa, Rattus norvegicus
HRHKEHKKDKDKDREKSKHSNSEHK

SEQ ID NO: 82 - protein, 32 aa, Rattus norvegicus
HRHKEHKKDKDKDREKSKHSNSEHKDSEKKHK

SEQ ID NO: 83 - protein, 34 aa, Rattus norvegicus
HRHKEHKKDKDKDREKSKHSNSEHKDSEKKHKEK

SEQ ID NO: 84 - protein, 36 aa, Rattus norvegicus
HRHKEHKKDKDKDREKSKHSNSEHKDSEKKHKEKEK

SEQ ID NO: 85 - protein, 38 aa, Rattus norvegicus
HRHKEHKKDKDKDREKSKHSNSEHKDSEKKHKEKEKTK

SEQ ID NO: 86 - protein, 40 aa, Rattus norvegicus
HRHKEHKKDKDKDREKSKHSNSEHKDSEKKHKEKEKTKHK

SEQ ID NO: 87 - protein, 46 aa, Rattus norvegicus
HRHKEHKKDKDKDREKSKHSNSEHKDSEKKHKEKEKTKHKDGSSDK

SEQ ID NO: 88 - protein, 48 aa, Rattus norvegicus
HRHKEHKKDKDKDREKSKHSNSEHKDSEKKHKEKEKTKHKDGSSDKHK

SEQ ID NO: 89 - protein, 50 aa, Rattus norvegicus
HRHKEHKKDKDKDREKSKHSNSEHKDSEKKHKEKEKTKHKDGSSDKHKDK

SEQ ID NO: 90 - protein, 8 aa, Rattus norvegicus
HKEHKKDK

SEQ ID NO: 91 - protein, 10 aa, Rattus norvegicus
HKEHKKDKDK

SEQ ID NO: 92 - protein, 14 aa, Rattus norvegicus
HKEHKKDKDKDREK

SEQ ID NO: 93 - protein, 16 aa, Rattus norvegicus
HKEHKKDKDKDREKSK

SEQ ID NO: 94 - protein, 23 aa, Rattus norvegicus
HKEHKKDKDKDREKSKHSNSEHK

SEQ ID NO: 95 - protein, 30 aa, Rattus norvegicus
HKEHKKDKDKDREKSKHSNSEHKDSEKKHK

SEQ ID NO: 96 - protein, 32 aa, Rattus norvegicus
HKEHKKDKDKDREKSKHSNSEHKDSEKKHKEK

SEQ ID NO: 97 - protein, 34 aa, Rattus norvegicus
HKEHKKDKDKDREKSKHSNSEHKDSEKKHKEKEK

SEQ ID NO: 98 - protein, 36 aa, Rattus norvegicus
HKEHKKDKDKDREKSKHSNSEHKDSEKKHKEKEKTK

SEQ ID NO: 99 - protein, 38 aa, Rattus norvegicus
HKEHKKDKDKDREKSKHSNSEHKDSEKKHKEKEKTKHK

SEQ ID NO: 100 - protein, 44 aa, Rattus norvegicus
HKEHKKDKDKDREKSKHSNSEHKDSEKKHKEKEKTKHKDGSSDK

SEQ ID NO: 101 - protein, 46 aa, Rattus norvegicus
HKEHKKDKDKDREKSKHSNSEHKDSEKKHKEKEKTKHKDGSSDKHK

SEQ ID NO: 102 - protein, 48 aa, Rattus norvegicus
HKEHKKDKDKDREKSKHSNSEHKDSEKKHKEKEKTKHKDGSSDKHKDK

SEQ ID NO: 103 - protein, 50 aa, Rattus norvegicus
HKEHKKDKDKDREKSKHSNSEHKDSEKKHKEKEKTKHKDGSSDKHKDKHK

SEQ ID NO: 104 - protein, 7 aa, Rattus norvegicus
KEHKKDK

SEQ ID NO: 105 - protein, 9 aa, Rattus norvegicus
KEHKKDKDK

SEQ ID NO: 106 - protein, 16 aa, Rattus norvegicus
KEHKKDKDKDREKSKH

SEQ ID NO: 107 - protein, 21 aa, Rattus norvegicus
KEHKKDKDKDREKSKHSNSEH

SEQ ID NO: 108 - protein, 28 aa, Rattus norvegicus
KEHKKDKDKDREKSKHSNSEHKDSEKKH

SEQ ID NO: 109 - protein, 36 aa, Rattus norvegicus
KEHKKDKDKDREKSKHSNSEHKDSEKKHKEKEKTKH

SEQ ID NO: 110 - protein, 44 aa, Rattus norvegicus
KEHKKDKDKDREKSKHSNSEHKDSEKKHKEKEKTKHKDGSSDKH

SEQ ID NO: 111 - protein, 48 aa, Rattus norvegicus
KEHKKDKDKDREKSKHSNSEHKDSEKKHKEKEKTKHKDGSSDKHKDKH

SEQ ID NO: 112 - protein, 51 aa, Rattus norvegicus
HKKDKDKDREKSKHSNSEHKDSEKKHKEKEKTKHKDGSSDKHKDKHKDRDK

SEQ ID NO: 113 - protein, 11 aa, Rattus norvegicus
HKKDKDKDREK

SEQ ID NO: 114 - protein, 13 aa, Rattus norvegicus
HKKDKDKDREKSK

SEQ ID NO: 115 - protein, 20 aa, Rattus norvegicus
HKKDKDKDREKSKHSNSEHK

SEQ ID NO: 116 - protein, 27 aa, Rattus norvegicus
HKKDKDKDREKSKHSNSEHKDSEKKHK

SEQ ID NO: 117 - protein, 29 aa, Rattus norvegicus
HKKDKDKDREKSKHSNSEHKDSEKKHKEK

SEQ ID NO: 118 - protein, 31 aa, Rattus norvegicus
HKKDKDKDREKSKHSNSEHKDSEKKHKEKEK

SEQ ID NO: 119 - protein, 33 aa, Rattus norvegicus
HKKDKDKDREKSKHSNSEHKDSEKKHKEKEKTK

SEQ ID NO: 120 - protein, 35 aa, Rattus norvegicus
HKKDKDKDREKSKHSNSEHKDSEKKHKEKEKTKHK

SEQ ID NO: 121 - protein, 41 aa, Rattus norvegicus
HKKDKDKDREKSKHSNSEHKDSEKKHKEKEKTKHKDGSSDK

SEQ ID NO: 122 - protein, 43 aa, Rattus norvegicus
HKKDKDKDREKSKHSNSEHKDSEKKHKEKEKTKHKDGSSDKHK

SEQ ID NO: 123 - protein, 45 aa, Rattus norvegicus
HKKDKDKDREKSKHSNSEHKDSEKKHKEKEKTKHKDGSSDKHKDK

SEQ ID NO: 124 - protein, 47 aa, Rattus norvegicus
HKKDKDKDREKSKHSNSEHKDSEKKHKEKEKTKHKDGSSDKHKDKHK

SEQ ID NO: 125 - protein, 13 aa, Rattus norvegicus
KKDKDKDREKSKH

SEQ ID NO: 126 - protein, 18 aa, Rattus norvegicus
KKDKDKDREKSKHSNSEH

SEQ ID NO: 127 - protein, 25 aa, Rattus norvegicus
KKDKDKDREKSKHSNSEHKDSEKKH

SEQ ID NO: 128 - protein, 33 aa, Rattus norvegicus
KKDKDKDREKSKHSNSEHKDSEKKHKEKEKTKH

SEQ ID NO: 129 - protein, 41 aa, Rattus norvegicus
KKDKDKDREKSKHSNSEHKDSEKKHKEKEKTKHKDGSSDKH

SEQ ID NO: 130 - protein, 45 aa, Rattus norvegicus
KKDKDKDREKSKHSNSEHKDSEKKHKEKEKTKHKDGSSDKHKDKH

SEQ ID NO: 131 - protein, 12 aa, Rattus norvegicus
KDKDKDREKSKH

SEQ ID NO: 132 - protein, 17 aa, Rattus norvegicus
KDKDKDREKSKHSNSEH

SEQ ID NO: 133 - protein, 24 aa, Rattus norvegicus
KDKDKDREKSKHSNSEHKDSEKKH

SEQ ID NO: 134 - protein, 32 aa, Rattus norvegicus
KDKDKDREKSKHSNSEHKDSEKKHKEKEKTKH

SEQ ID NO: 135 - protein, 40 aa, Rattus norvegicus
KDKDKDREKSKHSNSEHKDSEKKHKEKEKTKHKDGSSDKH

SEQ ID NO: 136 - protein, 44 aa, Rattus norvegicus
KDKDKDREKSKHSNSEHKDSEKKHKEKEKTKHKDGSSDKHKDKH

SEQ ID NO: 137 - protein, 10 aa, Rattus norvegicus
KDKDREKSKH

SEQ ID NO: 138 - protein, 15 aa, Rattus norvegicus
KDKDREKSKHSNSEH

SEQ ID NO: 139 - protein, 22 aa, Rattus norvegicus
KDKDREKSKHSNSEHKDSEKKH

SEQ ID NO: 140 - protein, 30 aa, Rattus norvegicus
KDKDREKSKHSNSEHKDSEKKHKEKEKTKH

SEQ ID NO: 141 - protein, 38 aa, Rattus norvegicus
KDKDREKSKHSNSEHKDSEKKHKEKEKTKHKDGSSDKH

SEQ ID NO: 142 - protein, 42 aa, Rattus norvegicus
KDKDREKSKHSNSEHKDSEKKHKEKEKTKHKDGSSDKHKDKH

SEQ ID NO: 143 - protein, 8 aa, Rattus norvegicus
KDREKSKH

SEQ ID NO: 144 - protein, 13 aa, Rattus norvegicus
KDREKSKHSNSEH

SEQ ID NO: 145 - protein, 20 aa, Rattus norvegicus
KDREKSKHSNSEHKDSEKKH

SEQ ID NO: 146 - protein, 28 aa, Rattus norvegicus
KDREKSKHSNSEHKDSEKKHKEKEKTKH

SEQ ID NO: 147 - protein, 36 aa, Rattus norvegicus
KDREKSKHSNSEHKDSEKKHKEKEKTKHKDGSSDKH

SEQ ID NO: 148 - protein, 40 aa, Rattus norvegicus
KDREKSKHSNSEHKDSEKKHKEKEKTKHKDGSSDKHKDKH

SEQ ID NO: 149 - protein, 10 aa, Rattus norvegicus
KSKHSNSEHK

SEQ ID NO: 150 - protein, 16 aa, Rattus norvegicus
KSKHSNSEHKDSEKKH

SEQ ID NO: 151 - protein, 24 aa, Rattus norvegicus
KSKHSNSEHKDSEKKHKEKEKTKH

SEQ ID NO: 152 - protein, 32 aa, Rattus norvegicus
KSKHSNSEHKDSEKKHKEKEKTKHKDGSSDKH

SEQ ID NO: 153 - protein, 36 aa, Rattus norvegicus
KSKHSNSEHKDSEKKHKEKEKTKHKDGSSDKHKDKH

SEQ ID NO: 154 - protein, 8 aa, Rattus norvegicus
KHSNSEHK

SEQ ID NO: 155 - protein, 14 aa, Rattus norvegicus
KHSNSEHKDSEKKH

SEQ ID NO: 156 - protein, 22 aa, Rattus norvegicus
KHSNSEHKDSEKKHKEKEKTKH

SEQ ID NO: 157 - protein, 30 aa, Rattus norvegicus
KHSNSEHKDSEKKHKEKEKTKHKDGSSDKH

SEQ ID NO: 158 - protein, 34 aa, Rattus norvegicus
KHSNSEHKDSEKKHKEKEKTKHKDGSSDKHKDKH

SEQ ID NO: 159 - protein, 14 aa, Rattus norvegicus
HSNSEHKDSEKKHK

SEQ ID NO: 160 - protein, 16 aa, Rattus norvegicus
HSNSEHKDSEKKHKEK

SEQ ID NO: 161 - protein, 18 aa, Rattus norvegicus
HSNSEHKDSEKKHKEKEK

SEQ ID NO: 162 - protein, 20 aa, Rattus norvegicus
HSNSEHKDSEKKHKEKEKTK

SEQ ID NO: 163 - protein, 22 aa, Rattus norvegicus
HSNSEHKDSEKKHKEKEKTKHK

SEQ ID NO: 164 - protein, 28 aa, Rattus norvegicus
HSNSEHKDSEKKHKEKEKTKHKDGSSDK

SEQ ID NO: 165 - protein, 30 aa, Rattus norvegicus
HSNSEHKDSEKKHKEKEKTKHKDGSSDKHK

SEQ ID NO: 166 - protein, 32 aa, Rattus norvegicus
HSNSEHKDSEKKHKEKEKTKHKDGSSDKHKDK

SEQ ID NO: 167 - protein, 34 aa, Rattus norvegicus
HSNSEHKDSEKKHKEKEKTKHKDGSSDKHKDKHK

SEQ ID NO: 168 - protein, 38 aa, Rattus norvegicus
HSNSEHKDSEKKHKEKEKTKHKDGSSDKHKDKHKDRDK

SEQ ID NO: 169 - protein, 40 aa, Rattus norvegicus
HSNSEHKDSEKKHKEKEKTKHKDGSSDKHKDKHKDRDKEK

SEQ ID NO: 170 - protein, 42 aa, Rattus norvegicus
HSNSEHKDSEKKHKEKEKTKHKDGSSDKHKDKHKDRDKEKRK

SEQ ID NO: 171 - protein, 45 aa, Rattus norvegicus
HSNSEHKDSEKKHKEKEKTKHKDGSSDKHKDKHKDRDKEKRKEEK

SEQ ID NO: 172 - protein, 9 aa, Rattus norvegicus
HKDSEKKHK

SEQ ID NO: 173 - protein, 11 aa, Rattus norvegicus
HKDSEKKHKEK

SEQ ID NO: 174 - protein, 13 aa, Rattus norvegicus
HKDSEKKHKEKEK

SEQ ID NO: 175 - protein, 15 aa, Rattus norvegicus
HKDSEKKHKEKEKTK

SEQ ID NO: 176 - protein, 17 aa, Rattus norvegicus
HKDSEKKHKEKEKTKHK

SEQ ID NO: 177 - protein, 23 aa, Rattus norvegicus
HKDSEKKHKEKEKTKHKDGSSDK

SEQ ID NO: 178 - protein, 25 aa, Rattus norvegicus
HKDSEKKHKEKEKTKHKDGSSDKHK

SEQ ID NO: 179 - protein, 27 aa, Rattus norvegicus
HKDSEKKHKEKEKTKHKDGSSDKHKDK

SEQ ID NO: 180 - protein, 29 aa, Rattus norvegicus
HKDSEKKHKEKEKTKHKDGSSDKHKDKHK

SEQ ID NO: 181 - protein, 33 aa, Rattus norvegicus
HKDSEKKHKEKEKTKHKDGSSDKHKDKHKDRDK

SEQ ID NO: 182 - protein, 35 aa, Rattus norvegicus
HKDSEKKHKEKEKTKHKDGSSDKHKDKHKDRDKEK

SEQ ID NO: 183 - protein, 37 aa, Rattus norvegicus
HKDSEKKHKEKEKTKHKDGSSDKHKDKHKDRDKEKRK

SEQ ID NO: 184 - protein, 40 aa, Rattus norvegicus
HKDSEKKHKEKEKTKHKDGSSDKHKDKHKDRDKEKRKEEK

SEQ ID NO: 185 - protein, 48 aa, Rattus norvegicus
HKDSEKKHKEKEKTKHKDGSSDKHKDKHKDRDKEKRKEEKIRAAGDAK

SEQ ID NO: 186 - protein, 50 aa, Rattus norvegicus
HKDSEKKHKEKEKTKHKDGSSDKHKDKHKDRDKEKRKEEKIRAAGDAKIK

SEQ ID NO: 187 - protein, 8 aa, Rattus norvegicus
KDSEKKHK

SEQ ID NO: 188 - protein, 10 aa, Rattus norvegicus
KDSEKKHKEK

SEQ ID NO: 189 - protein, 15 aa, Rattus norvegicus
KDSEKKHKEKEKTKH

SEQ ID NO: 190 - protein, 23 aa, Rattus norvegicus
KDSEKKHKEKEKTKHKDGSSDKH

SEQ ID NO: 191 - protein, 27 aa, Rattus norvegicus
KDSEKKHKEKEKTKHKDGSSDKHKDKH

SEQ ID NO: 192 - protein, 8 aa, Rattus norvegicus
KKHKEKEK

SEQ ID NO: 193 - protein, 11 aa, Rattus norvegicus
KKHKEKEKTKH

SEQ ID NO: 194 - protein, 10 aa, Rattus norvegicus
KKHKEKEKTK

SEQ ID NO: 195 - protein, 19 aa, Rattus norvegicus
KKHKEKEKTKHKDGSSDKH

SEQ ID NO: 196 - protein, 23 aa, Rattus norvegicus
KKHKEKEKTKHKDGSSDKHKDKH

SEQ ID NO: 197 - protein, 7 aa, Rattus norvegicus
KHKEKEK

SEQ ID NO: 198 - protein, 10 aa, Rattus norvegicus
KHKEKEKTKH

SEQ ID NO: 199 - protein, 9 aa, Rattus norvegicus
KHKEKEKTK

SEQ ID NO: 200 - protein, 11 aa, Rattus norvegicus
KHKEKEKTKHK

SEQ ID NO: 201 - protein, 18 aa, Rattus norvegicus
KHKEKEKTKHKDGSSDKH

SEQ ID NO: 202 - protein, 22 aa, Rattus norvegicus
KHKEKEKTKHKDGSSDKHKDKH

SEQ ID NO: 203 - protein, 8 aa, Rattus norvegicus
HKEKEKTK

SEQ ID NO: 204 - protein, 10 aa, Rattus norvegicus
HKEKEKTKHK

SEQ ID NO: 205 - protein, 41 aa, Rattus norvegicus
HKEKEKTKHKDGSSDKHKDKHKDRDKEKRKEEKIRAAGDAK

SEQ ID NO: 206 - protein, 43 aa, Rattus norvegicus
HKEKEKTKHKDGSSDKHKDKHKDRDKEKRKEEKIRAAGDAKIK

SEQ ID NO: 207 - protein, 16 aa, Rattus norvegicus
HKEKEKTKHKDGSSDK

SEQ ID NO: 208 - protein, 18 aa, Rattus norvegicus
HKEKEKTKHKDGSSDKHK

SEQ ID NO: 209 - protein, 20 aa, Rattus norvegicus
HKEKEKTKHKDGSSDKHKDK

SEQ ID NO: 210 - protein, 22 aa, Rattus norvegicus
HKEKEKTKHKDGSSDKHKDKHK

SEQ ID NO: 211 - protein, 26 aa, Rattus norvegicus
HKEKEKTKHKDGSSDKHKDKHKDRDK

SEQ ID NO: 212 - protein, 28 aa, Rattus norvegicus
HKEKEKTKHKDGSSDKHKDKHKDRDKEK

SEQ ID NO: 213 - protein, 30 aa, Rattus norvegicus
HKEKEKTKHKDGSSDKHKDKHKDRDKEKRK

SEQ ID NO: 214 - protein, 33 aa, Rattus norvegicus
HKEKEKTKHKDGSSDKHKDKHKDRDKEKRKEEK

SEQ ID NO: 215 - protein, 8 aa, Rattus norvegicus
KEKEKTKH

SEQ ID NO: 216 - protein, 9 aa, Rattus norvegicus
KEKEKTKHK

SEQ ID NO: 217 - protein, 16 aa, Rattus norvegicus
KEKEKTKHKDGSSDKH

SEQ ID NO: 218 - protein, 20 aa, Rattus norvegicus
KEKEKTKHKDGSSDKHKDKH

SEQ ID NO: 219 - protein, 7 aa, Rattus norvegicus
KEKTKHK

SEQ ID NO: 220 - protein, 14 aa, Rattus norvegicus
KEKTKHKDGSSDKH

SEQ ID NO: 221 - protein, 18 aa, Rattus norvegicus
KEKTKHKDGSSDKHKDKH

SEQ ID NO: 222 - protein, 11 aa, Rattus norvegicus
KTKHKDGSSDK

SEQ ID NO: 223 - protein, 12 aa, Rattus norvegicus
KTKHKDGSSDKH

SEQ ID NO: 224 - protein, 16 aa, Rattus norvegicus
KTKHKDGSSDKHKDKH

SEQ ID NO: 225 - protein, 9 aa, Rattus norvegicus
KHKDGSSDK

SEQ ID NO: 226 - protein, 10 aa, Rattus norvegicus
KHKDGSSDKH

SEQ ID NO: 227 - protein, 11 aa, Rattus norvegicus
KHKDGSSDKHK

SEQ ID NO: 228 - protein, 14 aa, Rattus norvegicus
KHKDGSSDKHKDKH

SEQ ID NO: 229 - protein, 33 aa, Rattus norvegicus
HKDGSSDKHKDKHKDRDKEKRKEEKIRAAGDAK

SEQ ID NO: 230 - protein, 35 aa, Rattus norvegicus
HKDGSSDKHKDKHKDRDKEKRKEEKIRAAGDAKIK

SEQ ID NO: 231 - protein, 8 aa, Rattus norvegicus
HKDGSSDK

SEQ ID NO: 232 - protein, 10 aa, Rattus norvegicus
HKDGSSDKHK

SEQ ID NO: 233 - protein, 12 aa, Rattus norvegicus
HKDGSSDKHKDK

SEQ ID NO: 234 - protein, 14 aa, Rattus norvegicus
HKDGSSDKHKDKHK

SEQ ID NO: 235 - protein, 18 aa, Rattus norvegicus
HKDGSSDKHKDKHKDRDK

SEQ ID NO: 236 - protein, 20 aa, Rattus norvegicus
HKDGSSDKHKDKHKDRDKEK

SEQ ID NO: 237 - protein, 22 aa, Rattus norvegicus
HKDGSSDKHKDKHKDRDKEKRK

SEQ ID NO: 238 - protein, 25 aa, Rattus norvegicus
HKDGSSDKHKDKHKDRDKEKRKEEK

SEQ ID NO: 239 - protein, 8 aa, Rattus norvegicus
KDGSSDKH

SEQ ID NO: 240 - protein, 9 aa, Rattus norvegicus
KDGSSDKHK

SEQ ID NO: 241 - protein, 11 aa, Rattus norvegicus
KDGSSDKHKDK

SEQ ID NO: 242 - protein, 12 aa, Rattus norvegicus
KDGSSDKHKDKH

SEQ ID NO: 243 - protein, 7 aa, Rattus norvegicus
KHKDKHK

SEQ ID NO: 244 - protein, 11 aa, Rattus norvegicus
KHKDKHKDRDK

SEQ ID NO: 245 - protein, 25 aa, Rattus norvegicus
HKDKHKDRDKEKRKEEKIRAAGDAK

SEQ ID NO: 246 - protein, 27 aa, Rattus norvegicus
HKDKHKDRDKEKRKEEKIRAAGDAKIK

SEQ ID NO: 247 - protein, 10 aa, Rattus norvegicus
HKDKHKDRDK

SEQ ID NO: 248 - protein, 12 aa, Rattus norvegicus
HKDKHKDRDKEK

SEQ ID NO: 249 - protein, 14 aa, Rattus norvegicus
HKDKHKDRDKEKRK

SEQ ID NO: 250 - protein, 17 aa, Rattus norvegicus
HKDKHKDRDKEKRKEEK

SEQ ID NO: 251 - protein, 9 aa, Rattus norvegicus
KDKHKDRDK

SEQ ID NO: 252 - protein, 11 aa, Rattus norvegicus
KDKHKDRDKEK

SEQ ID NO: 253 - protein, 7 aa, Rattus norvegicus
KHKDRDK

SEQ ID NO: 254 - protein, 9 aa, Rattus norvegicus
KHKDRDKEK

SEQ ID NO: 255 - protein, 11 aa, Rattus norvegicus
KHKDRDKEKRK

SEQ ID NO: 256 - protein, 8 aa, Rattus norvegicus
HKDRDKEK

SEQ ID NO: 257 - protein, 10 aa, Rattus norvegicus
HKDRDKEKRK

SEQ ID NO: 258 - protein, 21 aa, Rattus norvegicus
HKDRDKEKRKEEKIRAAGDAK

SEQ ID NO: 259 - protein, 23 aa, Rattus norvegicus
HKDRDKEKRKEEKIRAAGDAKIK

SEQ ID NO: 260 - protein, 13 aa, Rattus norvegicus
HKDRDKEKRKEEK

SEQ ID NO: 261 - protein, 51 aa, Rattus norvegicus
KLKKPKNKDKDKKVAEPDNKKKKAKKEEEQKWKWWEEERYPEGIKWKFLEH

SEQ ID NO: 262 - protein, 49 aa, Rattus norvegicus
KKPKNKDKDKKVAEPDNKKKKAKKEEEQKWKWWEEERYPEGIKWKFLEH

SEQ ID NO: 263 - protein, 48 aa, Rattus norvegicus
KPKNKDKDKKVAEPDNKKKKAKKEEEQKWKWWEEERYPEGIKWKFLEH

SEQ ID NO: 264 - protein, 46 aa, Rattus norvegicus
KNKDKDKKVAEPDNKKKKAKKEEEQKWKWWEEERYPEGIKWKFLEH

SEQ ID NO: 265 - protein, 42 aa, Rattus norvegicus
KDKKVAEPDNKKKKAKKEEEQKWKWWEEERYPEGIKWKFLEH

SEQ ID NO: 266 - protein, 40 aa, Rattus norvegicus
KKVAEPDNKKKKAKKEEEQKWKWWEEERYPEGIKWKFLEH

SEQ ID NO: 267 - protein, 39 aa, Rattus norvegicus
KVAEPDNKKKKAKKEEEQKWKWWEEERYPEGIKWKFLEH

SEQ ID NO: 268 - protein, 32 aa, Rattus norvegicus
KKKKAKKEEEQKWKWWEEERYPEGIKWKFLEH

SEQ ID NO: 269 - protein, 31 aa, Rattus norvegicus
KKKAKKEEEQKWKWWEEERYPEGIKWKFLEH

SEQ ID NO: 270 - protein, 30 aa, Rattus norvegicus
KKAKKEEEQKWKWWEEERYPEGIKWKFLEH

SEQ ID NO: 271 - protein, 29 aa, Rattus norvegicus
KAKKEEEQKWKWWEEERYPEGIKWKFLEH

SEQ ID NO: 272 - protein, 27 aa, Rattus norvegicus
KKEEEQKWKWWEEERYPEGIKWKFLEH

SEQ ID NO: 273 - protein, 26 aa, Rattus norvegicus
KEEEQKWKWWEEERYPEGIKWKFLEH

SEQ ID NO: 274 - protein, 51 aa, Rattus norvegicus
KWKFLEHKGPVFAPPYEPLPEGVKFYYDGKVMKLSPKAEEVATFFAKMLDH

SEQ ID NO: 275 - protein, 8 aa, Rattus norvegicus
KWKFLEHK

SEQ ID NO: 276 - protein, 24 aa, Rattus norvegicus
HKGPVFAPPYEPLPEGVKFYYDGK

SEQ ID NO: 277 - protein, 27 aa, Rattus norvegicus
HKGPVFAPPYEPLPEGVKFYYDGKVMK

SEQ ID NO: 278 - protein, 31 aa, Rattus norvegicus
HKGPVFAPPYEPLPEGVKFYYDGKVMKLSPK

SEQ ID NO: 279 - protein, 41 aa, Rattus norvegicus
HKGPVFAPPYEPLPEGVKFYYDGKVMKLSPKAEEVATFFAK

SEQ ID NO: 280 - protein, 50 aa, Rattus norvegicus
HKGPVFAPPYEPLPEGVKFYYDGKVMKLSPKAEEVATFFAKMLDHEYTTK

SEQ ID NO: 281 - protein, 28 aa, Rattus norvegicus
KFYYDGKVMKLSPKAEEVATFFAKMLDH

SEQ ID NO: 282 - protein, 22 aa, Rattus norvegicus
KVMKLSPKAEEVATFFAKMLDH

SEQ ID NO: 283 - protein, 15 aa, Rattus norvegicus
KAEEVATFFAKMLDH

SEQ ID NO: 284 - protein, 10 aa, Rattus norvegicus
KMLDHEYTTK

SEQ ID NO: 285 - protein, 15 aa, Rattus norvegicus
HEYTTKEIFRKNFFK

SEQ ID NO: 286 - protein, 19 aa, Rattus norvegicus
HEYTTKEIFRKNFFKDWRK

SEQ ID NO: 287 - protein, 34 aa, Rattus norvegicus
HEYTTKEIFRKNFFKDWRKEMTNDEKNTITNLSK

SEQ ID NO: 288 - protein, 26 aa, Rattus norvegicus
HEYTTKEIFRKNFFKDWRKEMTNDEK

SEQ ID NO: 289 - protein, 37 aa, Rattus norvegicus
KAQSEARKQMSKEEKLKIKEENEKLLKEYGFCVMDNH

SEQ ID NO: 290 - protein, 30 aa, Rattus norvegicus
KQMSKEEKLKIKEENEKLLKEYGFCVMDNH

SEQ ID NO: 291 - protein, 51 aa, Rattus norvegicus
KQMSKEEKLKIKEENEKLLKEYGFCVMDNHRERIANFKIEPPGLFRGRGNH

SEQ ID NO: 292 - protein, 26 aa, Rattus norvegicus
KEEKLKIKEENEKLLKEYGFCVMDNH

SEQ ID NO: 293 - protein, 47 aa, Rattus norvegicus
KEEKLKIKEENEKLLKEYGFCVMDNHRERIANFKIEPPGLFRGRGNH

SEQ ID NO: 294 - protein, 23 aa, Rattus norvegicus
KLKIKEENEKLLKEYGFCVMDNH

SEQ ID NO: 295 - protein, 44 aa, Rattus norvegicus
KLKIKEENEKLLKEYGFCVMDNHRERIANFKIEPPGLFRGRGNH

SEQ ID NO: 296 - protein, 21 aa, Rattus norvegicus
KIKEENEKLLKEYGFCVMDNH

SEQ ID NO: 297 - protein, 42 aa, Rattus norvegicus
KIKEENEKLLKEYGFCVMDNHRERIANFKIEPPGLFRGRGNH

SEQ ID NO: 298 - protein, 19 aa, Rattus norvegicus
KEENEKLLKEYGFCVMDNH

SEQ ID NO: 299 - protein, 40 aa, Rattus norvegicus
KEENEKLLKEYGFCVMDNHRERIANFKIEPPGLFRGRGNH

SEQ ID NO: 300 - protein, 34 aa, Rattus norvegicus
HPKMGMLKRRIMPEDIIINCSKDAKVPSPPPGHK

SEQ ID NO: 301 - protein, 43 aa, Rattus norvegicus
HPKMGMLKRRIMPEDIIINCSKDAKVPSPPPGHKWKEVRHDNK

SEQ ID NO: 302 - protein, 10 aa, Rattus norvegicus
KVPSPPPGHK

SEQ ID NO: 303 - protein, 16 aa, Rattus norvegicus
KVPSPPPGHKWKEVRH

SEQ ID NO: 304 - protein, 11 aa, Rattus norvegicus
HKWKEVRHDNK

SEQ ID NO: 305 - protein, 45 aa, Rattus norvegicus
HKWKEVRHDNKVTWLVSWTENIQGSIKYIMLNPSSRIKGEKDWQK

SEQ ID NO: 306 - protein, 10 aa, Rattus norvegicus
KWKEVRHDNK

SEQ ID NO: 307 - protein, 8 aa, Rattus norvegicus
KEVRHDNK

SEQ ID NO: 308 - protein, 38 aa, Rattus norvegicus
HDNKVTWLVSWTENIQGSIKYIMLNPSSRIKGEKDWQK

SEQ ID NO: 309 - protein, 46 aa, Rattus norvegicus
HDNKVTWLVSWTENIQGSIKYIMLNPSSRIKGEKDWQKYETARRLK

SEQ ID NO: 310 - protein, 47 aa, Rattus norvegicus
HDNKVTWLVSWTENIQGSIKYIMLNPSSRIKGEKDWQKYETARRLKK

SEQ ID NO: 311 - protein, 28 aa, Rattus norvegicus
KLALRAGNEKEEGETADTVGCCSLRVEH

SEQ ID NO: 312 - protein, 32 aa, Rattus norvegicus
KLALRAGNEKEEGETADTVGCCSLRVEHINLH

SEQ ID NO: 313 - protein, 30 aa, Rattus norvegicus
HINLHPELDGQEYVVEFDFPGKDSIRYYNK

SEQ ID NO: 314 - protein, 39 aa, Rattus norvegicus
HINLHPELDGQEYVVEFDFPGKDSIRYYNKVPVEKRVFK

SEQ ID NO: 315 - protein, 48 aa, Rattus norvegicus
HINLHPELDGQEYVVEFDFPGKDSIRYYNKVPVEKRVFKNLQLFMENK

SEQ ID NO: 316 - protein, 26 aa, Rattus norvegicus
HPELDGQEYVVEFDFPGKDSIRYYNK

SEQ ID NO: 317 - protein, 35 aa, Rattus norvegicus
HPELDGQEYVVEFDFPGKDSIRYYNKVPVEKRVFK

SEQ ID NO: 318 - protein, 44 aa, Rattus norvegicus
HPELDGQEYVVEFDFPGKDSIRYYNKVPVEKRVFKNLQLFMENK

SEQ ID NO: 319 - protein, 45 aa, Rattus norvegicus
KDSIRYYNKVPVEKRVFKNLQLFMENKQPEDDLFDRLNTGILNKH

SEQ ID NO: 320 - protein, 37 aa, Rattus norvegicus
KVPVEKRVFKNLQLFMENKQPEDDLFDRLNTGILNKH

SEQ ID NO: 321 - protein, 28 aa, Rattus norvegicus
KNLQLFMENKQPEDDLFDRLNTGILNKH

SEQ ID NO: 322 - protein, 19 aa, Rattus norvegicus
HQRAPPKTFEKSMMNLQSK

SEQ ID NO: 323 - protein, 32 aa, Rattus norvegicus
HQRAPPKTFEKSMMNLQSKIDAKKDQLADARK

SEQ ID NO: 324 - protein, 38 aa, Rattus norvegicus
HQRAPPKTFEKSMMNLQSKIDAKKDQLADARKDLKSAK

SEQ ID NO: 325 - protein, 42 aa, Rattus norvegicus
HQRAPPKTFEKSMMNLQSKIDAKKDQLADARKDLKSAKADAK

SEQ ID NO: 326 - protein, 45 aa, Rattus norvegicus
HQRAPPKTFEKSMMNLQSKIDAKKDQLADARKDLKSAKADAKVMK

SEQ ID NO: 327 - protein, 48 aa, Rattus norvegicus
HQRAPPKTFEKSMMNLQSKIDAKKDQLADARKDLKSAKADAKVMKDAK

SEQ ID NO: 328 - protein, 50 aa, Rattus norvegicus
HQRAPPKTFEKSMMNLQSKIDAKKDQLADARKDLKSAKADAKVMKDAKTK

SEQ ID NO: 329 - protein, 51 aa, Rattus norvegicus
HQRAPPKTFEKSMMNLQSKIDAKKDQLADARKDLKSAKADAKVMKDAKTKK

SEQ ID NO: 330 - protein, 10 aa, Homo sapiens
KAGVAFLHKK